(19) United States
(12) Patent Application Publication
(10) Pub. No.: US 2025/0066408 A1
(43) Pub. Date: Feb. 27, 2025

(54) Polyol-Derived Compounds

(71) Applicant: Arxada AG, Visp (CH)

(72) Inventors: Ulrich Mayerhoeffer, Visp (CH); Sebastian Benz, Visp (CH); Janis Michel, Visp (CH); Florian Dardano, Visp (CH); Marianne Haedener, Visp (CN)

(21) Appl. No.: 18/948,612

(22) Filed: Nov. 15, 2024

Related U.S. Application Data
(63) Continuation of application No. 18/709,672, filed on May 13, 2024.

(30) Foreign Application Priority Data
Nov. 12, 2021 (EP) 21208071.7

(51) Int. Cl. C07H 13/04; C07C 69/67

(52) U.S. Cl. CPC C07H 13/04; C07C 69/67; C07B 2200/07

(57) ABSTRACT

The present invention relates to polyol-derived compounds and processes preparing the same.

### TECHNICAL FIELD

The present invention relates to polyol-derived compounds and processes preparing the same.

### TECHNOLOGICAL BACKGROUND

Acetoacetylated polyalcohols and p-hydroxy butyric acid (BHB) esters of polyalcohols prepared therefrom are valuable compounds with a versatile utilization for example as parenteral nutrients or for the treatment of certain diseases.

US 2019/117612 A1 pertains to the field of migraine headaches and the management of the symptomology thereof using 3-hydroxybutyrate glycerides.

US 2018/193300 A1 pertains to a method of treatment of mild to moderate non-penetrating closed traumatic brain injury and mild to moderate traumatic brain injury due to surgical intervention using 3-hydroxybutyate glycerides.

Acetoacetylated polyalcohols and p-hydroxy butyric acid (BHB) esters of polyalcohols are usually prepared by coupling a polyalcohol such as glycerol with protected beta hydroxy butyric acid or acetoacetate esters. Both methods suffer from poor atom economy and result in more waste.

Moreover, BHB esters of polyalcohols usually have a low BHB content per polyalcohol unit. However, in order to increase BHB delivery efficiency, a high BHB content per polyalcohol unit would be desirable. Furthermore, protecting the BHB units in BHB esters of polyalcohols would enable the delivery of further BHB precursors, which upon hydrolysis are oxidized by the body to BHB, which further increasing BHB delivery efficiency.

Hence, there is a need for providing polyalcohols with a high BHB unit or acetoacetate concentration per polyalcohol unit. There is further a need for providing BHB esters of polyalcohols in which the BHB units are further functionalized or protected.

There is further a need for optimized processes for the synthesis of such acetoacetylated polyalcohols and β-hydroxy butyric acid (BHB) esters of polyalcohols having a high content of BHB units or acetoacetates per polyalcohol unit. There is further a need for optimized processes for the synthesis of BHB esters of polyalcohols in which the BHB units are further functionalized or protected.

### SUMMARY OF THE INVENTION

The inventors surprisingly found that the processes according to the present invention by reacting a diketene with a polyol or a β-hydroxyl butyric acid ester of a polyol provides an excellent method for producing stable and neutral analogues of beta hydroxy butyric acid. The reaction of a polyol or a p-hydroxyl butyric acid ester of a polyol with diketene and subsequent hydrogenation and optional esterification allows for facile access to the desired products. Using asymmetric hydrogenation provides access to enantiopure derivatives. Moreover, the processes according to the present invention allow for the synthesis of polyalcohols with a high BHB unit or acetoacetate concentration per polyalcohol unit.

Accordingly, the present invention provides a compound of formula 1

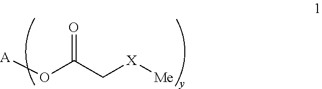

wherein
A is derived from an organic polyol with at least 4 hydroxyl groups,
X is —C(H)(OH)— or —C(H)(OR¹)—,
R¹ is selected from linear or branched C₁₋₁₂ alkyl, C₃₋₈ cycloalkyl, linear or branched C₂₋₁₂ hydroxyalkyl, linear or branched C₁₋₁₂ carboxyalkly, linear or branched and saturated or unsaturated C₁₋₂₄ alkanoyl, phenyl, and carboxyphenyl, and
y is from 1 to the number of hydroxyl groups of the initial organic polyol A.

In another aspect, the present invention provides a compound of formula 9

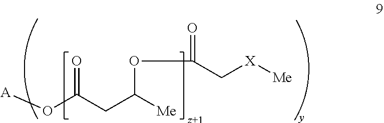

wherein
z is 0 or 1,
A is derived from an organic polyol with at least 3 hydroxyl groups,
X is —C(O)—, —C(H)(OH)—, or —C(H)(OR¹)—,
R¹ is selected from linear or branched C₁₋₁₂ alkyl, C₃₋₈ cycloalkyl, linear or branched C₂₋₁₂ hydroxyalkyl, linear or branched C₁₋₁₂ carboxyalkly, linear or branched and saturated or unsaturated C₁₋₂₄ alkanoyl, phenyl, and carboxyphenyl, and
y is from 1 to the number of hydroxyl groups of the initial polyol A.

In another aspect, the present invention provides a compound of formula 9

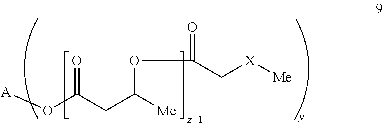

wherein
z is 0 or more,
A is derived from an organic polyol with at least 3 hydroxyl groups, provided that the organic polyol is not erythritol,
X is —C(O)—, —C(H)(OH)—, or —C(H)(OR¹)—,
R¹ is selected from linear or branched C₁₋₁₂ alkyl, C₃₋₈ cycloalkyl, linear or branched C₂₋₁₂ hydroxyalkyl, linear or branched C₁₋₁₂ carboxyalkly, linear or branched and saturated or unsaturated C₁₋₂₄ alkanoyl, phenyl, and carboxyphenyl, and
y is from 1 to the number of hydroxyl groups of the initial polyol A.

In another aspect, the present invention provides a process for the preparation of a compound of formula 1

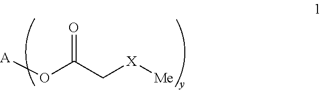

wherein
A is derived from an organic polyol with at least 4 hydroxyl groups,
X is —C(H)(OH)— or —C(H)(OR¹)—,
R¹ is selected from linear or branched C₁₋₁₂ alkyl, C₃₋₈ cycloalkyl, linear or branched C₂₋₁₂ hydroxyalkyl, linear or branched C₁₋₁₂ carboxyalkly, linear or branched and saturated or unsaturated C₁₋₂₄ alkanoyl, phenyl, and carboxyphenyl, and
y is from 1 to the number of hydroxyl groups of the initial organic polyol A;
wherein the process comprises:
   (i) reacting an organic polyol of formula 2 with diketene 3 resulting in the formation of a compound according to formula 4;

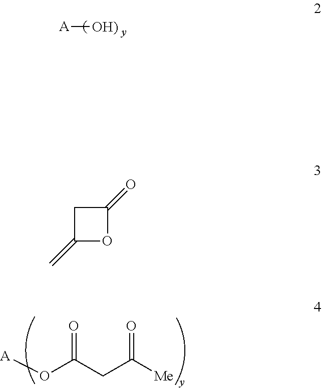

and
(iia) reacting the compound of formula 4 with hydrogen in the presence of a catalyst resulting in the formation of a compound according to formula 5,

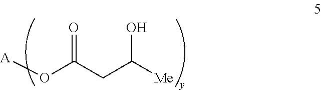

and optionally
(iib) reacting the compound 5 with a compound LG-R¹, wherein LG is a leaving group, resulting in the formation of a compound according to formula 6;

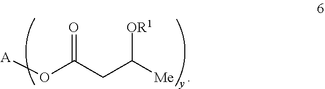

In another aspect, the present invention provides a process for the preparation of a compound of formula 9

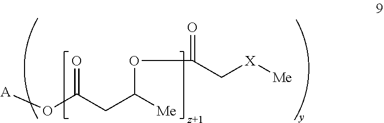

wherein
z is 0 or more,
A is derived from an organic polyol with at least 3 hydroxyl groups,
X is —C(O)—, —C(H)(OH)—, or —C(H)(OR¹)—,
R¹ is selected from linear or branched C₁₋₁₂ alkyl, C₃₋₈ cycloalkyl, linear or branched C₂₋₁₂ hydroxyalkyl, linear or branched C₁₋₁₂ carboxyalkly, linear or branched and saturated or unsaturated C₁₋₂₄ alkanoyl, phenyl, and carboxyphenyl, and
y is from 1 to the number of hydroxyl groups of the initial organic polyol A;
wherein the process comprises:
   (i) reacting a compound of formula 10 with diketene 3 resulting in the formation of a compound according to formula 11;

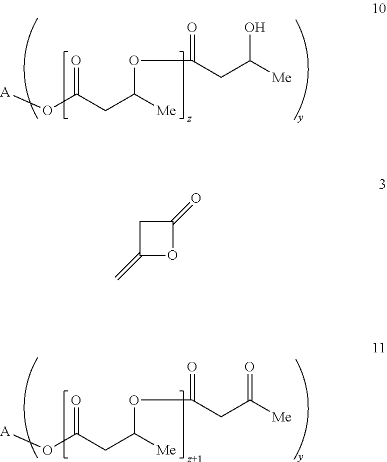

optionally
(iia) reacting the compound of formula 11 with hydrogen in the presence of a catalyst resulting in the formation of a compound according to formula 12,

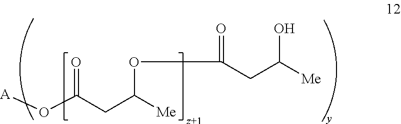

and optionally
(iib) reacting the compound of formula 12 with a compound LG-R¹, wherein LG is a leaving group, resulting in the formation of a compound according to formula 13;

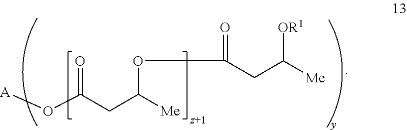

### DETAILED DESCRIPTION OF THE INVENTION

In the following, the invention will be explained in more detail.

### Definitions

In order for the present invention to be readily understood, several definitions of terms used in the course of the invention are set forth below.

According to the present invention, the term “linear or branched C₁₋₁₂ alkyl” refers to a straight-chained or branched saturated hydrocarbon group having 1 to 12 carbon atoms, such as 1, 2, 3, 4, 5, 6, 7, 8, 9, 10, 11 or 12 carbon atoms, including methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, 1,1-dimethylpropyl, 1,2-dimethylpropyl, hexyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethyl propyl, 1,2,2-trimethylpropyl, 1-ethyl-1-methylpropyl and 1-ethyl-2-methylpropyl.

According to the present invention, the term “C₃₋₈ cycloalkyl” refers to a monocyclic saturated hydrocarbon group having 3 to 8 carbon ring members, such as 2, 3, 4, 5, 6, 7, or 8 carbon ring members, including cyclopropyl, cyclobutyl, cyclopentyl, cyclohexyl, cycloheptyl, and cyclooctyl.

According to the present invention, the term “linear or branched C₂₋₁₂ hydroxyalkyl” refers to a straight-chained or branched saturated hydrocarbon group having 2 to 12 carbon atoms, such as 2, 3, 4, 5, 6, 7, 8, 9, 10, 11 or 12 carbon atoms, wherein at least one hydrogen atom is replaced by a hydroxy group, including 1-hydroxyethyl, 2-hydroxyethyl, 1-hydroxypropyl, 2-hydroxypropyl, 3-hydroxypropyl, 2-hydroxyisopropy, 1-hydroxybutyl, 2-hydroxybutyl, 3-hydroxybutyl, 4-hydroxybutyl, 1-hydroxypentyl, 2-hydroxypentyl, 3-hydroxypentyl, 4-hydroxypentyl, 5-hydroxypentyl, 1-hydroxyhexyl, 2-hydroxyhexyl, 3-hydroxyhexyl, 4-hydroxyhexyl, 5-hydroxyhexyl, and 6-hydroxyhexyl.

According to the present invention, the term “linear or branched C₁₋₁₂ carboxyalkyl” refers to a straight-chained or branched saturated hydrocarbon group having 1 to 12 carbon atoms as defined above, wherein at least one hydrogen atom is replaced by a carboxy group, including carboxymethyl, 1-carboxyethyl, 2-carboxyethyl, 1-methyl-2-carboxyethyl, 1-carboxypropyl, 2-carboxypropyl, 3-carboxypropyl, 1-methyl-2-carboxypropyl, 1-methyl-3-carboxypropyl, 1,1-dimethyl-2-carboxypropyl, 1,1-dimethyl-3-carboxypropyl, 1,2-dimethyl-3-carboxypropyl, 2,2-dimethyl-3-carboxypropyl, 1-carboxybutyl, 2-carboxybutyl, 3-carboxybutyl, 4-carboxybutyl, 1-methyl-4-carboxybutyl, 2-methyl-4-carboxybutyl, 3-methyl-4-carboxybutyl, 1,1-dimethyl-4-carboxybutyl, 1,2-dimethyl-4-carboxybutyl, 1,3-dimethyl-4-carboxybutyl, 2,2-dimethyl-4-carboxybutyl, 2,3-dimethyl-4-carboxybutyl, 3,3-dimethyl-4-carboxybutyl, 5-carboxypentyl, and 6-carboxyhexyl.

According to the present invention, the term “carboxyphenyl” refers to a phenol group, wherein at least one hydrogen atom is replaced by a carboxy group, such as, for example, o/m/p-carboxyphenol, including esters of the one or more carboxy functions, such as carboxymethyl, 1-carboxyethyl, 2-carboxyethyl, 1-methyl-2-carboxyethyl, 1-carboxypropyl, 2-carboxypropyl, 3-carboxypropyl, 1-methyl-2-carboxypropyl, 1-methyl-3-carboxypropyl, 1,1-dimethyl-2-carboxypropyl, 1,1-dimethyl-3-carboxypropyl, 1,2-dimethyl-3-carboxypropyl, 2,2-dimethyl-3-carboxypropyl, 1-carboxybutyl, 2-carboxybutyl, 3-carboxybutyl, 4-carboxybutyl, 1-methyl-4-carboxybutyl, 2-methyl-4-carboxybutyl, 3-methyl-4-carboxybutyl, 1,1-dimethyl-4-carboxybutyl, 1,2-dimethyl-4-carboxybutyl, 1,3-dimethyl-4-carboxybutyl, 2,2-dimethyl-4-carboxybutyl, 2,3-dimethyl-4-carboxybutyl, 3,3-dimethyl-4-carboxybutyl, 5-carboxypentyl, and 6-carboxyhexyl.

According to the present invention, the term “linear or branched and saturated or unsaturated C₁₋₂₄ alkanoyl” refers to a group —C(O)—R— wherein R is a linear or branched and saturated or unsaturated C₁₋₂₄ alkyl residue. Included are alkanoyls derived from fatty acids, e.g. medium-chain fatty acids such as caproic acid, caprylic acid, capric acid, and lauric acid; omega-3 fatty acids such as hexadecatrienoic acid, α-linolenic acid, stearidonic acid, eicosatrienoic acid, eicosatetraenoic acid, eicosapentaenoic acid, heneicosapentaenoic acid, docosapentaenoic acid, clupanodonic acid, docosahexaenoic acid, tetracosapentaenoic acid, and tetracosahexaenoic acid; and omega-6 fatty acids such as linoleic acid, gamma-linolenic acid, calendic acid, eicosadienoic acid, dihomo-gamma-linolenic acid, arachidonic acid, docosadienoic acid, adrenic acid, osbond acid, tetracosatetraenoic acid, and tetracosapentaenoic acid.

According to the present invention, the term “organic polyol” refers to a linear, branched, or cyclic organic compound with 2 to 18 carbon atoms having at least three hydroxyl groups or having at least four hydroxyl groups. As such, the organic polyol may have 2, 3, 4, 5, 6, 7, 8, 9, 10, 11, 12, 13, 14, 15, 16, 17, or 18 carbon atoms. In one embodiment, no more than one hydroxyl group is connected to one carbon atom. In one embodiment, the organic polyol contains only carbon, hydrogen, and oxygen atoms.

According to the present invention, the term “at least three hydroxyl groups” means that the respective compound has three or more hydroxyl groups. In one embodiment, “at least three hydroxyl groups” includes 3 to 18 hydroxyl groups such as 3, 4, 5, 6, 7, 8, 9, 10, 11, 12, 13, 14, 15, 16, 17, or 18 hydroxyl groups. In one embodiment, “at least three hydroxyl groups” includes 3 to 12 hydroxyl groups such as 3, 4, 5, 6, 7, 8, 9, 10, 11, or 12 hydroxyl groups. In one embodiment, “at least three hydroxyl groups” includes 3 to 9 hydroxyl groups such as 3, 4, 5, 6, 7, 8, or 9 hydroxyl groups. In one embodiment, “at least three hydroxyl groups” includes 3 to 6 hydroxyl groups such as 3, 4, 5, or 6 hydroxyl groups.

According to the present invention, the term “at least four hydroxyl groups” means that the respective compound has four or more hydroxyl groups. In one embodiment, “at least four hydroxyl groups” includes 4 to 18 hydroxyl groups such as 4, 5, 6, 7, 8, 9, 10, 11, 12, 13, 14, 15, 16, 17, or 18 hydroxyl groups. In one embodiment, “at least four hydroxyl groups” includes 4 to 12 hydroxyl groups such as 4, 5, 6, 7, 8, 9, 10, 11, or 12 hydroxyl groups. In one embodiment, “at least four hydroxyl groups” includes 4 to 9 hydroxyl groups such as 4, 5, 6, 7, 8, or 9 hydroxyl groups. In one embodiment, “at least four hydroxyl groups” includes 4 to 6 hydroxyl groups such as 4, 5, or 6 hydroxyl groups.

According to the present invention, the term “leaving group” or “LG” refers to a group that departs with a pair of electrons in heterolytic bond cleavage. Exemplary leaving groups include halides (such as F, Cl, Br, or I), sulfonate esters (such as tosylate (TsO⁻)), pentafluoro phenolate, N-hydroxy succinimide, N,N-dicyclohexylurea, 1-hydroxy benzotriazole, 1-(3-(dimethylamino)propyl)-3-ethylurea, hydroxyl groups, ammonia groups and tertiary amines, thioesters, nitrates, phosphates, acetoacetates and carboxylates. In one embodiment, LG is acetoacetate, F, Cl, Br, I, or TsO.

It is to be understood that the linear or branched C₁₋₁₂ alkyl, C₃₋₈ cycloalkyl, linear or branched C₂₋₁₂ hydroxyalkyl, linear or branched C₁₋₁₂ carboxyalkly, phenyl, carboxyphenyl, and linear or branched and saturated or unsaturated C₁₋₂₄ alkanoyl may optionally be further substituted. Exemplary substituents include hydroxy, linear or branched C₁₋₁₂ alkyl, C₃₋₈ cycloalkyl, a carboxy group, halogen, and phenyl.

It is to be understood that if not explicitly stated otherwise, all stereoisomers, conformations and configurations are encompassed by compounds and functional groups which can be present as different stereoisomers or in different conformations and configurations. For example, the term “inositol” is to be understood as to include all stereoisomers and conformations such as myo-, scyllo-, muco-, D-chiro-, neo-inositol, L-chiro-, allo-, epi-, and cis-inositol. For example, the term “hexanetriol” is to be understood as to include all hexane isomers including three hydroxyl groups such as 1,1,1-hexanetriol, 1,1,2-hexanetriol, 1,2,2-hexanetriol, 1,2,3-hexanetriol, 1,2,4-hexanetriol, 1,2,5-hexanetriol, 1,2,6-hexanetriol, 1,3,5-hexanetriol, 1,3,6-hexanetriol, 2,3,4-hexanetriol, 2,3,5-hexanetriol etc.

The meanings and preferred meanings described herein for A, R, R¹, X and LG apply to all compounds and processes including the precursors of the compounds in any of the process steps detailed herein.

As used herein, the term “comprising” is to be construed as encompassing both “including” and “consisting of”, both meanings being specifically intended, and hence individually disclosed, embodiments according to the present invention.

As used herein, the articles “a” and “an” preceding an element or component are intended to be nonrestrictive regarding the number of instances (i.e. occurrences) of the element or component. Therefore, “a” or “an” is to be read to include one or at least one, and the singular word form of the element or component also includes the plural unless the number is obviously meant to be singular.

As used herein, the term “about” modifying the quantity of a substance, ingredient, component, or parameter employed refers to variation in the numerical quantity that can occur, for example, through typical measuring and handling procedures, e.g., liquid handling procedures used for making concentrates or solutions. Furthermore, variation can occur from inadvertent error in measuring procedures, differences in the manufacture, source, or purity of the ingredients employed to carry out the methods, and the like. In one embodiment, the term “about” means within 10% of the reported numerical value. In a more specific embodiment, the term “about” means within 5% of the reported numerical value.

As outlined above, subject of the present invention provides a compound of formula 1

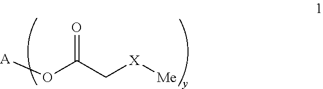

wherein
A is derived from an organic polyol with at least 4 hydroxyl groups,
X is —C(H)(OH)— or —C(H)(OR¹)—,
R¹ is selected from linear or branched C₁₋₁₂ alkyl, C₃₋₈ cycloalkyl, linear or branched C₂₋₁₂ hydroxyalkyl, linear or branched C₁₋₁₂ carboxyalkly, linear or branched and saturated or unsaturated C₁₋₂₄ alkanoyl, phenyl, and carboxyphenyl, and
y is from 1 to the number of hydroxyl groups of the initial organic polyol A.

In one embodiment the present invention provides a compound of formula 1

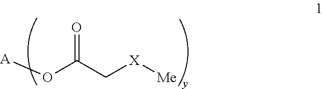

wherein
A is derived from an organic polyol with at least 4 hydroxyl groups,
X is —C(H)(OH)— or —C(H)(OR¹)—,
R¹ is selected from linear or branched C₁₋₁₂ alkyl, C₃₋₈ cycloalkyl, linear or branched C₂₋₁₂ hydroxyalkyl, linear or branched C₁₋₁₂ carboxyalkly, linear or branched and saturated or unsaturated C₁₋₂₄ alkanoyl, phenyl, and carboxyphenyl, and
y is from 4 to the number of hydroxyl groups of the initial organic polyol A.

In one embodiment, the organic polyol is a linear, branched, or cyclic organic compound with 2 to 18 carbon atoms having at least four hydroxyl groups.

In one embodiment, the organic polyol is selected from a linear or branched C₂₋₁₂ alkyl substituted with at least 4 hydroxyl groups or a C₃₋₈ cycloalkyl substituted with at least 4 hydroxyl groups.

Preferably, the linear or branched C₂₋₁₂ alkyl substituted with at least 4 hydroxyl groups is selected from the group consisting of pentaerythritol, butanetetrol, pentanetetrol, hexanetetrol, and hexanepentol.

Preferably, the C₃₋₈ cycloalkyl substituted with at least 4 hydroxyl groups is selected from the group consisting of cyclopentanetetrol, cyclopentanepentol, cyclohexanetetrol, cyclohexanepentol, and cyclohexanehexol.

In one embodiment, the organic polyol is selected from the group consisting of monosaccharides, sugar alcohols, and sugar acids.

Monosaccharides generally have the chemical formula CₙH₂ₙOₙ. Monosaccharides can be classified by the number x of carbon atoms they contain (CH₂O)ₓ: trioses (x=3), tetroses (x=4), pentoses (x=5), hexoses (x=6) and heptoses (x=7).

In one embodiment, the monosaccharide is selected from pentoses, hexoses, and heptoses.

Preferably, the monosaccharide is selected from aldopentoses, ketopentoses, aldohexosen, and ketohexoses.

In one embodiment, the monosaccharide is selected from the group consisting of ribose, arabinose, xylose, lyxose, ketopentose, ribulose, xylulose, allose, altrose, glucose, mannose, gulose, idose, galactose, talose, n-acetyl-d-glucosamin, glucosamin, N-acetyl-D-galactosamin, fucose, rhamnose, chinovose, fructose, 2-desoxy-D-glucose, fluordesoxyglucose, 6-desoxyfructose, 1,6-dichlorfructose, 3,6-anhydrogalactose, 1-O-methylgalactose, 1-O-methyl-D-glucose, 1-O-methyl-D-fructose, 3-O-methyl-D-fructose, 6-O-methyl-D-galactose, sedoheptulose, mannoheptulose, L-glycero-D-manno-heptose, and combinations thereof.

Sugar alcohols (also called polyhydric alcohols, polyalcohols, alditols or glycitols) are organic compounds, typically derived from sugars, containing one hydroxyl group (—OH) attached to each carbon atom.

In one embodiment, the sugar alcohol is selected from the group consisting of erythritol, threitol, arabitol, xylitol, ribitol, mannitol, sorbitol, galactitol, fucitol, iditol, inositol, volemitol, isomalt, maltitol, lactitol, and combinations thereof.

A sugar acid is generally a monosaccharide with a carboxyl group at one end or both ends of the carbon chain. Main classes of sugar acids include aldonic acids, ulosonic acids, uronic acids, and aldaric acids. In aldonic acids, the aldehyde group (—CHO) located at the initial end (position 1) of an aldose is oxidized. In ulosonic acids, the —CH₂(OH) group at the initial end of a 2-ketose is oxidized yielding an α-ketoacid. In uronic acids, the —CH₂(OH) group at the terminal end of an aldose or ketose is oxidized. In aldaric acids, both ends (—CHO and —CH₂(OH)) of an aldose are oxidized.

In one embodiment, the sugar acid is selected from aldonic acids, ulosonic acids, uronic acids, and aldaric acids. Preferably, the sugar acid is selected from the group consisting of xylonic acid, gluconic acid, ascorbic acid, neuraminic acid, ketodeoxyoctonic acid, glucuronic acid, galacturonic acid, iduronic acid, mucic acid, saccharic acid, and combinations thereof.

In one embodiment, the organic polyol is selected from the group consisting of sorbitol, xylitol, mannitol, erythritol, maltitol, glucose, glucitol, ribulose, and pentaerythritol. Preferably, the organic polyol is erythritol.

In one embodiment, y is from 2 to the number of hydroxyl groups of the initial organic polyol A. In one embodiment, y is from 3 to the number of hydroxyl groups of the initial organic polyol A. Preferably, y is from 4 to the number of hydroxyl groups of the initial organic polyol A. Accordingly, depending on the number of hydroxyl groups of the initial organic polyol A, y can be 1, 2, 3, 4, 5, 6, 7, 8, 9, or 10. In one embodiment, y is 2. In one embodiment, y is 3. In one embodiment, y is 4. In one embodiment, y is 5. In one embodiment, y is 6. In one embodiment, y is 7. In one embodiment, y is 8. In one embodiment, y is 9. In one embodiment, y is 10.

In one embodiment, in the compound according to formula 1, y is equal to the number of hydroxyl groups of the initial polyol A.

In one embodiment, the residues

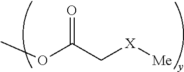

in the compound according to formula 1 may be identical or each independently different for each occurrence.

In one embodiment, the compound according to formula 1, all β-hydroxyl butyric acid ester units are either D-configured or L-configured. In another embodiment, all β-hydroxyl butyric acid ester units are present in the compound according to formula 1 as a non-racemic mixture of D- and L-configurations.

In one embodiment, the compound according to formula 1 contains more D-configured β-hydroxyl butyric acid ester units than L-configured β-hydroxyl butyric acid ester units. Preferably all β-hydroxyl butyric acid ester units are in D-configuration.

In one embodiment, in the compound according to formula 1, all β-hydroxyl butyric acid ester units are either R-configured or S-configured. In another embodiment, all β-hydroxyl butyric acid ester units are present in the compound according to formula 1 as a non-racemic mixture of R- and S-configurations.

In one embodiment, the compound according to formula 1 contains more R-configured β-hydroxyl butyric acid ester units than S-configured β-hydroxyl butyric acid ester units. Preferably all β-hydroxyl butyric acid ester units are in R-configuration.

In one embodiment, X is —C(H)(OH)—.

In one embodiment, the compound according to formula 1 is selected from the group consisting of

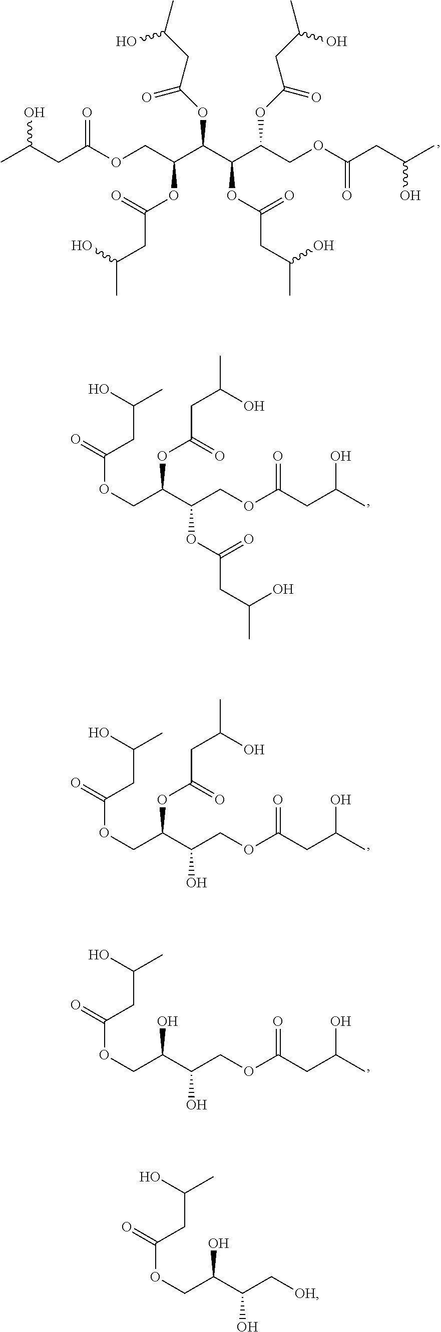 [SMILES: CC(O)CC(=O)OC[C@H](OC(=O)CC(C)O)[C@@H](OC(=O)CC(C)O)[C@H](OC(=O)CC(C)O)[C@@H](COC(=O)CC(C)O)OC(=O)CC(C)O ; CC(O)CC(=O)OC[C@H](OC(=O)CC(C)O)[C@@H](COC(=O)CC(C)O)OC(=O)CC(C)O ; CC(O)CC(=O)OC[C@H](O)[C@@H](COC(=O)CC(C)O)OC(=O)CC(C)O ; CC(O)CC(=O)OC[C@@H](O)[C@@H](O)COC(=O)CC(C)O ; CC(O)CC(=O)OC[C@@H](O)[C@@H](O)CO]

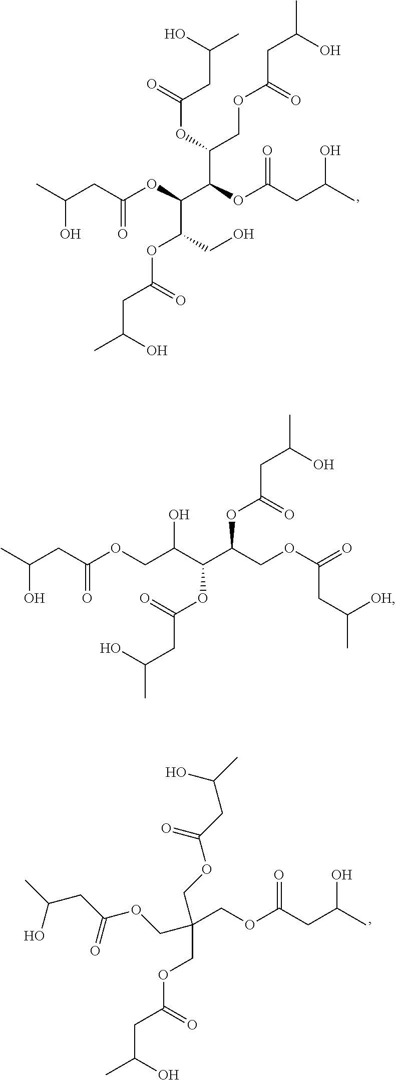

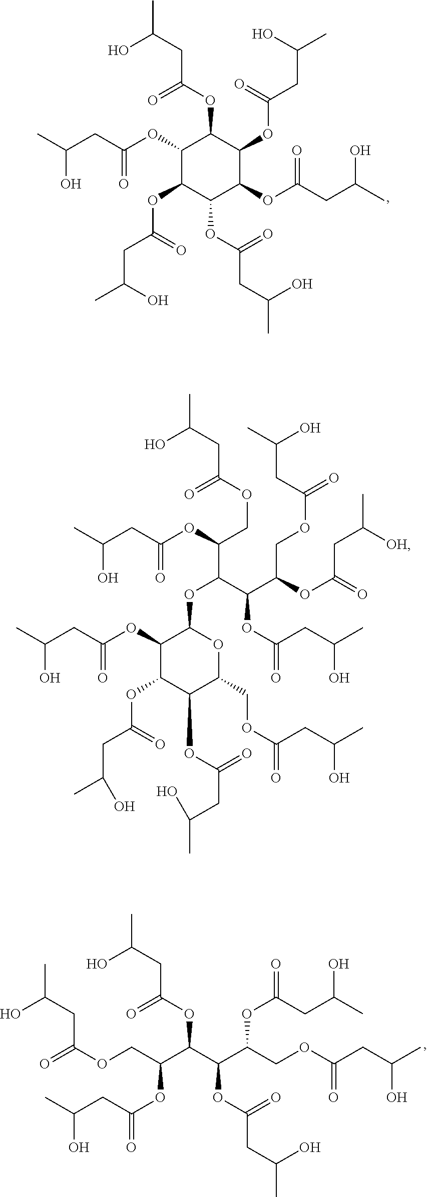

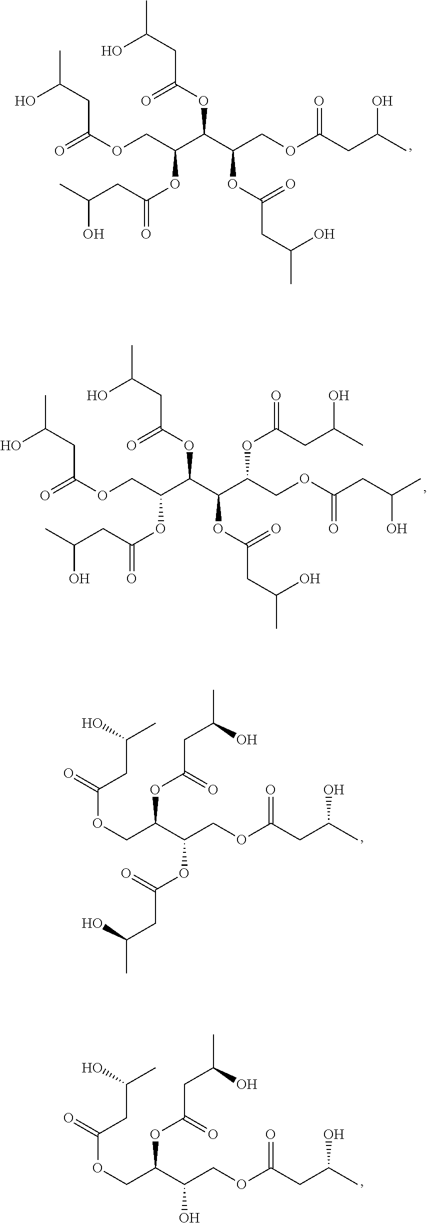 [SMILES: CC(O)CC(=O)OC[C@H](OC(=O)CC(C)O)[C@@H](OC(=O)CC(C)O)[C@@H](COC(=O)CC(C)O)OC(=O)CC(C)O ; CC(O)CC(=O)OC[C@@H](OC(=O)CC(C)O)[C@@H](OC(=O)CC(C)O)[C@H](OC(=O)CC(C)O)[C@@H](COC(=O)CC(C)O)OC(=O)CC(C)O ; C[C@@H](O)CC(=O)OC[C@H](OC(=O)C[C@@H](C)O)[C@@H](COC(=O)C[C@@H](C)O)OC(=O)C[C@@H](C)O ; C[C@@H](O)CC(=O)OC[C@H](O)[C@@H](COC(=O)C[C@@H](C)O)OC(=O)C[C@@H](C)O]

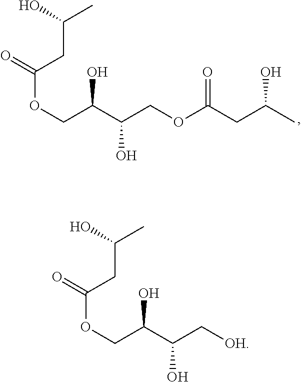 [SMILES: C[C@@H](O)CC(=O)OC[C@@H](O)[C@@H](O)COC(=O)C[C@@H](C)O ; C[C@@H](O)CC(=O)OC[C@@H](O)[C@@H](O)CO]

In another aspect, the present invention provides a process for the preparation of a compound of formula 1

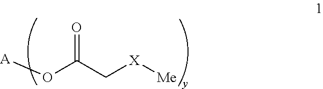

wherein
A is derived from an organic polyol with at least 4 hydroxyl groups,
X is —C(H)(OH)— or —C(H)(OR¹)—,
R¹ is selected from linear or branched C₁₋₁₂ alkyl, C₃₋₈ cycloalkyl, linear or branched C₂₋₁₂ hydroxyalkyl, linear or branched C₁₋₁₂ carboxyalkly, linear or branched and saturated or unsaturated C₁₋₂₄ alkanoyl, phenyl, and carboxyphenyl, and
y is from 1 to the number of hydroxyl groups of the initial organic polyol A;
wherein the process comprises:
   (i) reacting an organic polyol of formula 2 with diketene 3 resulting in the formation of a compound according to formula 4;

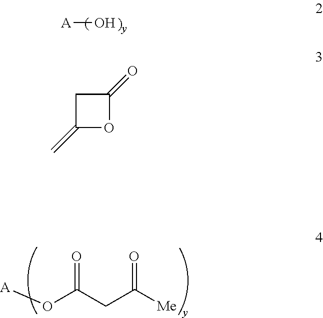

and
(iia) reacting the compound of formula 4 with hydrogen in the presence of a catalyst resulting in the formation of a compound according to formula 5,

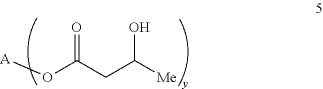

and optionally
(iib) reacting the compound of formula 5 with a compound LG-R¹, wherein LG is a leaving group, resulting in the formation of a compound according to formula 6;

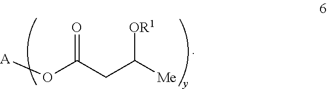

The inventors surprisingly found that the process according to the present invention for the preparation of compounds according to formula 1 achieves significantly improved atom economy and cost efficiency per unit of acetoacetate if diketene 3 is employed directly in this reaction. More BHB units or BHB derivate units per polyol core is favorable for applications in which a high ratio of or BHB units or derivatives thereof to the polyol is desired. Moreover, the terminal BHB units may be further reacted e.g. to BHB-esters. The process according to the present invention achieves a high BHB unit content per polyol unit. Moreover, the process according to the present invention provides BHB ester polyols in which the BHB units are further functionalized or protected, e.g. by an ester or ether.

In one embodiment, the present invention provides a process for the preparation of a compound of formula 1

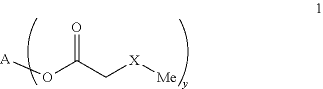

wherein
A is derived from an organic polyol with at least 4 hydroxyl groups,
X is —C(H)(OH)— or —C(H)(OR¹)—,
R¹ is selected from linear or branched C₁₋₁₂ alkyl, C₃₋₈ cycloalkyl, linear or branched C₂₋₁₂ hydroxyalkyl, linear or branched C₁₋₁₂ carboxyalkly, linear or branched and saturated or unsaturated C₁₋₂₄ alkanoyl, phenyl, and carboxyphenyl, and
y is from 4 to the number of hydroxyl groups of the initial organic polyol A;
wherein the process comprises:
   (i) reacting an organic polyol of formula 2 with diketene 3 resulting in the formation of a compound according to formula 4;

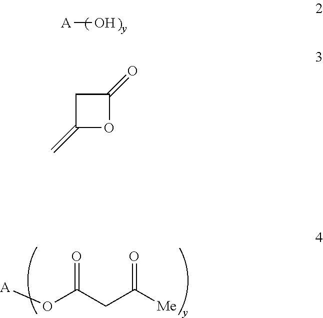

and
(iia) reacting the compound of formula 4 with hydrogen in the presence of a catalyst resulting in the formation of a compound according to formula 5,

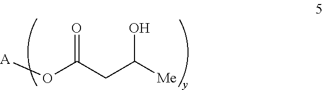

and optionally
(iib) reacting the compound of formula 5 with a compound LG-R¹, wherein LG is a leaving group, resulting in the formation of a compound according to formula 6;

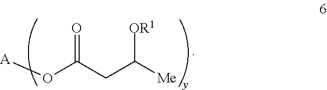

In one embodiment, the organic polyol is a linear, branched, or cyclic organic compound with 2 to 18 carbon atoms having at least four hydroxyl groups.

In one embodiment, the organic polyol is selected from a linear or branched C₂₋₁₂ alkyl substituted with at least 4 hydroxyl groups or a C₃₋₈ cycloalkyl substituted with at least 4 hydroxyl groups.

Preferably, the linear or branched C₂₋₁₂ alkyl substituted with at least 4 hydroxyl groups is selected from the group consisting of pentaerythritol, butanetetrol, pentanetetrol, hexanetetrol, and hexanepentol.

Preferably, the C₃₋₈ cycloalkyl substituted with at least 4 hydroxyl groups is selected from the group consisting of cyclopentanetetrol, cyclopentanepentol, cyclohexanetetrol, cyclohexanepentol, and cyclohexanehexol.

In one embodiment, the organic polyol is selected from the group consisting of monosaccharides, sugar alcohols, and sugar acids.

Monosaccharides generally have the chemical formula CₙH₂ₙOₙ. Monosaccharides can be classified by the number x of carbon atoms they contain (CH₂O)ₓ: trioses (x=3), tetroses (x=4), pentoses (x=5), hexoses (x=6) and heptoses (x=7).

In one embodiment, the monosaccharide is selected from pentoses, hexoses, and heptoses. Preferably, the monosaccharide is selected from aldopentoses, ketopentoses, aldohexosen, and ketohexoses.

In one embodiment, the monosaccharide is selected from the group consisting of ribose, arabinose, xylose, lyxose, ketopentose, ribulose, xylulose, allose, altrose, glucose, mannose, gulose, idose, galactose, talose, n-acetyl-d-glucosamin, glucosamin, N-acetyl-D-galactosamin, fucose, rhamnose, chinovose, fructose, 2-desoxy-D-glucose, fluordesoxyglucose, 6-desoxyfructose, 1,6-dichlorfructose, 3,6-anhydrogalactose, 1-O-methylgalactose, 1-O-methyl-D-glucose, 1-O-methyl-D-fructose, 3-O-methyl-D-fructose, 6-O-methyl-D-galactose, sedoheptulose, mannoheptulose, L-glycero-D-manno-heptose, and combinations thereof.

Sugar alcohols (also called polyhydric alcohols, polyalcohols, alditols or glycitols) are organic compounds, typically derived from sugars, containing one hydroxyl group (—OH) attached to each carbon atom.

In one embodiment, the sugar alcohol is selected from the group consisting of erythritol, threitol, arabitol, xylitol, ribitol, mannitol, sorbitol, galactitol, fucitol, iditol, inositol, volemitol, isomalt, maltitol, lactitol, and combinations thereof.

A sugar acid is generally a monosaccharide with a carboxyl group at one end or both ends of the carbon chain. Main classes of sugar acids include aldonic acids, ulosonic acids, uronic acids, and aldaric acids. In aldonic acids, the aldehyde group (—CHO) located at the initial end (position 1) of an aldose is oxidized. In ulosonic acids, the —CH₂(OH) group at the initial end of a 2-ketose is oxidized yielding an α-ketoacid. In uronic acids, the —CH₂(OH) group at the terminal end of an aldose or ketose is oxidized. In aldaric acids, both ends (—CHO and —CH₂(OH)) of an aldose are oxidized.

In one embodiment, the sugar acid is selected from aldonic acids, ulosonic acids, uronic acids, and aldaric acids. Preferably, the sugar acid is selected from the group consisting of xylonic acid, gluconic acid, ascorbic acid, neuraminic acid, ketodeoxyoctonic acid, glucuronic acid, galacturonic acid, iduronic acid, mucic acid, saccharic acid, and combinations thereof.

In one embodiment, the organic polyol is selected from the group consisting of sorbitol, xylitol, mannitol, erythritol, maltitol, glucose, glucitol, ribulose, and pentaerythritol. Preferably, the organic polyol is erythritol.

In one embodiment, y is from 2 to the number of hydroxyl groups of the initial organic polyol A. In one embodiment, y is from 3 to the number of hydroxyl groups of the initial organic polyol A. Preferably, y is from 4 to the number of hydroxyl groups of the initial organic polyol A. Accordingly, depending on the number of hydroxyl groups of the initial organic polyol A, y can be 1, 2, 3, 4, 5, 6, 7, 8, 9, or 10. In one embodiment, y is 2. In one embodiment, y is 3. In one embodiment, y is 4. In one embodiment, y is 5. In one embodiment, y is 6. In one embodiment, y is 7. In one embodiment, y is 8. In one embodiment, y is 9. In one embodiment, y is 10.

In one embodiment, in the compound according to formula 1, y is equal to the number of hydroxyl groups of the initial polyol A.

In one embodiment, the residues

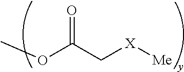

in the compound according to formula 1 may be identical or each independently different for each occurrence.

In one embodiment, the compound according to formula 1, all β-hydroxyl butyric acid ester units are either D-configured or L-configured. In another embodiment, all β-hydroxyl butyric acid ester units are present in the compound according to formula 1 as a non-racemic mixture of D- and L-configurations.

In one embodiment, the compound according to formula 1 contains more D-configured β-hydroxyl butyric acid ester units than L-configured β-hydroxyl butyric acid ester units. Preferably all β-hydroxyl butyric acid ester units are in D-configuration.

In one embodiment, in the compound according to formula 1, all β-hydroxyl butyric acid ester units are either R-configured or S-configured. In another embodiment, all β-hydroxyl butyric acid ester units are present in the compound according to formula 1 as a non-racemic mixture of R- and S-configurations.

In one embodiment, the compound according to formula 1 contains more R-configured β-hydroxyl butyric acid ester units than S-configured β-hydroxyl butyric acid ester units. Preferably all β-hydroxyl butyric acid ester units are in R-configuration.

In one embodiment, X is —C(H)(OH)—.

In one embodiment, the compound according to formula 1 is selected from the group consisting of

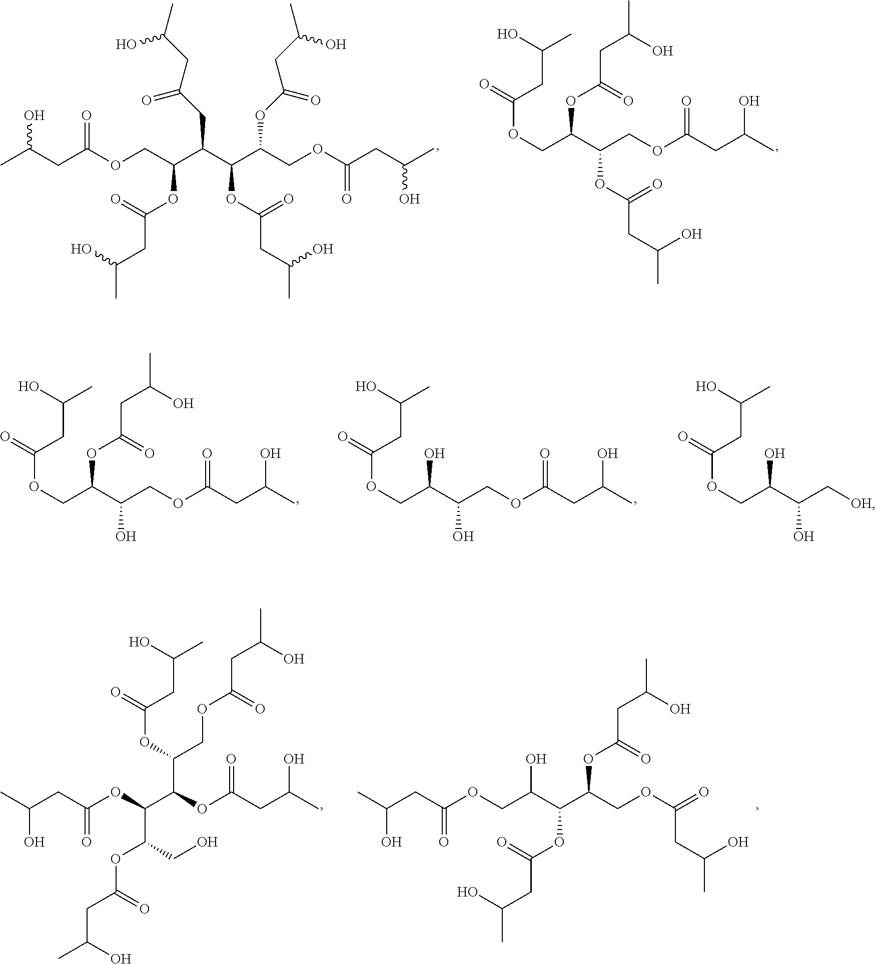

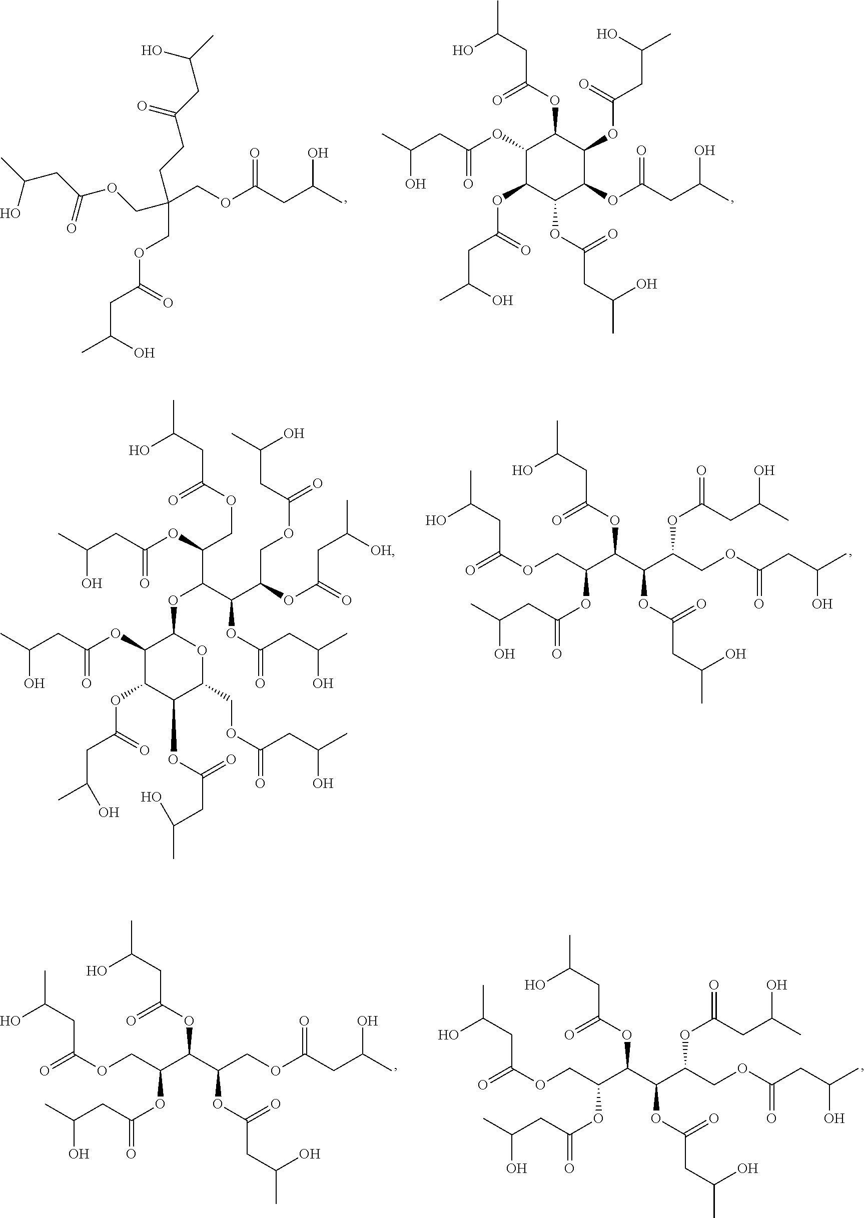

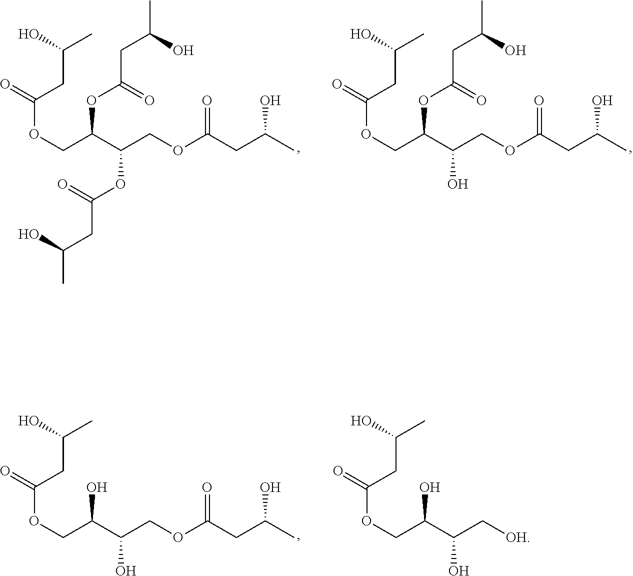 [SMILES: C[C@@H](O)CC(=O)OC[C@H](OC(=O)C[C@@H](C)O)[C@@H](COC(=O)C[C@@H](C)O)OC(=O)C[C@@H](C)O ; C[C@@H](O)CC(=O)OC[C@H](O)[C@@H](COC(=O)C[C@@H](C)O)OC(=O)C[C@@H](C)O ; C[C@@H](O)CC(=O)OC[C@@H](O)[C@@H](O)COC(=O)C[C@@H](C)O ; C[C@@H](O)CC(=O)OC[C@@H](O)[C@@H](O)CO]

In one embodiment, reaction step (i) is performed in the presence of an organic amine catalyst. Suitable organic amine catalysts include tertiary amines. Preferably, the organic amine catalyst is 1,4-diazabicyclo[2.2.2]octane (DABCO).

In step (iia) a compound of formula 4 is reacted with hydrogen in the presence of a catalyst resulting in the formation of a compound according to formula 5. In one embodiment, reaction step (iia) is performed in the presence of a metal-based catalyst. Preferably, the metal-based catalyst is a Ni-based catalyst, a Pd-based catalyst, a Pt-based catalyst, a Ru-based catalyst, a Co-based catalyst, an Ir-based catalyst, or an Rh-based catalyst.

In one embodiment, reaction step (iia) is performed in presence of a chiral ligand capable of forming complexes with the metal-based catalyst. Preferred chiral ligand are selected from the group consisting of 2,2′-bis(diphenylphosphino)-1,1′-binaphthyl (BINAP), 1,1′-Bi-2-naphthol (BINOL), 2,3-O-isopropylidene-2,3-dihydroxy-1,4-bis(diphenylphosphino)butane (DIOP), 2,2′,5,5′-tetramethyl-4,4′-bis-(diphenylphoshino)-3,3′-bithiophene (tetraMe-BITIOP), Bis(diphenylphosphino)-7,8-dihydro-6H-dibenzo[f,h][1,5]dioxonin (C3-TunePhos), 4,4′-Bis(bis(3,5-dimethylphenyl)phosphino)-2,2′,6,6′-tetramethoxy-3,3′-bipyridine (Xyl-p-PHOS), (6,6′-Dimethoxybiphenyl-2,2′-diyl)-bis-(diphenylphosphin) (MeO-BIPHEP), and 1,2-Bis[(2-methoxyphenyl)phenylphosphino]ethane (DIPAMP).

Preferably, reaction step (iia) is performed in the presence of a Ru-based catalyst. A preferred Ru-based catalyst is a Ruthenium oxide catalyst such as RuO₂. Further preferred Ru-based catalysts include Ru/C, RuAl₂O₃, Ru(OAc)₂(BINAP), Ru(Cl)₂(BINAP), C3-[(S,S)-teth-MtsDpenRuCl], [(R)-BinapRuCl(p-cymene)]Cl, and [Chloro(R)—C3-TunePhos)(p-cymene)ruthenium(II)]chloride.

In one embodiment, compound 5 is further esterified with an omega fatty acid, a medium-chain fatty acid, or a combination thereof at the hydroxyl group of at least one of the terminal β-hydroxyl butyric acid ester unit. In this context, it is to be understood that R¹ in the compound of formula 6 is a fatty acid residue derived from the fatty acids detailed herein. This is suitably done in reaction step (iib). In this case, LG-R¹ is used wherein R¹ is the fatty acid residue and LG the leaving group replacing the hydroxyl group at the carboxylic acid functionality, i.e. LG-C(O)—R— wherein R is a linear or branched and saturated or unsaturated C₁₋₂₄ alkyl residue. For example, LG-R¹ may be a fatty acid halide such as caproic acid chloride, caprylic acid chloride, capric acid chloride, or lauric acid chloride.

In one embodiment, the omega fatty acid is an omega-3 fatty acid, an omega-6 fatty acid, an omega-3,6 fatty acid, or a combination thereof. In one embodiment, the omega-3 fatty acid is selected from the group consisting of hexadecatrienoic acid, α-linolenic acid, stearidonic acid, eicosatrienoic acid, eicosatetraenoic acid, eicosapentaenoic acid, heneicosapentaenoic acid, docosapentaenoic acid, clupanodonic acid, docosahexaenoic acid, tetracosapentaenoic acid, and tetracosahexaenoic acid. In one embodiment, the omega-6 fatty acid is selected from the group consisting of linoleic acid, gamma-linolenic acid, calendic acid, eicosadienoic acid, dihomo-gamma-linolenic acid, arachidonic acid, docosadienoic acid, adrenic acid, osbond acid, tetracosatetraenoic acid, and tetracosapentaenoic acid.

In one embodiment, the medium-chain fatty acid is selected from the group consisting of caproic acid, caprylic acid, capric acid, lauric acid and combinations thereof.

In one embodiment, LG-R¹ is a fatty acid halide of any one of the fatty acids detailed herein.

Compound 5 may be esterified with only one species of omega fatty acids or medium-chain fatty acids or may be esterified with any combination of omega fatty acids and/or medium-chain fatty acids.

Depending on the type of the organic polyol, the process for the preparation of a compound of formula 1 may be performed in an organic solvent or without a solvent. Specifically, for liquid organic polyols or organic polyols having a low melting point (typically <120° C.), no organic solvent is necessary and the process can be performed without a solvent. Accordingly, in one embodiment, the process for the preparation of a compound of formula 1 is performed without a solvent. In another embodiment, the process for the preparation of a compound of formula 1 is performed in an organic solvent.

Suitable organic solvents include ethyl acetate, diethyl ether, MTBE, tetrahydrofurane, n-pentan, cyclopentan, n-Hexane, cyclohexane, n-heptan, DMF, DMSO, acetone, acetonitrile, toluene, chloroform, 1,4-dioxan, or o/m/p-xylene. Preferably, the organic solvent is ethyl acetate.

In one embodiment, in the process for the preparation of a compound of formula 1, reaction step (i) is performed at temperature of 20-100° C. Preferably, reaction step (i) is performed at temperature of 40-70° C. Additionally, the reaction temperature of reaction step (i) may be maintained at 40-70° C. after complete addition of diketene 3.

In one embodiment, in the process for the preparation of a compound of formula 1, reaction step (i) is performed at temperature of 0-100° C. Preferably, reaction step (i) is performed at temperature of 15-70° C. Additionally, the reaction temperature of reaction step (i) may be maintained at 20-70° C. after complete addition of diketene 3.

In one embodiment, during reaction step (i) diketene 3 is slowly added over a period of 1-6 h, e.g. dropwise, to the reaction mixture, to avoid the formation of side products.

In one embodiment, during reaction step (i) diketene 3 is slowly added over a period of 1-10 h, e.g. dropwise, to the reaction mixture, to avoid the formation of side products.

In one embodiment reaction step (iia) is performed in a closed vessel under hydrogen pressure. Preferably, reaction step (iia) is performed at 5-30 bar hydrogen pressure and even more preferably at 10-20 bar hydrogen pressure.

In one embodiment, reaction step (iia) is performed at a temperature of 20-90° C. In one embodiment, reaction step (iia) is performed at a temperature of 30-90° C. Preferably, reaction step (iia) is performed at a temperature of 50-70° C. and more preferably, reaction step (iia) is performed at a temperature of about 60° C.

In one embodiment, reaction step (iia) is stirred at 800-1200 rpm so as to ensure sufficient hydrogen diffusion into the reaction mixture.

In another aspect, the present invention provides a compound of formula 9

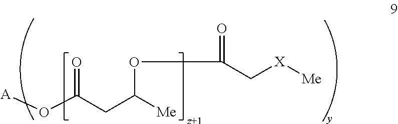

wherein
z is 0 or more,
A is derived from an organic polyol with at least 3 hydroxyl groups,
X is —C(O)—, —C(H)(OH)—, or —C(H)(OR¹)—,
R¹ is selected from linear or branched C₁₋₁₂ alkyl, C₃₋₈ cycloalkyl, linear or branched C₂₋₁₂ hydroxyalkyl, linear or branched C₁₋₁₂ carboxyalkly, linear or branched and saturated or unsaturated C₁₋₂₄ alkanoyl, phenyl, and carboxyphenyl, and
y is from 1 to the number of hydroxyl groups of the initial polyol A.

In one embodiment, the present invention provides a compound of formula 9

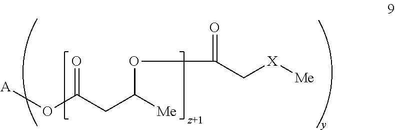

wherein
z is 0 or more,
A is derived from an organic polyol with at least 3 hydroxyl groups,
X is —C(O)—, —C(H)(OH)—, or —C(H)(OR¹)—,
R¹ is selected from linear or branched C₁₋₁₂ alkyl, C₃₋₈ cycloalkyl, linear or branched C₂₋₁₂ hydroxyalkyl, linear or branched C₁₋₁₂ carboxyalkly, linear or branched and saturated or unsaturated C₁₋₂₄ alkanoyl, phenyl, and carboxyphenyl, and
y is from 3 to the number of hydroxyl groups of the initial polyol A.

In one embodiment, z is from 0-100 such as from 0-95, 0-90, 0-85, 0-80, 0-75, 0-70, 0-65, 0-60, 0-55, 0-50, 0-45, 0-40, 0-35, 0-30, 0-25, or 0-20. In one embodiment, z is from 0-20 such as 0, 1, 2, 3, 4, 5, 6, 7, 8, 9, 10, 11, 12, 13, 14, 15, 16, 17, 18, 19, or 20. In one embodiment, z is from 0-20, such as 0-19, 0-18, 0-17, 0-16, 0-15, 0-14, 0-13, 0-12, 0-11, 0-10, 0-9, 0-8, 0-7, 0-6, 0-5, 0-4, 0-3, 0-2, 1, or 0. Preferably, z is 0 or 1.

In one embodiment, the organic polyol is a linear, branched, or cyclic organic compound with 2 to 18 carbon atoms having at least three hydroxyl groups.

In one embodiment, the organic polyol is selected from a linear or branched C₂₋₁₂ alkyl substituted with at least 3 hydroxyl groups or a C₃₋₈ cycloalkyl substituted with at least 3 hydroxyl groups.

Preferably, the linear or branched C₂₋₁₂ alkyl substituted with at least 3 hydroxyl groups is selected from the group consisting of glycerol, trimethylolpropane, butanetriol, 2-methyl-propanetriol, pentanetriol, 3-methyl-pentanetriol, hexanetriol, pentaerythritol, butanetetrol, pentanetetrol, hexanetetrol, hexanepentol, and combinations thereof.

In one embodiment, the C₃₋₈ cycloalkyl substituted with at least 3 hydroxyl groups is selected from the group consisting of cyclopentanetriol, cyclohexanetriol, cyclopentanetetrol, cyclohexanetetrol, and combinations thereof.

In one embodiment, the organic polyol is selected from the group consisting of monosaccharides, sugar alcohols, and sugar acids.

In one embodiment, the monosaccharide is selected from tetroses, pentoses, hexoses, and heptoses preferably wherein the monosaccharide is selected from aldotetroses, ketotetroses, aldopentoses, ketopentoses, aldohexosen, ketohexoses, aldoheptoses and ketoheptoses.

Preferably, the monosaccharide is selected from the group consisting of erythrose, threose, erythrulose, ribose, arabinose, xylose, lyxose, desoxyribose, ketopentose, ribulose, xylulose, allose, altrose, glucose, mannose, gulose, idose, galactose, talose, n-acetyl-d-glucosamin, glucosamin, N-acetyl-D-galactosamin, fucose, rhamnose, chinovose, fructose, 2-desoxy-D-glucose, fluordesoxyglucose, 6-desoxyfructose, 1,6-dichlorfructose, 3,6-anhydrogalactose, 1-O-methylgalactose, 1-O-methyl-D-glucose, 1-O-methyl-D-fructose, 3-O-methyl-D-fructose, 6-O-methyl-D-galactose, sedoheptulose, mannoheptulose, L-glycero-D-manno-heptose, and combinations thereof.

Preferably, the sugar alcohol is selected from the group consisting of erythritol, threitol, arabitol, xylitol, ribitol, mannitol, sorbitol, galactitol, fucitol, iditol, inositol, volemitol, isomalt, maltitol, lactitol, and combinations thereof.

Preferably, the sugar acid is selected from the group consisting of xylonic acid, gluconic acid, ascorbic acid, neuraminic acid, ketodeoxyoctonic acid, glucuronic acid, galacturonic acid, iduronic acid, mucic acid, saccharic acid, and combinations thereof.

In one embodiment, the organic polyol is selected from the group consisting of glycerol, sorbitol, xylitol, mannitol, erythritol, maltitol, glucose, glucitol, ribulose, pentaerythritol, and trimethylolpropane. Preferably, the organic polyol is erythritol.

In one embodiment, y is from 2 to the number of hydroxyl groups of the initial organic polyol A. In one embodiment, y is from 3 to the number of hydroxyl groups of the initial organic polyol A. In one embodiment, y is from 4 to the number of hydroxyl groups of the initial organic polyol A. Accordingly, depending on the number of hydroxyl groups of the initial organic polyol A, y can be 1, 2, 3, 4, 5, 6, 7, 8, 9, or 10. In one embodiment, y is 2. In one embodiment, y is 3. In one embodiment, y is 4. In one embodiment, y is 5. In one embodiment, y is 6. In one embodiment, y is 7. In one embodiment, y is 8. In one embodiment, y is 9. In one embodiment, y is 10.

In one embodiment, in the compound according to formula 9, y is equal to the number of hydroxyl groups of the initial polyol A.

In one embodiment, the residues

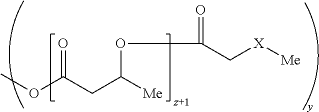

in the compound according to formula 9 may be identical or each independently different for each occurrence.

In one embodiment, the compound according to formula 9, all β-hydroxyl butyric acid ester units are either D-configured or L-configured. In another embodiment, all β-hydroxyl butyric acid ester units are present in the compound according to formula 9 as a non-racemic mixture of D- and L-configurations.

In one embodiment, the compound according to formula 9 contains more D-configured β-hydroxyl butyric acid ester units than L-configured β-hydroxyl butyric acid ester units. Preferably all β-hydroxyl butyric acid ester units are in D-configuration.

In one embodiment, in the compound according to formula 9, all β-hydroxyl butyric acid ester units are either R-configured or S-configured. In another embodiment, all β-hydroxyl butyric acid ester units are present in the compound according to formula 9 as a non-racemic mixture of R- and S-configurations.

In one embodiment, the compound according to formula 9 contains more R-configured β-hydroxyl butyric acid ester units than S-configured β-hydroxyl butyric acid ester units. Preferably all β-hydroxyl butyric acid ester units are in R-configuration.

In one embodiment, X is —C(O)—. In one embodiment, X is —C(H)(OH)—.

In one embodiment, the compound according to formula 9 is selected from the group consisting of

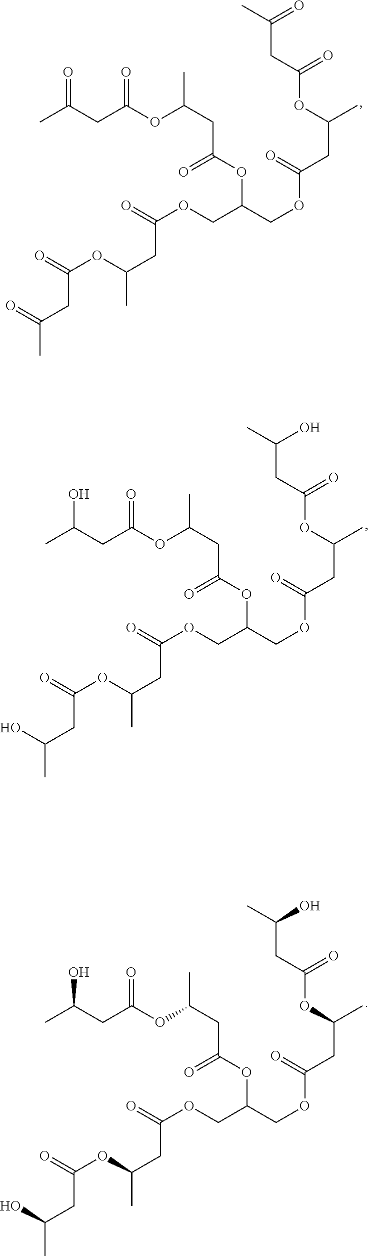 [SMILES: CC(=O)CC(=O)OC(C)CC(=O)OCC(COC(=O)CC(C)OC(=O)CC(C)=O)OC(=O)CC(C)OC(=O)CC(C)=O ; CC(O)CC(=O)OC(C)CC(=O)OCC(COC(=O)CC(C)OC(=O)CC(C)O)OC(=O)CC(C)OC(=O)CC(C)O ; C[C@H](CC(=O)OCC(COC(=O)C[C@H](C)OC(=O)C[C@@H](C)O)OC(=O)C[C@@H](C)OC(=O)C[C@@H](C)O)OC(=O)C[C@@H](C)O]

In another aspect, the present invention provides a process for the preparation of a compound of formula 9

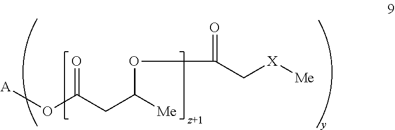

wherein
z is 0 or more,
A is derived from an organic polyol with at least 3 hydroxyl groups,
X is —C(O)—, —C(H)(OH)—, or —C(H)(OR¹)—,
R¹ is selected from linear or branched C₁₋₁₂ alkyl, C₃₋₈ cycloalkyl, linear or branched C₂₋₁₂ hydroxyalkyl, linear or branched C₁₋₁₂ carboxyalkly, linear or branched and saturated or unsaturated C₁₋₂₄ alkanoyl, phenyl, and carboxyphenyl, and
y is from 1 to the number of hydroxyl groups of the initial organic polyol A; wherein the process comprises:
   (i) reacting a compound of formula 10 with diketene 3 resulting in the formation of a compound according to formula 11;

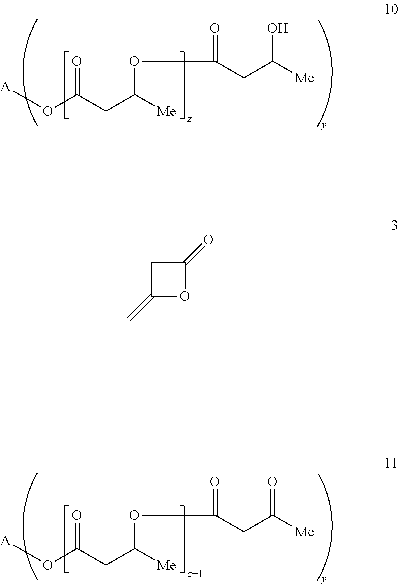

optionally
(iia) reacting the compound of formula 11 with hydrogen in the presence of a catalyst resulting in the formation of a compound according to formula 12,

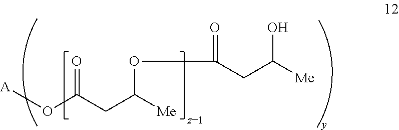

and optionally
(iib) reacting the compound of formula 12 with a compound LG-R¹, wherein LG is a leaving group, resulting in the formation of a compound according to formula 13;

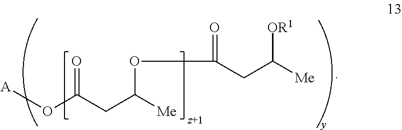

In one embodiment, the present invention provides a process for the preparation of a compound of formula 9

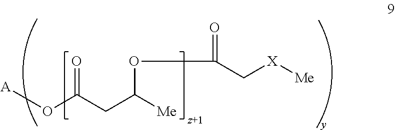

wherein
z is 0 or more,
A is derived from an organic polyol with at least 3 hydroxyl groups,
X is —C(O)—, —C(H)(OH)—, or —C(H)(OR¹)—,
R¹ is selected from linear or branched C₁₋₁₂ alkyl, C₃₋₈ cycloalkyl, linear or branched C₂₋₁₂ hydroxyalkyl, linear or branched C₁₋₁₂ carboxyalkly, linear or branched and saturated or unsaturated C₁₋₂₄ alkanoyl, phenyl, and carboxyphenyl, and
y is from 3 to the number of hydroxyl groups of the initial organic polyol A;
wherein the process comprises:
   (i) reacting a compound of formula 10 with diketene 3 resulting in the formation of a compound according to formula 11;

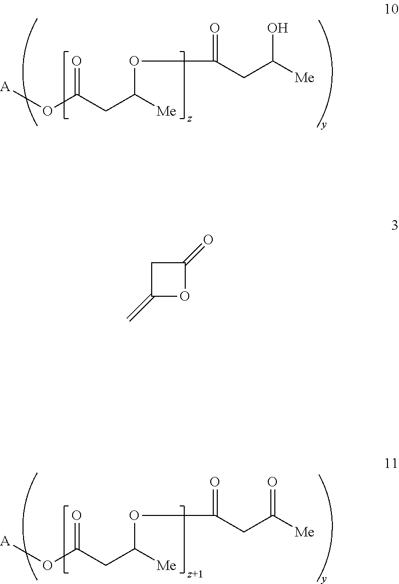

optionally
(iia) reacting the compound of formula 11 with hydrogen in the presence of a catalyst resulting in the formation of a compound according to formula 12,

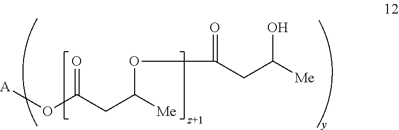

and optionally
(iib) reacting the compound of formula 12 with a compound LG-R¹, wherein LG is a leaving group, resulting in the formation of a compound according to formula 13;

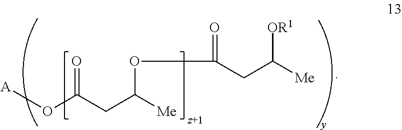

The inventors surprisingly found that the process according to the present invention for the preparation of a compound of formula 9 achieves significantly improved atom economy and cost efficiency per unit of acetoacetate if a compound according to formula 10 is reacted with diketene 3 resulting in the formation of a compound according to formula 11. More acetoacetate and/or BHB units per polyol core is favorable for applications in which a high ratio of acetoacetate and/or BHB units or derivatives thereof to the polyol is desired. Moreover, the inventors surprisingly found that after hydrogenation of compound 11 to compound 12, the process of reacting the obtained compound with diketene 3 may be repeated. This ultimately yields dendrimers with multiple BHB units of a desired length. The terminal acetoacetate and/or BHB units may be further reacted e.g. to BHB-esters. Thus, the process according to the present invention achieves a high BHB unit content per polyol unit. Moreover, the process according to the present invention provides BHB ester polyols in which the BHB units are further functionalized or protected, e.g. by an ester or ether.

In one embodiment, z is from 0-100 such as from 0-95, 0-90, 0-85, 0-80, 0-75, 0-70, 0-65, 0-60, 0-55, 0-50, 0-45, 0-40, 0-35, 0-30, 0-25, or 0-20. In one embodiment, z is from 0-20 such as 0, 1, 2, 3, 4, 5, 6, 7, 8, 9, 10, 11, 12, 13, 14, 15, 16, 17, 18, 19, or 20. In one embodiment, z is from 0-20, such as 0-19, 0-18, 0-17, 0-16, 0-15, 0-14, 0-13, 0-12, 0-11, 0-10, 0-9, 0-8, 0-7, 0-6, 0-5, 0-4, 0-3, 0-2, 1, or 0. Preferably, z is 0 or 1.

In one embodiment, the organic polyol is a linear, branched, or cyclic organic compound with 2 to 18 carbon atoms having at least three hydroxyl groups.

In one embodiment, the organic polyol is selected from a linear or branched C₂₋₁₂ alkyl substituted with at least 3 hydroxyl groups or a C₃₋₈ cycloalkyl substituted with at least 3 hydroxyl groups.

Preferably, the linear or branched C₂₋₁₂ alkyl substituted with at least 3 hydroxyl groups is selected from the group consisting of glycerol, trimethylolpropane, butanetriol, 2-methyl-propanetriol, pentanetriol, 3-methyl-pentanetriol, hexanetriol, pentaerythritol, butanetetrol, pentanetetrol, hexanetetrol, hexanepentol, and combinations thereof.

In one embodiment, the C₃₋₈ cycloalkyl substituted with at least 3 hydroxyl groups is selected from the group consisting of cyclopentanetriol, cyclohexanetriol, cyclopentanetetrol, cyclohexanetetrol, and combinations thereof.

In one embodiment, the organic polyol is selected from the group consisting of monosaccharides, sugar alcohols, and sugar acids.

In one embodiment, the monosaccharide is selected from tetroses, pentoses, hexoses, and heptoses preferably wherein the monosaccharide is selected from aldotetroses, ketotetroses, aldopentoses, ketopentoses, aldohexosen, ketohexoses, aldoheptoses and ketoheptoses.

Preferably, the monosaccharide is selected from the group consisting of erythrose, threose, erythrulose, ribose, arabinose, xylose, lyxose, desoxyribose, ketopentose, ribulose, xylulose, allose, altrose, glucose, mannose, gulose, idose, galactose, talose, n-acetyl-d-glucosamin, glucosamin, N-acetyl-D-galactosamin, fucose, rhamnose, chinovose, fructose, 2-desoxy-D-glucose, fluordesoxyglucose, 6-desoxyfructose, 1,6-dichlorfructose, 3,6-anhydrogalactose, 1-O-methylgalactose, 1-O-methyl-D-glucose, 1-O-methyl-D-fructose, 3-O-methyl-D-fructose, 6-O-methyl-D-galactose, sedoheptulose, mannoheptulose, L-glycero-D-manno-heptose, and combinations thereof.

Preferably, the sugar alcohol is selected from the group consisting of erythritol, threitol, arabitol, xylitol, ribitol, mannitol, sorbitol, galactitol, fucitol, iditol, inositol, volemitol, isomalt, maltitol, lactitol, and combinations thereof.

Preferably, the sugar acid is selected from the group consisting of xylonic acid, gluconic acid, ascorbic acid, neuraminic acid, ketodeoxyoctonic acid, glucuronic acid, galacturonic acid, iduronic acid, mucic acid, saccharic acid, and combinations thereof.

In one embodiment, the organic polyol is selected from the group consisting of glycerol, sorbitol, xylitol, mannitol, erythritol, maltitol, glucose, glucitol, ribulose, pentaerythritol, and trimethylolpropane. Preferably, the organic polyol is erythritol.

In one embodiment, y is from 2 to the number of hydroxyl groups of the initial organic polyol A. In one embodiment, y is from 3 to the number of hydroxyl groups of the initial organic polyol A. In one embodiment, y is from 4 to the number of hydroxyl groups of the initial organic polyol A. Accordingly, depending on the number of hydroxyl groups of the initial organic polyol A, y can be 1, 2, 3, 4, 5, 6, 7, 8, 9, or 10. In one embodiment, y is 2. In one embodiment, y is 3. In one embodiment, y is 4. In one embodiment, y is 5. In one embodiment, y is 6. In one embodiment, y is 7. In one embodiment, y is 8.

In one embodiment, y is 9. In one embodiment, y is 10.

In one embodiment, in the compound according to formula 9, y is equal to the number of hydroxyl groups of the initial polyol A.

In one embodiment, the residues

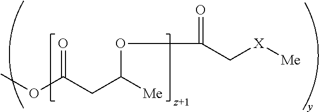

in the compound according to formula 9 may be identical or each independently different for each occurrence.

In one embodiment, the compound according to formula 9, all β-hydroxyl butyric acid ester units are either D-configured or L-configured. In another embodiment, all β-hydroxyl butyric acid ester units are present in the compound according to formula 9 as a non-racemic mixture of D- and L-configurations.

In one embodiment, the compound according to formula 9 contains more D-configured β-hydroxyl butyric acid ester units than L-configured β-hydroxyl butyric acid ester units. Preferably all β-hydroxyl butyric acid ester units are in D-configuration.

In one embodiment, in the compound according to formula 9, all β-hydroxyl butyric acid ester units are either R-configured or S-configured. In another embodiment, all β-hydroxyl butyric acid ester units are present in the compound according to formula 9 as a non-racemic mixture of R- and S-configurations.

In one embodiment, the compound according to formula 9 contains more R-configured β-hydroxyl butyric acid ester units than S-configured β-hydroxyl butyric acid ester units. Preferably all β-hydroxyl butyric acid ester units are in R-configuration.

In one embodiment, X is —C(O)—. In one embodiment, X is —C(H)(OH)—.

In one embodiment, the compound according to formula 9 is selected from the group consisting of

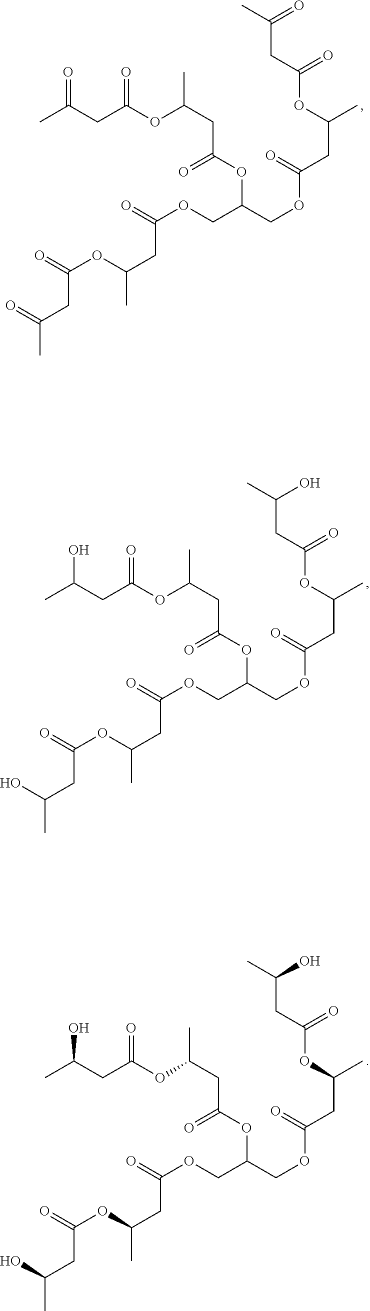 [SMILES: CC(=O)CC(=O)OC(C)CC(=O)OCC(COC(=O)CC(C)OC(=O)CC(C)=O)OC(=O)CC(C)OC(=O)CC(C)=O ; CC(O)CC(=O)OC(C)CC(=O)OCC(COC(=O)CC(C)OC(=O)CC(C)O)OC(=O)CC(C)OC(=O)CC(C)O ; C[C@H](CC(=O)OCC(COC(=O)C[C@H](C)OC(=O)C[C@@H](C)O)OC(=O)C[C@@H](C)OC(=O)C[C@@H](C)O)OC(=O)C[C@@H](C)O]

In one embodiment, reaction step (i) is performed in the presence of an organic amine catalyst. Suitable organic amine catalysts include tertiary amines. Preferably, the organic amine catalyst is 1,4-diazabicyclo[2.2.2]octane (DABCO).

In step (iia) a compound of formula 11 is reacted with hydrogen in the presence of a catalyst resulting in the formation of a compound according to formula 12. In one embodiment, reaction step (iia) is performed in the presence of a metal-based catalyst. Preferably, the metal-based catalyst is a Ni-based catalyst, a Pd-based catalyst, a Pt-based catalyst, a Ru-based catalyst, a Co-based catalyst, an Ir-based catalyst, or a Rh-based catalyst.

In one embodiment, reaction step (iia) is performed in presence of a chiral ligand capable of forming complexes with the metal-based catalyst. Preferred chiral ligand are selected from the group consisting of 2,2′-bis(diphenylphosphino)-1,1′-binaphthyl (BINAP), 1,1′-Bi-2-naphthol (BINOL), 2,3-O-isopropylidene-2,3-dihydroxy-1,4-bis(diphenylphosphino)butane (DIOP), 2,2′,5,5′-tetramethyl-4,4′-bis-(diphenylphoshino)-3,3′-bithiophene (tetraMe-BITIOP), Bis(diphenylphosphino)-7,8-dihydro-6H-dibenzo[f,h][1,5]dioxonin (C3-TunePhos), 4,4′-Bis(bis(3,5-dimethylphenyl)phosphino)-2,2′,6,6′-tetramethoxy-3,3′-bipyridine (Xyl-p-PHOS), (6,6′-Dimethoxybiphenyl-2,2′-diyl)-bis-(diphenylphosphin) (MeO-BIPHEP), and 1,2-Bis[(2-methoxyphenyl)phenylphosphino]ethane (DIPAMP).

Preferably, reaction step (iia) is performed in the presence of a Ru-based catalyst. A preferred Ru-based catalyst is either a Ruthenium oxide catalyst such as RuO₂. Further preferred Ru-based catalysts include Ru/C, RuAl₂O₃, Ru(OAc)₂(BINAP), Ru(Cl)₂(BINAP), C3-[(S,S)-teth-MtsDpenRuCl], [(R)-BinapRuCl(p-cymene)]Cl, and [Chloro(R)—C3-TunePhos)(p-cymene)ruthenium(II)]chloride.

In one embodiment, compound 12 is further esterified with an omega fatty acid, a medium-chain fatty acid, or a combination thereof at the hydroxyl group of at least one of the terminal β-hydroxyl butyric acid ester unit. In this context, it is to be understood that R¹ in the compound of formula 13 is a fatty acid residue derived from the fatty acids detailed herein. This is suitably done in reaction step (iib). In this case, LG-R¹ is used wherein R¹ is the fatty acid residue and LG the leaving group replacing the hydroxyl group at the carboxylic acid functionality, i.e. LG-C(O)—R— wherein R is a linear or branched and saturated or unsaturated C₁₋₂₄ alkyl residue). For example, LG-R¹ may be a fatty acid halide such as caproic acid chloride, caprylic acid chloride, capric acid chloride, or lauric acid chloride.

In one embodiment, the omega fatty acid is an omega-3 fatty acid, an omega-6 fatty acid, an omega-3,6 fatty acid, or a combination thereof. In one embodiment, the omega-3 fatty acid is selected from the group consisting of hexadecatrienoic acid, α-linolenic acid, stearidonic acid, eicosatrienoic acid, eicosatetraenoic acid, eicosapentaenoic acid, heneicosapentaenoic acid, docosapentaenoic acid, clupanodonic acid, docosahexaenoic acid, tetracosapentaenoic acid, and tetracosahexaenoic acid. In one embodiment, the omega-6 fatty acid is selected from the group consisting of linoleic acid, gamma-linolenic acid, calendic acid, eicosadienoic acid, dihomo-gamma-linolenic acid, arachidonic acid, docosadienoic acid, adrenic acid, osbond acid, tetracosatetraenoic acid, and tetracosapentaenoic acid.

In one embodiment, the medium-chain fatty acid is selected from the group consisting of caproic acid, caprylic acid, capric acid, lauric acid and combinations thereof.

In one embodiment, LG-R¹ is a fatty acid halide of any one of the fatty acids detailed herein.

Compound 12 may be esterified with only one species of omega fatty acids or medium-chain fatty acids or may be esterified with any combination of omega fatty acids and/or medium-chain fatty acids.

Depending on the type of the organic polyol, the process for the preparation of a compound of formula 9 may be performed in an organic solvent or without a solvent. Specifically, for liquid organic polyols or organic polyols having a low melting point (typically <120° C.), no organic solvent is necessary and the process can be performed without a solvent. Accordingly, in one embodiment, the process for the preparation of a compound of formula 9 is performed without a solvent. In another embodiment, the process for the preparation of a compound of formula 9 is performed in an organic solvent.

Suitable organic solvents include ethyl acetate, diethyl ether, MTBE, tetrahydrofurane, n-pentan, cyclopentan, n-Hexane, cyclohexane, n-heptan, DMF, DMSO, acetone, t-butyl alcohol, acetonitrile, toluene, chloroform, 1,4-dioxan, methanol, ethanol, or o/m/p-xylene. Preferably, the organic solvent is ethyl acetate.

In one embodiment, in the process for the preparation of a compound of formula 9, reaction step (i) is performed at temperature of 20-100° C. Preferably, reaction step (i) is performed at temperature of 40-70° C. Additionally, the reaction temperature of reaction step (i) may be maintained at 40-70° C. after complete addition of diketene 3.

In one embodiment, in the process for the preparation of a compound of formula 9, reaction step (i) is performed at temperature of 0-100° C. Preferably, reaction step (i) is performed at temperature of 15-70° C. Additionally, the reaction temperature of reaction step (i) may be maintained at 20-70° C. after complete addition of diketene 3.

In one embodiment, during reaction step (i) diketene 3 is slowly added over a period of 1-6 h, e.g. dropwise, to the reaction mixture, to avoid the formation of side products.

In one embodiment, during reaction step (i) diketene 3 is slowly added over a period of 1-10 h, e.g. dropwise, to the reaction mixture, to avoid the formation of side products.

In one embodiment reaction step (iia) is performed in a closed vessel under hydrogen pressure. Preferably, reaction step (iia) is performed at 5-30 bar hydrogen pressure and even more preferably at 10-20 bar hydrogen pressure.

In one embodiment, reaction step (iia) is performed at a temperature of 20-90° C. In one embodiment, reaction step (iia) is performed at a temperature of 30-90° C. Preferably, reaction step (iia) is performed at a temperature of 50-70° C. and more preferably, reaction step (iia) is performed at a temperature of about 60° C.

In one embodiment, reaction step (iia) is stirred at 800-1200 rpm so as to ensure sufficient hydrogen diffusion into the reaction mixture.

In another aspect, the present invention provides a compound of formula 9

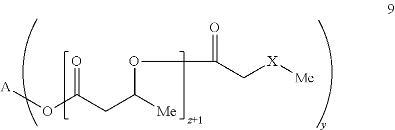

wherein
z is 0 or 1,
A is derived from an organic polyol with at least 3 hydroxyl groups,
X is —C(O)—, —C(H)(OH)—, or —C(H)(OR¹)—,
R¹ is selected from linear or branched C₁₋₁₂ alkyl, C₃₋₈ cycloalkyl, linear or branched C₂₋₁₂ hydroxyalkyl, linear or branched C₁₋₁₂ carboxyalkly, linear or branched and saturated or unsaturated C₁₋₂₄ alkanoyl, phenyl, and carboxyphenyl, and
y is from 1 to the number of hydroxyl groups of the initial polyol A.

In one embodiment, the present invention provides a compound of formula 9

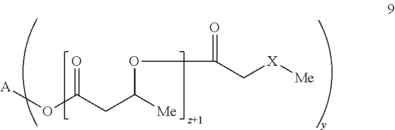

wherein
z is 0 or 1,
A is derived from an organic polyol with at least 3 hydroxyl groups,
X is —C(O)—, —C(H)(OH)—, or —C(H)(OR¹)—, R¹ is selected from linear or branched C₁₋₁₂ alkyl, C₃₋₈ cycloalkyl, linear or branched C₂₋₁₂ hydroxyalkyl, linear or branched C₁₋₁₂ carboxyalkly, linear or branched and saturated or unsaturated C₁₋₂₄ alkanoyl, phenyl, and carboxyphenyl, and
y is from 3 to the number of hydroxyl groups of the initial polyol A.

In one embodiment, the organic polyol is a linear, branched, or cyclic organic compound with 2 to 18 carbon atoms having at least three hydroxyl groups.

In one embodiment, the organic polyol is selected from a linear or branched C₂₋₁₂ alkyl substituted with at least 3 hydroxyl groups or a C₃₋₈ cycloalkyl substituted with at least 3 hydroxyl groups.

Preferably, the linear or branched C₂₋₁₂ alkyl substituted with at least 3 hydroxyl groups is selected from the group consisting of glycerol, trimethylolpropane, butanetriol, 2-methyl-propanetriol, pentanetriol, 3-methyl-pentanetriol, hexanetriol, pentaerythritol, butanetetrol, pentanetetrol, hexanetetrol, hexanepentol, and combinations thereof.

In one embodiment, the C₃₋₈ cycloalkyl substituted with at least 3 hydroxyl groups is selected from the group consisting of cyclopentanetriol, cyclohexanetriol, cyclopentanetetrol, cyclohexanetetrol, and combinations thereof.

In one embodiment, the organic polyol is selected from the group consisting of monosaccharides, sugar alcohols, and sugar acids.

In one embodiment, the monosaccharide is selected from tetroses, pentoses, hexoses, and heptoses preferably wherein the monosaccharide is selected from aldotetroses, ketotetroses, aldopentoses, ketopentoses, aldohexosen, ketohexoses, aldoheptoses and ketoheptoses.

Preferably, the monosaccharide is selected from the group consisting of erythrose, threose, erythrulose, ribose, arabinose, xylose, lyxose, desoxyribose, ketopentose, ribulose, xylulose, allose, altrose, glucose, mannose, gulose, idose, galactose, talose, n-acetyl-d-glucosamin, glucosamin, N-acetyl-D-galactosamin, fucose, rhamnose, chinovose, fructose, 2-desoxy-D-glucose, fluordesoxyglucose, 6-desoxyfructose, 1,6-dichlorfructose, 3,6-anhydrogalactose, 1-O-methylgalactose, 1-O-methyl-D-glucose, 1-O-methyl-D-fructose, 3-O-methyl-D-fructose, 6-O-methyl-D-galactose, sedoheptulose, mannoheptulose, L-glycero-D-manno-heptose, and combinations thereof.

Preferably, the sugar alcohol is selected from the group consisting of erythritol, threitol, arabitol, xylitol, ribitol, mannitol, sorbitol, galactitol, fucitol, iditol, inositol, volemitol, isomalt, maltitol, lactitol, and combinations thereof.

Preferably, the sugar acid is selected from the group consisting of xylonic acid, gluconic acid, ascorbic acid, neuraminic acid, ketodeoxyoctonic acid, glucuronic acid, galacturonic acid, iduronic acid, mucic acid, saccharic acid, and combinations thereof.

In one embodiment, the organic polyol is selected from the group consisting of glycerol, sorbitol, xylitol, mannitol, erythritol, maltitol, glucose, glucitol, ribulose, pentaerythritol, and trimethylolpropane. Preferably, the organic polyol is erythritol.

In one embodiment, y is from 2 to the number of hydroxyl groups of the initial organic polyol A. In one embodiment, y is from 3 to the number of hydroxyl groups of the initial organic polyol A. In one embodiment, y is from 4 to the number of hydroxyl groups of the initial organic polyol A. Accordingly, depending on the number of hydroxyl groups of the initial organic polyol A, y can be 1, 2, 3, 4, 5, 6, 7, 8, 9, or 10. In one embodiment, y is 2. In one embodiment, y is 3. In one embodiment, y is 4. In one embodiment, y is 5. In one embodiment, y is 6. In one embodiment, y is 7. In one embodiment, y is 8. In one embodiment, y is 9. In one embodiment, y is 10.

In one embodiment, in the compound according to formula 9, y is equal to the number of hydroxyl groups of the initial polyol A.

In one embodiment, the residues

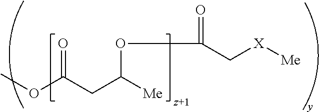

in the compound according to formula 9 may be identical or each independently different for each occurrence.

In one embodiment, the compound according to formula 9, all β-hydroxyl butyric acid ester units are either D-configured or L-configured. In another embodiment, all β-hydroxyl butyric acid ester units are present in the compound according to formula 9 as a non-racemic mixture of D- and L-configurations.

In one embodiment, the compound according to formula 9 contains more D-configured β-hydroxyl butyric acid ester units than L-configured β-hydroxyl butyric acid ester units. Preferably all β-hydroxyl butyric acid ester units are in D-configuration.

In one embodiment, in the compound according to formula 9, all β-hydroxyl butyric acid ester units are either R-configured or S-configured. In another embodiment, all β-hydroxyl butyric acid ester units are present in the compound according to formula 9 as a non-racemic mixture of R- and S-configurations.

In one embodiment, the compound according to formula 9 contains more R-configured β-hydroxyl butyric acid ester units than S-configured β-hydroxyl butyric acid ester units. Preferably all β-hydroxyl butyric acid ester units are in R-configuration.

In one embodiment, X is —C(O)—. In one embodiment, X is —C(H)(OH)—.

In one embodiment, the compound according to formula 9 is selected from the group consisting of

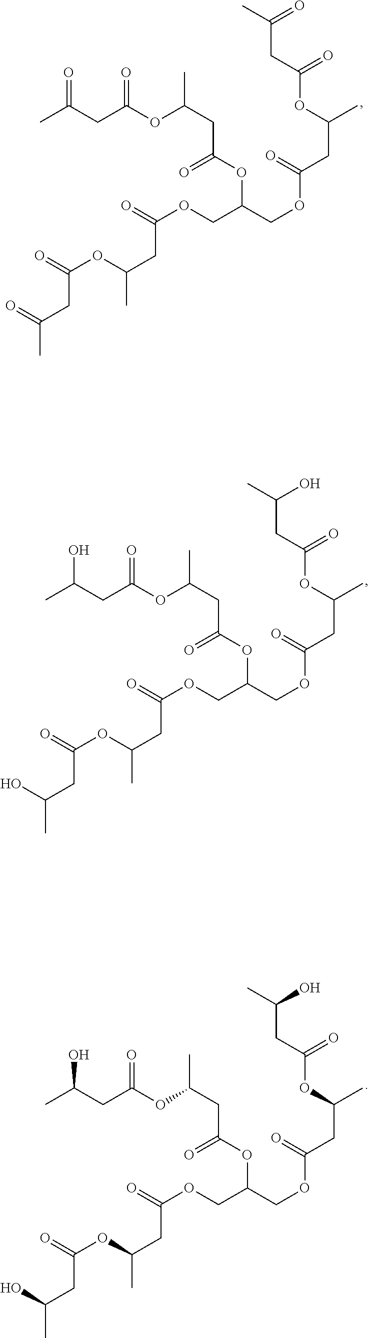 [SMILES: CC(=O)CC(=O)OC(C)CC(=O)OCC(COC(=O)CC(C)OC(=O)CC(C)=O)OC(=O)CC(C)OC(=O)CC(C)=O ; CC(O)CC(=O)OC(C)CC(=O)OCC(COC(=O)CC(C)OC(=O)CC(C)O)OC(=O)CC(C)OC(=O)CC(C)O ; C[C@H](CC(=O)OCC(COC(=O)C[C@H](C)OC(=O)C[C@@H](C)O)OC(=O)C[C@@H](C)OC(=O)C[C@@H](C)O)OC(=O)C[C@@H](C)O]

In another aspect, the present invention provides a compound of formula 9

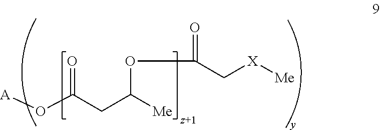

wherein
z is 0 or more,
A is derived from an organic polyol with at least 3 hydroxyl groups, provided that the organic polyol is not erythritol,
X is —C(O)—, —C(H)(OH)—, or —C(H)(OR¹)—,
R¹ is selected from linear or branched C₁₋₁₂ alkyl, C₃₋₈ cycloalkyl, linear or branched C₂₋₁₂ hydroxyalkyl, linear or branched C₁₋₁₂ carboxyalkly, linear or branched and saturated or unsaturated C₁₋₂₄ alkanoyl, phenyl, and carboxyphenyl, and
y is from 1 to the number of hydroxyl groups of the initial polyol A.

In one embodiment, the present invention provides a compound of formula 9

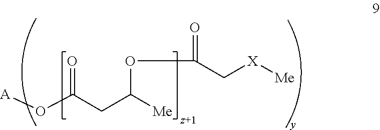

wherein
z is 0 or more,
A is derived from an organic polyol with at least 3 hydroxyl groups, provided that the organic polyol is not erythritol,
X is —C(O)—, —C(H)(OH)—, or —C(H)(OR¹)—,
R¹ is selected from linear or branched C₁₋₁₂ alkyl, C₃₋₈ cycloalkyl, linear or branched C₂₋₁₂ hydroxyalkyl, linear or branched C₁₋₁₂ carboxyalkly, linear or branched and saturated or unsaturated C₁₋₂₄ alkanoyl, phenyl, and carboxyphenyl, and
y is from 3 to the number of hydroxyl groups of the initial polyol A.

In one embodiment, z is from 0-100 such as from 0-95, 0-90, 0-85, 0-80, 0-75, 0-70, 0-65, 0-60, 0-55, 0-50, 0-45, 0-40, 0-35, 0-30, 0-25, or 0-20. In one embodiment, z is from 0-20 such as 0, 1, 2, 3, 4, 5, 6, 7, 8, 9, 10, 11, 12, 13, 14, 15, 16, 17, 18, 19, or 20. In one embodiment, z is from 0-20, such as 0-19, 0-18, 0-17, 0-16, 0-15, 0-14, 0-13, 0-12, 0-11, 0-10, 0-9, 0-8, 0-7, 0-6, 0-5, 0-4, 0-3, 0-2, 1, or 0. Preferably, z is 0 or 1.

In one embodiment, the organic polyol is a linear, branched, or cyclic organic compound with 2 to 18 carbon atoms having at least three hydroxyl groups.

In one embodiment, the organic polyol is selected from a linear or branched C₂₋₁₂ alkyl substituted with at least 3 hydroxyl groups or a C₃₋₈ cycloalkyl substituted with at least 3 hydroxyl groups.

Preferably, the linear or branched C₂₋₁₂ alkyl substituted with at least 3 hydroxyl groups is selected from the group consisting of glycerol, trimethylolpropane, butanetriol, 2-methyl-propanetriol, pentanetriol, 3-methyl-pentanetriol, hexanetriol, pentaerythritol, butanetetrol, pentanetetrol, hexanetetrol, hexanepentol, and combinations thereof.

In one embodiment, the C₃₋₈ cycloalkyl substituted with at least 3 hydroxyl groups is selected from the group consisting of cyclopentanetriol, cyclohexanetriol, cyclopentanetetrol, cyclohexanetetrol, and combinations thereof.

In one embodiment, the organic polyol is selected from the group consisting of monosaccharides, sugar alcohols, and sugar acids.

In one embodiment, the monosaccharide is selected from tetroses, pentoses, hexoses, and heptoses preferably wherein the monosaccharide is selected from aldotetroses, ketotetroses, aldopentoses, ketopentoses, aldohexosen, ketohexoses, aldoheptoses and ketoheptoses.

Preferably, the monosaccharide is selected from the group consisting of erythrose, threose, erythrulose, ribose, arabinose, xylose, lyxose, desoxyribose, ketopentose, ribulose, xylulose, allose, altrose, glucose, mannose, gulose, idose, galactose, talose, n-acetyl-d-glucosamin, glucosamin, N-acetyl-D-galactosamin, fucose, rhamnose, chinovose, fructose, 2-desoxy-D-glucose, fluordesoxyglucose, 6-desoxyfructose, 1,6-dichlorfructose, 3,6-anhydrogalactose, 1-O-methylgalactose, 1-O-methyl-D-glucose, 1-O-methyl-D-fructose, 3-O-methyl-D-fructose, 6-O-methyl-D-galactose, sedoheptulose, mannoheptulose, L-glycero-D-manno-heptose, and combinations thereof.

Preferably, the sugar alcohol is selected from the group consisting of threitol, arabitol, xylitol, ribitol, mannitol, sorbitol, galactitol, fucitol, iditol, inositol, volemitol, isomalt, maltitol, lactitol, and combinations thereof.

Preferably, the sugar acid is selected from the group consisting of xylonic acid, gluconic acid, ascorbic acid, neuraminic acid, ketodeoxyoctonic acid, glucuronic acid, galacturonic acid, iduronic acid, mucic acid, saccharic acid, and combinations thereof.

In one embodiment, the organic polyol is selected from the group consisting of glycerol, sorbitol, xylitol, mannitol, maltitol, glucose, glucitol, ribulose, pentaerythritol, and trimethylolpropane.

In one embodiment, y is from 2 to the number of hydroxyl groups of the initial organic polyol A. In one embodiment, y is from 3 to the number of hydroxyl groups of the initial organic polyol A. In one embodiment, y is from 4 to the number of hydroxyl groups of the initial organic polyol A. Accordingly, depending on the number of hydroxyl groups of the initial organic polyol A, y can be 1, 2, 3, 4, 5, 6, 7, 8, 9, or 10. In one embodiment, y is 2. In one embodiment, y is 3. In one embodiment, y is 4. In one embodiment, y is 5. In one embodiment, y is 6. In one embodiment, y is 7. In one embodiment, y is 8. In one embodiment, y is 9. In one embodiment, y is 10.

In one embodiment, in the compound according to formula 9, y is equal to the number of hydroxyl groups of the initial polyol A.

In one embodiment, the residues

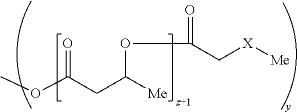

in the compound according to formula 9 may be identical or each independently different for each occurrence.

In one embodiment, the compound according to formula 9, all β-hydroxyl butyric acid ester units are either D-configured or L-configured. In another embodiment, all β-hydroxyl butyric acid ester units are present in the compound according to formula 9 as a non-racemic mixture of D- and L-configurations.

In one embodiment, the compound according to formula 9 contains more D-configured β-hydroxyl butyric acid ester units than L-configured β-hydroxyl butyric acid ester units. Preferably all β-hydroxyl butyric acid ester units are in D-configuration.

In one embodiment, in the compound according to formula 9, all β-hydroxyl butyric acid ester units are either R-configured or S-configured. In another embodiment, all β-hydroxyl butyric acid ester units are present in the compound according to formula 9 as a non-racemic mixture of R- and S-configurations.

In one embodiment, the compound according to formula 9 contains more R-configured β-hydroxyl butyric acid ester units than S-configured β-hydroxyl butyric acid ester units. Preferably all β-hydroxyl butyric acid ester units are in R-configuration.

In one embodiment, X is —C(O)—. In one embodiment, X is —C(H)(OH)—.

In one embodiment, the compound according to formula 9 is selected from the group consisting of

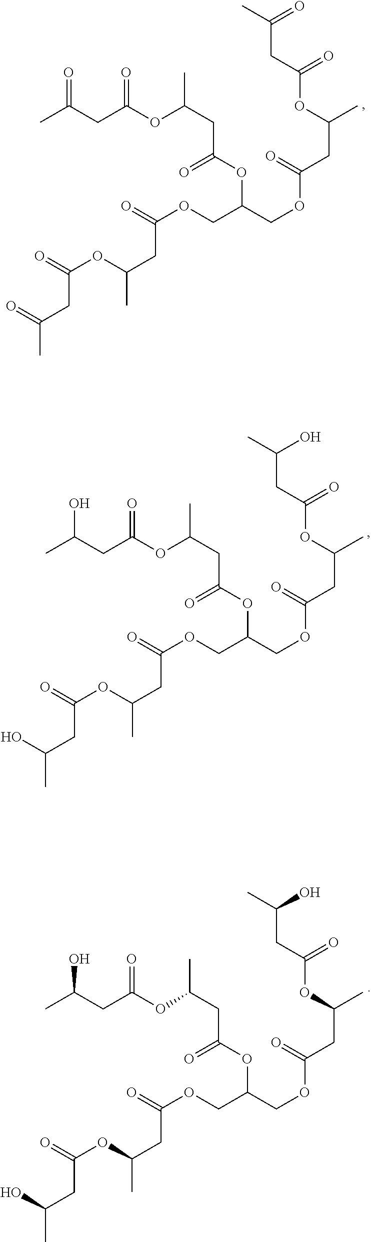 [SMILES: CC(=O)CC(=O)OC(C)CC(=O)OCC(COC(=O)CC(C)OC(=O)CC(C)=O)OC(=O)CC(C)OC(=O)CC(C)=O ; CC(O)CC(=O)OC(C)CC(=O)OCC(COC(=O)CC(C)OC(=O)CC(C)O)OC(=O)CC(C)OC(=O)CC(C)O ; C[C@H](CC(=O)OCC(COC(=O)C[C@H](C)OC(=O)C[C@@H](C)O)OC(=O)C[C@@H](C)OC(=O)C[C@@H](C)O)OC(=O)C[C@@H](C)O]

Preferred embodiments of the present invention are further defined in the following numbered items:
1. A compound of formula 1

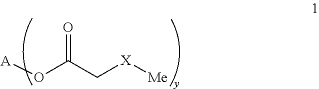

wherein
A is derived from an organic polyol with at least 4 hydroxyl groups,
X is —C(H)(OH)— or —C(H)(OR¹)—,
R¹ is selected from linear or branched C₁₋₁₂ alkyl, C₃₋₈ cycloalkyl, linear or branched C₂₋₁₂ hydroxyalkyl, linear or branched C₁₋₁₂ carboxyalkly, linear or branched and saturated or unsaturated C₁₋₂₄ alkanoyl, phenyl, and carboxyphenyl, and
y is from 1 to the number of hydroxyl groups of the initial organic polyol A.
2. The compound according to item 1, wherein the organic polyol is selected from a linear or branched C₂₋₁₂ alkyl substituted with at least 4 hydroxyl groups or a C₃₋₈ cycloalkyl substituted with at least 4 hydroxyl groups.
3. The compound according to item 2, wherein the linear or branched C₂₋₁₂ alkyl substituted with at least 4 hydroxyl groups is selected from the group consisting of pentaerythritol, butanetetrol, pentanetetrol, hexanetetrol, and hexanepentol.
4. The compound according to item 2 or 3, wherein the C₃₋₈ cycloalkyl substituted with at least 4 hydroxyl groups is selected from the group consisting of cyclopentanetetrol, cyclopentanepentol, cyclohexanetetrol, cyclohexanepentol, and cyclohexanehexol.
5. The compound according to item 1, wherein the organic polyol is selected from the group consisting of monosaccharides, sugar alcohols, and sugar acids.
6. The compound according to item 5, wherein the monosaccharide is selected from pentoses, hexoses, and heptoses, preferably wherein the monosaccharide is selected from aldopentoses, ketopentoses, aldohexosen, ketohexoses, aldoheptoses, and ketoheptoses.
7. The compound according to item 5 or 6, wherein the monosaccharide is selected from the group consisting of ribose, arabinose, xylose, lyxose, ketopentose, ribulose, xylulose, allose, altrose, glucose, mannose, gulose, idose, galactose, talose, n-acetyl-d-glucosamin, glucosamin, N-acetyl-D-galactosamin, fucose, rhamnose, chinovose, fructose, 2-desoxy-D-glucose, fluordesoxyglucose, 6-desoxyfructose, 1,6-dichlorfructose, 3,6-anhydrogalactose, 1-O-methylgalactose, 1-O-methyl-D-glucose, 1-O-methyl-D-fructose, 3-O-methyl-D-fructose, 6-O-methyl-D-galactose, sedoheptulose, mannoheptulose, L-glycero-D-manno-heptose, and combinations thereof.
8. The compound according to any one of items 5 to 7, wherein the sugar alcohol is selected from the group consisting of erythritol, threitol, arabitol, xylitol, ribitol, mannitol, sorbitol, galactitol, fucitol, iditol, inositol, volemitol, isomalt, maltitol, lactitol, and combinations thereof.
9. The compound according to any one of items 5 to 8, wherein the sugar acid is selected from the group consisting of xylonic acid, gluconic acid, ascorbic acid, neuraminic acid, ketodeoxyoctonic acid, glucuronic acid, galacturonic acid, iduronic acid, mucic acid, saccharic acid, and combinations thereof.
10. The compound according to any one of items 1 to 9, wherein the organic polyol is selected from the group consisting of sorbitol, xylitol, mannitol, erythritol, maltitol, glucose, glucitol, ribulose, and pentaerythritol, preferably wherein the organic polyol is erythritol.
11. The compound according to any one of items 1 to 10, wherein the compound is selected from the group consisting of

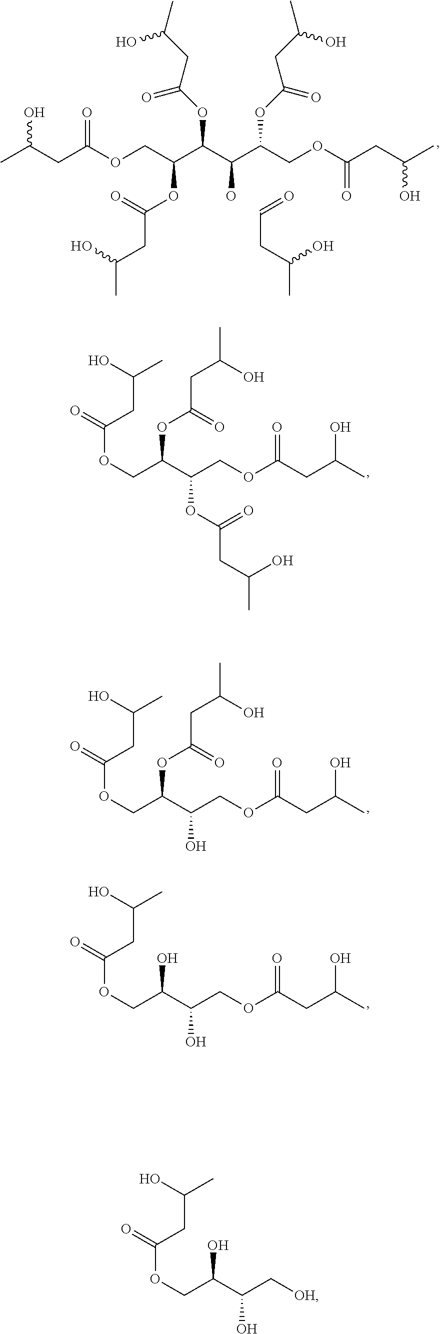

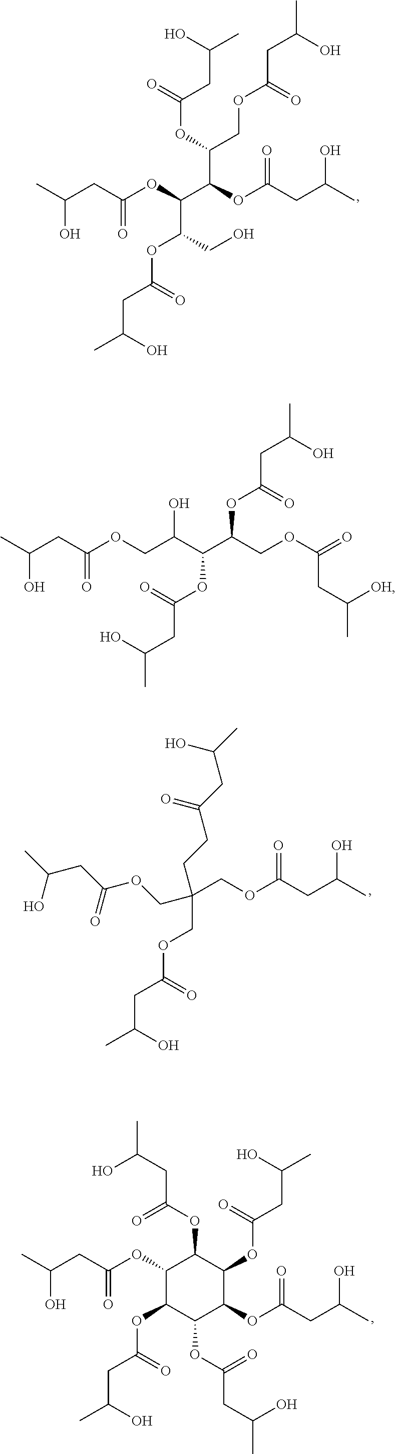

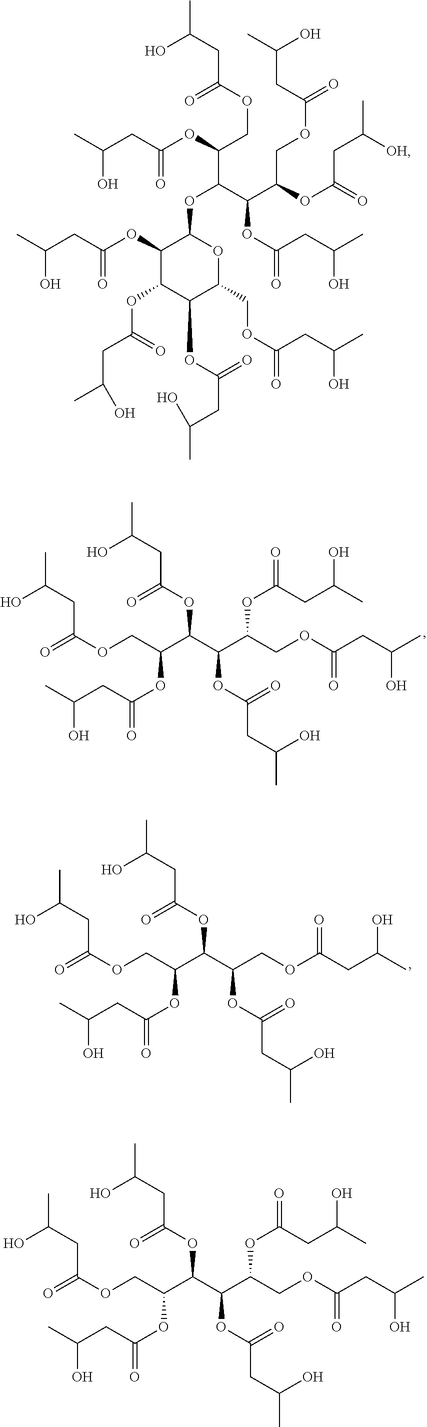 [SMILES: CC(O)CC(=O)OC[C@H](OC(=O)CC(C)O)C(O[C@H]1O[C@H](COC(=O)CC(C)O)[C@@H](OC(=O)CC(C)O)[C@H](OC(=O)CC(C)O)[C@H]1OC(=O)CC(C)O)[C@H](OC(=O)CC(C)O)[C@@H](COC(=O)CC(C)O)OC(=O)CC(C)O ; CC(O)CC(=O)OC[C@H](OC(=O)CC(C)O)[C@@H](OC(=O)CC(C)O)[C@H](OC(=O)CC(C)O)[C@@H](COC(=O)CC(C)O)OC(=O)CC(C)O ; CC(O)CC(=O)OC[C@H](OC(=O)CC(C)O)[C@@H](OC(=O)CC(C)O)[C@@H](COC(=O)CC(C)O)OC(=O)CC(C)O ; CC(O)CC(=O)OC[C@@H](OC(=O)CC(C)O)[C@@H](OC(=O)CC(C)O)[C@H](OC(=O)CC(C)O)[C@@H](COC(=O)CC(C)O)OC(=O)CC(C)O]

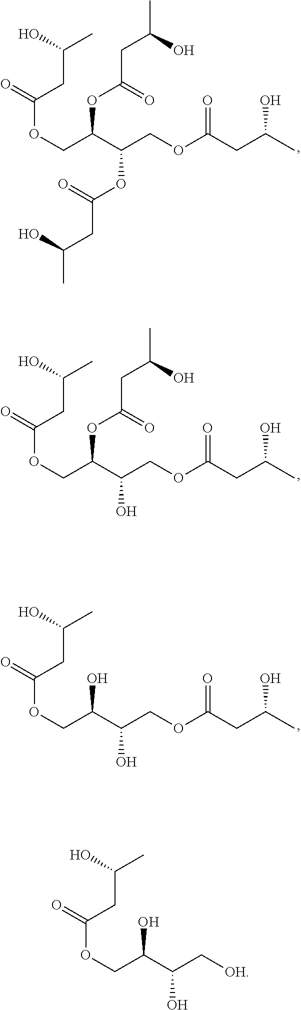 [SMILES: C[C@@H](O)CC(=O)OC[C@H](OC(=O)C[C@@H](C)O)[C@@H](COC(=O)C[C@@H](C)O)OC(=O)C[C@@H](C)O ; C[C@@H](O)CC(=O)OC[C@H](O)[C@@H](COC(=O)C[C@@H](C)O)OC(=O)C[C@@H](C)O ; C[C@@H](O)CC(=O)OC[C@@H](O)[C@@H](O)COC(=O)C[C@@H](C)O ; C[C@@H](O)CC(=O)OC[C@@H](O)[C@@H](O)CO]

12. The compound according to any one of items 1 to 11, wherein all β-hydroxyl butyric acid ester units are either D-configured or L-configured, or all β-hydroxyl butyric acid ester units are present as a non-racemic mixture of D- and L-configurations.
13. The compound according to any one of items 1 to 12, wherein the compound contains more D-configured β-hydroxyl butyric acid ester units than L-configured β-hydroxyl butyric acid ester units, preferably wherein all β-hydroxyl butyric acid ester units are in D-configuration.
14. A compound of formula 9

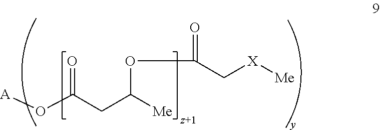

wherein
z is 0 or more,
A is derived from an organic polyol with at least 3 hydroxyl groups,
X is —C(O)—, —C(H)(OH)—, or —C(H)(OR¹)—,
R¹ is selected from linear or branched C₁₋₁₂ alkyl, C₃₋₈ cycloalkyl, linear or branched C₂₋₁₂ hydroxyalkyl, linear or branched C₁₋₁₂ carboxyalkly, linear or branched and saturated or unsaturated C₁₋₂₄ alkanoyl, phenyl, and carboxyphenyl, and
y is from 1 to the number of hydroxyl groups of the initial polyol A.
15. The compound according to item 14, wherein z is from 0-100 such as from 0-95, 0-90, 0-85, 0-80, 0-75, 0-70, 0-65, 0-60, 0-55, 0-50, 0-45, 0-40, 0-35, 0-30, 0-25, or 0-20, preferably z is from 0-20, such as 0-19, such as 0-18, such as 0-17, such as 0-16, such as 0-15, such as 0-14, such as 0-13, such as 0-12, such as 0-11, such as 0-10, such as 0-9, such as 0-8, such as 0-7, such as 0-6, such as 0-5, such as 0-4, such as 0-3, such as 0-2, more preferably wherein z is 0 or 1.
16. The compound according to item 14 or 15, wherein the organic polyol is selected from a linear or branched C₂₋₁₂ alkyl substituted with at least 3 hydroxyl groups or a C₃₋₈ cycloalkyl substituted with at least 3 hydroxyl groups.
17. The compound according to item 16, wherein the linear or branched C₂₋₁₂ alkyl substituted with at least 3 hydroxyl groups is selected from the group consisting of glycerol, trimethylolpropane, butanetriol, 2-methyl-propanetriol, pentanetriol, 3-methyl-pentanetriol, hexanetriol, pentaerythritol, butanetetrol, pentanetetrol, hexanetetrol, hexanepentol, and combinations thereof.
18. The compound according to item 16 or 17, wherein the C₃₋₈ cycloalkyl substituted with at least 3 hydroxyl groups is selected from the group consisting of cyclopentanetriol, cyclohexanetriol, cyclopentanetetrol, cyclohexanetetrol, and combinations thereof.
19. The compound according to item 14 or 15, wherein the organic polyol is selected from the group consisting of monosaccharides, sugar alcohols, and sugar acids.
20. The compound according to item 19, wherein the monosaccharide is selected from tetroses, pentoses, hexoses, and heptoses preferably wherein the monosaccharide is selected from aldotetroses, ketotetroses, aldopentoses, ketopentoses, aldohexosen, ketohexoses, aldoheptoses and ketoheptoses.
21. The compound according to item 19 or 20, wherein the monosaccharide is selected from the group consisting of erythrose, threose, erythrulose, ribose, arabinose, xylose, lyxose, desoxyribose, ketopentose, ribulose, xylulose, allose, altrose, glucose, mannose, gulose, idose, galactose, talose, n-acetyl-d-glucosamin, glucosamin, N-acetyl-D-galactosamin, fucose, rhamnose, chinovose, fructose, 2-desoxy-D-glucose, fluordesoxyglucose, 6-desoxyfructose, 1,6-dichlorfructose, 3,6-anhydrogalactose, 1-O-methylgalactose, 1-O-methyl-D-glucose, 1-O-methyl-D-fructose, 3-O-methyl-D-fructose, 6-O-methyl-D-galactose, sedoheptulose, mannoheptulose, L-glycero-D-manno-heptose, and combinations thereof.
22. The compound according to any one of items 19 to 21, wherein the sugar alcohol is selected from the group consisting of erythritol, threitol, arabitol, xylitol, ribitol, mannitol, sorbitol, galactitol, fucitol, iditol, inositol, volemitol, isomalt, maltitol, lactitol, and combinations thereof.
23. The compound according to any one of items 19 to 22, wherein the sugar acid is selected from the group consisting of xylonic acid, gluconic acid, ascorbic acid, neuraminic acid, ketodeoxyoctonic acid, glucuronic acid, galacturonic acid, iduronic acid, mucic acid, saccharic acid, and combinations thereof.
24. The compound according to any one of items 14 to 23, wherein the organic polyol is selected from the group consisting of glycerol, sorbitol, xylitol, mannitol, erythritol, maltitol, glucose, glucitol, ribulose, pentaerythritol, and trimethylolpropane, preferably wherein the organic polyol is erythritol.
25. The compound according to any one of items 14 to 24, wherein the compound is selected from the group consisting of

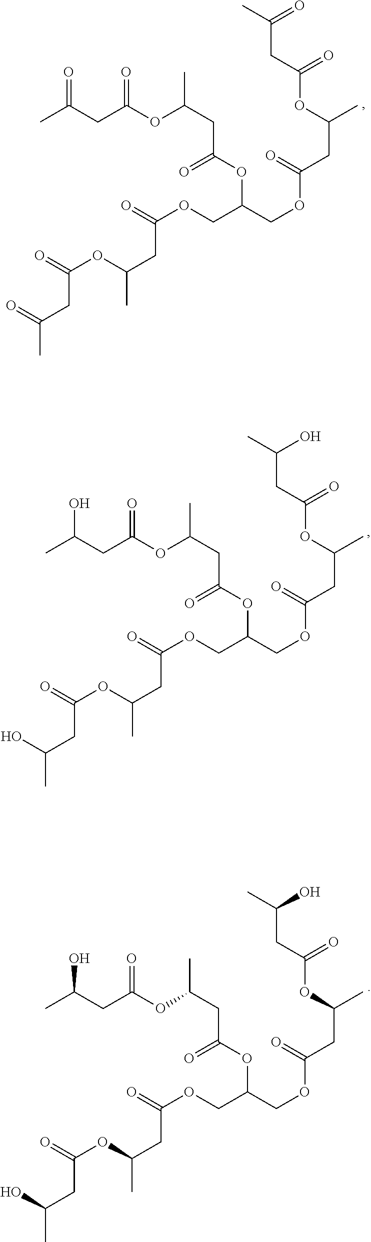 [SMILES: CC(=O)CC(=O)OC(C)CC(=O)OCC(COC(=O)CC(C)OC(=O)CC(C)=O)OC(=O)CC(C)OC(=O)CC(C)=O ; CC(O)CC(=O)OC(C)CC(=O)OCC(COC(=O)CC(C)OC(=O)CC(C)O)OC(=O)CC(C)OC(=O)CC(C)O ; C[C@H](CC(=O)OCC(COC(=O)C[C@H](C)OC(=O)C[C@@H](C)O)OC(=O)C[C@@H](C)OC(=O)C[C@@H](C)O)OC(=O)C[C@@H](C)O]

26. The compound according to any one of items 14 to 25, wherein all β-hydroxyl butyric acid ester units are either D-configured or L-configured, or all β-hydroxyl butyric acid ester units are present as a non-racemic mixture of D- and L-configurations.
27. The compound according to any one of items 14 to 26, wherein the compound contains more D-configured β-hydroxyl butyric acid ester units than L-configured β-hydroxyl butyric acid ester units, preferably wherein all β-hydroxyl butyric acid ester units are in D-configuration.
28. The compound according to any one of items 1 to 27, wherein y is from 3 to the number of hydroxyl groups of the initial polyol A, y is from 4 to the number of hydroxyl groups of the initial polyol A, or y is equal to the number of hydroxyl groups of the initial polyol A.
29. A process for the preparation of a compound of formula 1

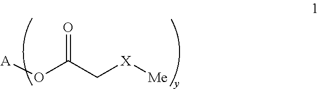

wherein
A is derived from an organic polyol with at least 4 hydroxyl groups,
X is —C(H)(OH)— or —C(H)(OR¹)—,
R¹ is selected from linear or branched C₁₋₁₂ alkyl, C₃₋₈ cycloalkyl, linear or branched C₂₋₁₂ hydroxyalkyl, linear or branched C₁₋₁₂ carboxyalkly, linear or branched and saturated or unsaturated C₁₋₂₄ alkanoyl, phenyl, and carboxyphenyl, and
y is from 1 to the number of hydroxyl groups of the initial organic polyol A;
wherein the process comprises:
   (i) reacting an organic polyol of formula 2 with diketene 3 resulting in the formation of a compound according to formula 4;

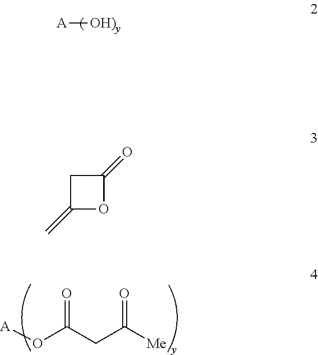

and
(iia) reacting the compound of formula 4 with hydrogen in the presence of a catalyst resulting in the formation of a compound according to formula 5,

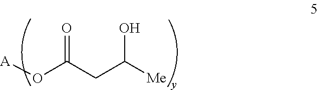

and optionally
(iib) reacting the compound of formula 5 with a compound LG-R¹, wherein LG is a leaving group, resulting in the formation of a compound according to formula 6;

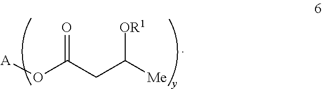

30. The process according to item 29, wherein the organic polyol is selected from a linear or branched C₂₋₁₂ alkyl substituted with at least 4 hydroxyl groups or a C₃₋₈ cycloalkyl substituted with at least 4 hydroxyl groups.
31. The process according to item 30, wherein the linear or branched C₂₋₁₂ alkyl substituted with at least 4 hydroxyl groups is selected from the group consisting of pentaerythritol, butanetetrol, pentanetetrol, hexanetetrol, and hexanepentol.
32. The process according to item 30 or 31, wherein the C₃₋₈ cycloalkyl substituted with at least 4 hydroxyl groups is selected from the group consisting of cyclopentanetetrol, cyclopentanepentol, cyclohexanetetrol, cyclohexanepentol, and cyclohexanehexole.
33. The process according to item 29, wherein the organic polyol is selected from the group consisting of monosaccharides, sugar alcohols, and sugar acids.
34. The process according to item 33, wherein the monosaccharide is selected from pentoses, hexoses, and heptoses, preferably wherein the monosaccharide is selected from aldopentoses, ketopentoses, aldohexosen, ketohexoses, aldoheptoses, and ketoheptoses.
35. The process according to item 33 or 34, wherein the monosaccharide is selected from the group consisting of ribose, arabinose, xylose, lyxose, ketopentose, ribulose, xylulose, allose, altrose, glucose, mannose, gulose, idose, galactose, talose, n-acetyl-d-glucosamin, glucosamin, N-acetyl-D-galactosamin, fucose, rhamnose, chinovose, fructose, 2-desoxy-D-glucose, fluordesoxyglucose, 6-desoxyfructose, 1,6-dichlorfructose, 3,6-anhydrogalactose, 1-O-methylgalactose, 1-O-methyl-D-glucose, 1-O-methyl-D-fructose, 3-O-methyl-D-fructose, 6-O-methyl-D-galactose, sedoheptulose, mannoheptulose, L-glycero-D-manno-heptose, and combinations thereof.
36. The process according to any one of items 34 to 35, wherein the sugar alcohol is selected from the group consisting of erythritol, threitol, arabitol, xylitol, ribitol, mannitol, sorbitol, galactitol, fucitol, iditol, inositol, volemitol, isomalt, maltitol, lactitol, and combinations thereof.
37. The process according to any one of items 33 to 36, wherein the sugar acid is selected from the group consisting of xylonic acid, gluconic acid, ascorbic acid, neuraminic acid, ketodeoxyoctonic acid, glucuronic acid, galacturonic acid, iduronic acid, mucic acid, saccharic acid, and combinations thereof.
38. The process according to any one of items 29 to 37, wherein the organic polyol is selected from the group consisting of sorbitol, xylitol, mannitol, erythritol, maltitol, glucose, glucitol, ribulose, and pentaerythritol, preferably wherein the organic polyol is erythritol.
39. The process according to any one of items 29 to 38, wherein the compound is selected from the group consisting of

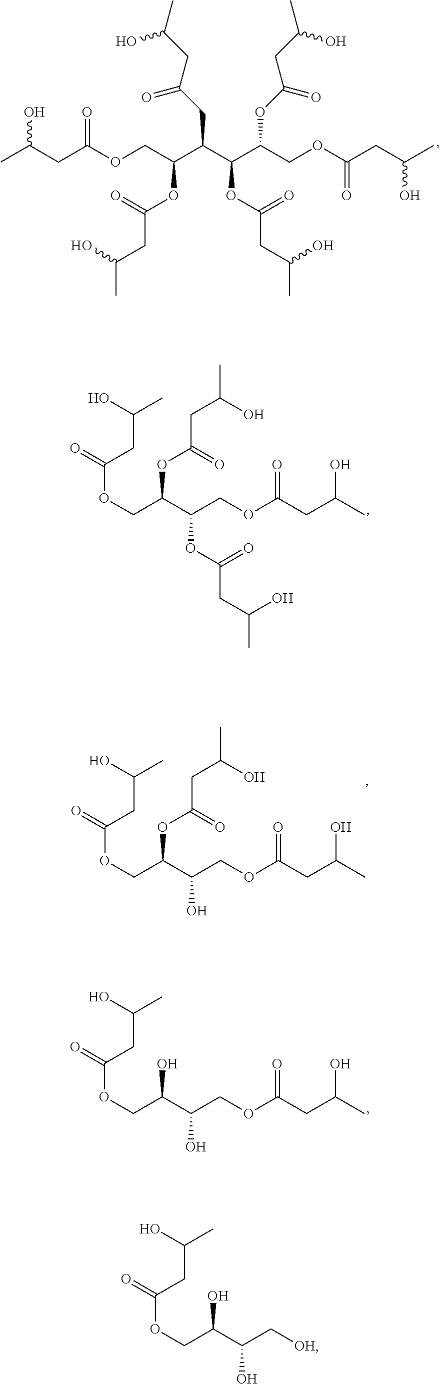 [SMILES: CC(O)CC(=O)C[C@@H]([C@H](OC(=O)CC(C)O)[C@@H](COC(=O)CC(C)O)OC(=O)CC(C)O)[C@H](COC(=O)CC(C)O)OC(=O)CC(C)O ; CC(O)CC(=O)OC[C@H](OC(=O)CC(C)O)[C@@H](COC(=O)CC(C)O)OC(=O)CC(C)O ; CC(O)CC(=O)OC[C@H](O)[C@@H](COC(=O)CC(C)O)OC(=O)CC(C)O ; CC(O)CC(=O)OC[C@@H](O)[C@@H](O)COC(=O)CC(C)O ; CC(O)CC(=O)OC[C@@H](O)[C@@H](O)CO]

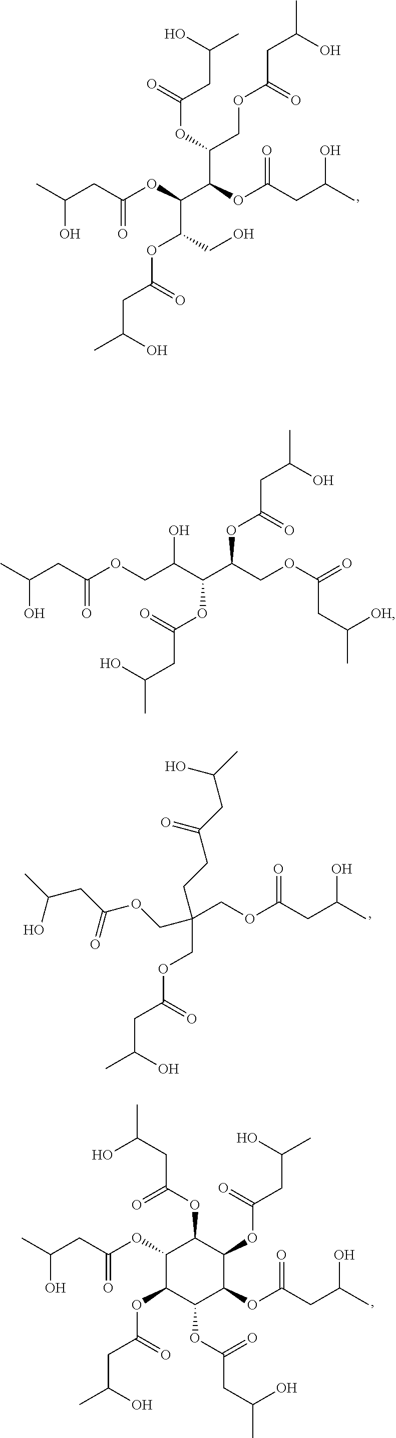

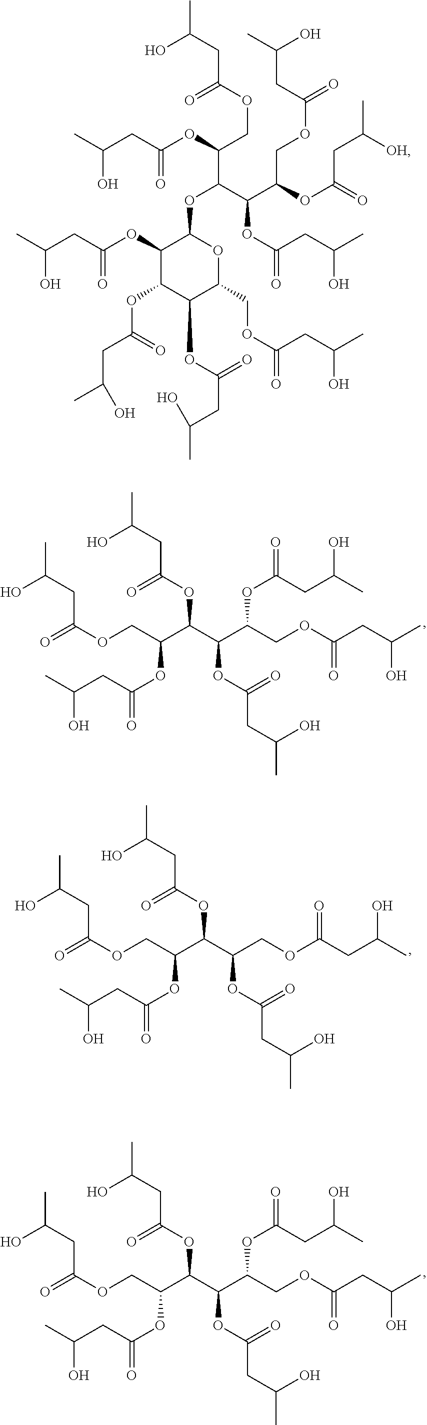 [SMILES: CC(O)CC(=O)OC[C@H](OC(=O)CC(C)O)C(O[C@H]1O[C@H](COC(=O)CC(C)O)[C@@H](OC(=O)CC(C)O)[C@H](OC(=O)CC(C)O)[C@H]1OC(=O)CC(C)O)[C@H](OC(=O)CC(C)O)[C@@H](COC(=O)CC(C)O)OC(=O)CC(C)O ; CC(O)CC(=O)OC[C@H](OC(=O)CC(C)O)[C@@H](OC(=O)CC(C)O)[C@H](OC(=O)CC(C)O)[C@@H](COC(=O)CC(C)O)OC(=O)CC(C)O ; CC(O)CC(=O)OC[C@H](OC(=O)CC(C)O)[C@@H](OC(=O)CC(C)O)[C@@H](COC(=O)CC(C)O)OC(=O)CC(C)O ; CC(O)CC(=O)OC[C@@H](OC(=O)CC(C)O)[C@@H](OC(=O)CC(C)O)[C@H](OC(=O)CC(C)O)[C@@H](COC(=O)CC(C)O)OC(=O)CC(C)O]

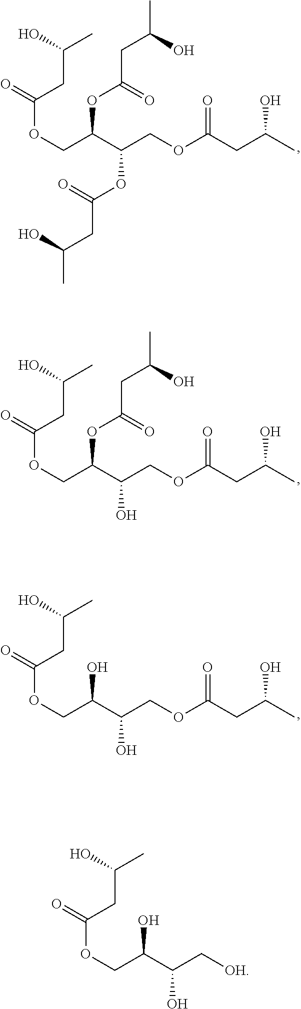 [SMILES: C[C@@H](O)CC(=O)OC[C@H](OC(=O)C[C@@H](C)O)[C@@H](COC(=O)C[C@@H](C)O)OC(=O)C[C@@H](C)O ; C[C@@H](O)CC(=O)OC[C@H](O)[C@@H](COC(=O)C[C@@H](C)O)OC(=O)C[C@@H](C)O ; C[C@@H](O)CC(=O)OC[C@@H](O)[C@@H](O)COC(=O)C[C@@H](C)O ; C[C@@H](O)CC(=O)OC[C@@H](O)[C@@H](O)CO]

40. A process for the preparation of a compound of formula 9

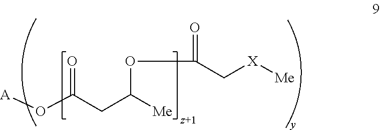

wherein
z is 0 or more,
A is derived from an organic polyol with at least 3 hydroxyl groups,
X is —C(O)—, —C(H)(OH)—, or —C(H)(OR¹)—,
R¹ is selected from linear or branched C₁₋₁₂ alkyl, C₃₋₈ cycloalkyl, linear or branched C₂₋₁₂ hydroxyalkyl, linear or branched C₁₋₁₂ carboxyalkly, linear or branched and saturated or unsaturated C₁₋₂₄ alkanoyl, phenyl, and carboxyphenyl, and
y is from 1 to the number of hydroxyl groups of the initial organic polyol A;
wherein the process comprises:
   (i) reacting a compound of formula 10 with diketene 3 resulting in the formation of a compound according to formula 11;

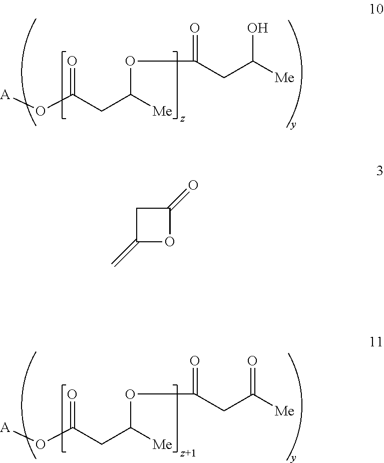

optionally
(iia) reacting the compound of formula 11 with hydrogen in the presence of a catalyst resulting in the formation of a compound according to formula 12,

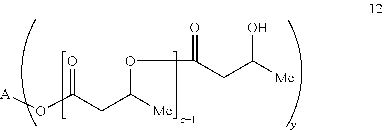

and optionally
(iib) reacting the compound of formula 12 with a compound LG-R¹, wherein LG is a leaving group, resulting in the formation of a compound according to formula 13;

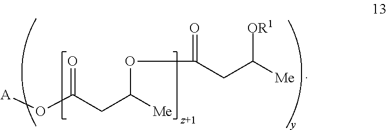

41. The process according to item 40, wherein z is from 0-100 such as from 0-95, 0-90, 0-85, 0-80, 0-75, 0-70, 0-65, 0-60, 0-55, 0-50, 0-45, 0-40, 0-35, 0-30, 0-25, or 0-20, preferably z is from 0-20, such as 0-19, such as 0-18, such as 0-17, such as 0-16, such as 0-15, such as 0-14, such as 0-13, such as 0-12, such as 0-11, such as 0-10, such as 0-9, such as 0-8, such as 0-7, such as 0-6, such as 0-5, such as 0-4, such as 0-3, such as 0-2, more preferably wherein z is 0 or 1.
42. The process according to item 40 or 41, wherein the organic polyol is selected from a linear or branched C₂₋₁₂ alkyl substituted with at least 3 hydroxyl groups or a C₃₋₈ cycloalkyl substituted with at least 3 hydroxyl groups.
43. The process according to item 42, wherein the linear or branched C₂₋₁₂ alkyl substituted with at least 3 hydroxyl groups is selected from the group consisting of glycerol, trimethylolpropane, butanetriol, 2-methyl-propanetriol, pentanetriol, 3-methyl-pentanetriol, hexanetriol, pentaerythritol, butanetetrol, pentanetetrol, hexanetetrol, hexanepentol, and combinations thereof.
44. The process according to item 42 or 43, wherein the C₃₋₈ cycloalkyl substituted with at least 3 hydroxyl groups is selected from the group consisting of cyclopentanetriol, cyclohexanetriol, cyclopentanetetrol, cyclohexanetetrol, and combinations thereof.
45. The process according to item 40 or 41, wherein the organic polyol is selected from the group consisting of monosaccharides, sugar alcohols, and sugar acids.
46. The process according to item 45, wherein the monosaccharide is selected from tetroses, pentoses, hexoses, and heptoses, preferably wherein the monosaccharide is selected from aldotetroses, ketotetroses, aldopentoses, ketopentoses, aldohexosen, ketohexoses, aldoheptoses and ketoheptoses.
47. The process according to item 45 or 46, wherein the monosaccharide is selected from the group consisting of erythrose, threose, erythrulose, ribose, arabinose, xylose, lyxose, desoxyribose, ketopentose, ribulose, xylulose, allose, altrose, glucose, mannose, gulose, idose, galactose, talose, n-acetyl-d-glucosamin, glucosamin, N-acetyl-D-galactosamin, fucose, rhamnose, chinovose, fructose, 2-desoxy-D-glucose, fluordesoxyglucose, 6-desoxyfructose, 1,6-dichlorfructose, 3,6-anhydrogalactose, 1-O-methylgalactose, 1-O-methyl-D-glucose, 1-O-methyl-D-fructose, 3-O-methyl-D-fructose, 6-O-methyl-D-galactose, sedoheptulose, mannoheptulose, L-glycero-D-manno-heptose, and combinations thereof.
48. The process according to any one of items 45 to 47, wherein the sugar alcohol is selected from the group consisting of erythritol, threitol, arabitol, xylitol, ribitol, mannitol, sorbitol, galactitol, fucitol, iditol, inositol, volemitol, isomalt, maltitol, lactitol, and combinations thereof.
49. The process according to any one of items 45 to 48, wherein the sugar acid is selected from the group consisting of xylonic acid, gluconic acid, ascorbic acid, neuraminic acid, ketodeoxyoctonic acid, glucuronic acid, galacturonic acid, iduronic acid, mucic acid, saccharic acid, and combinations thereof.
50. The process according to any one of items 40 to 49, wherein the organic polyol is selected from the group consisting of glycerol, sorbitol, xylitol, mannitol, erythritol, maltitol, glucose, glucitol, ribulose, pentaerythritol, and trimethylolpropane, preferably wherein the organic polyol is erythritol.
51. The process according to any one of items 40 to 50, wherein the compound is selected from the group consisting of

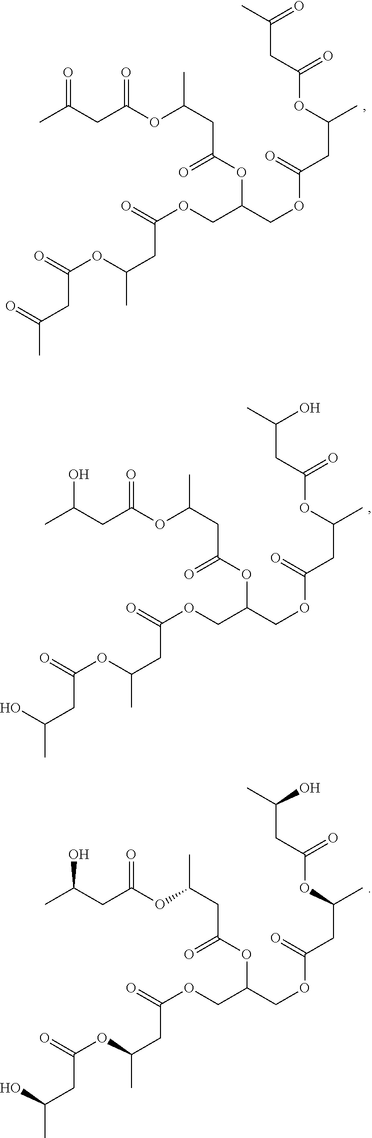 [SMILES: CC(=O)CC(=O)OC(C)CC(=O)OCC(COC(=O)CC(C)OC(=O)CC(C)=O)OC(=O)CC(C)OC(=O)CC(C)=O ; CC(O)CC(=O)OC(C)CC(=O)OCC(COC(=O)CC(C)OC(=O)CC(C)O)OC(=O)CC(C)OC(=O)CC(C)O ; C[C@H](CC(=O)OCC(COC(=O)C[C@H](C)OC(=O)C[C@@H](C)O)OC(=O)C[C@@H](C)OC(=O)C[C@@H](C)O)OC(=O)C[C@@H](C)O]

52. The process according to any one of items 29 to 51, wherein reaction step (i) is performed in the presence of an organic amine catalyst.
53. The process according to item 52, wherein the organic amine catalyst is a tertiary amine.
54. The process according to item 53, wherein the organic amine catalyst is DABCO.
55. The process according to any one of items 29 to 54, wherein reaction step (iia) is performed in the presence of a metal-based catalyst, preferably a Ni-based catalyst, a Pd-based catalyst, a Pt-based catalyst, a Ru-based catalyst, a Co-based catalyst, an Ir-based catalyst, or a Rh-based catalyst.
56. The process according to any one of items 29 to 55, wherein reaction step (iia) is performed in the presence of a Ru-based catalyst, preferably selected from a Ruthenium oxide catalyst, Ru/C, RuAl₂O₃, RuO₂, Ru(OAc)₂(BINAP), Ru(Cl)₂(BINAP), C3-[(S,S)-teth-MtsDpenRuCl], [(R)-BinapRuCl(p-cymene)]Cl, and [Chloro(R)—C3-TunePhos)(p-cymene)ruthenium(II)]chloride.
57. The process according to any one of items 29 to 56, wherein reaction step (iia) is performed in the presence of a chiral ligand capable of forming complexes with the metal-based catalyst, preferably wherein the chiral ligand is selected from the group consisting of 2,2′-bis(diphenylphosphino)-1,1′-binaphthyl (BINAP), 1,1′-Bi-2-naphthol (BINOL), 2,3-O-isopropylidene-2,3-dihydroxy-1,4-bis(diphenylphosphino)butane (DIOP), 2,2′,5,5′-tetramethyl-4,4′-bis-(diphenylphoshino)-3,3′-bithiophene (tetraMe-BITIOP), Bis(diphenylphosphino)-7,8-dihydro-6H-dibenzo[f,h][1,5]dioxonin (C3-TunePhos), 4,4′-Bis(bis(3,5-dimethylphenyl)phosphino)-2,2′,6,6′-tetramethoxy-3,3′-bipyridine (Xyl-p-PHOS), (6,6′-Dimethoxybiphenyl-2,2′-diyl)-bis-(diphenylphosphin) (MeO-BIPHEP), and 1,2-Bis[(2-methoxyphenyl)phenylphosphino]ethane (DIPAMP).
58. The process according to any one of items 29 to 56, wherein all β-hydroxyl butyric acid ester units are either D-configured or L-configured, or all β-hydroxyl butyric acid ester units are present as a non-racemic mixture of D- and L-configurations.
59. The process according to any one of items 29 to 58, wherein the compound contains more D-configured β-hydroxyl butyric acid ester units than L-configured β-hydroxyl butyric acid ester units, preferably wherein all β-hydroxyl butyric acid ester units are in D-configuration.
60. The process according to any one of items 29 to 59, wherein compounds 5 or 12 are further esterified with an omega fatty acid, a medium-chain fatty acid, or a combination thereof at the hydroxyl group of at least one of the terminal β-hydroxyl butyric acid ester unit, preferably wherein the omega fatty acid is an omega-3 fatty acid, an omega-6 fatty acid, an omega-3,6 fatty acid, or a combination thereof, and/or wherein the medium-chain fatty acid is selected from the group consisting of caproic acid, caprylic acid, capric acid, lauric acid and combinations thereof.
61. The process according to any one of items 29 to 60, wherein y is from 3 to the number of hydroxyl groups of the initial polyol A, y is from 4 to the number of hydroxyl groups of the initial polyol A, or y is equal to the number of hydroxyl groups of the initial polyol A.
62. A compound of formula 9

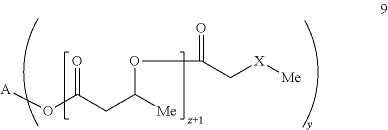

wherein
z is 0 or 1,
A is derived from an organic polyol with at least 3 hydroxyl groups,
X is —C(O)—, —C(H)(OH)—, or —C(H)(OR¹)—,
R¹ is selected from linear or branched C₁₋₁₂ alkyl, C₃₋₈ cycloalkyl, linear or branched C₂₋₁₂ hydroxyalkyl, linear or branched C₁₋₁₂ carboxyalkly, linear or branched and saturated or unsaturated C₁₋₂₄ alkanoyl, phenyl, and carboxyphenyl, and
y is from 1 to the number of hydroxyl groups of the initial polyol A.
63. The compound according to item 62, wherein the organic polyol is selected from a linear or branched C₂₋₁₂ alkyl substituted with at least 3 hydroxyl groups or a C₃₋₈ cycloalkyl substituted with at least 3 hydroxyl groups.
64. The compound according to item 63, wherein the linear or branched C₂₋₁₂ alkyl substituted with at least 3 hydroxyl groups is selected from the group consisting of glycerol, trimethylolpropane, butanetriol, 2-methyl-propanetriol, pentanetriol, 3-methyl-pentanetriol, hexanetriol, pentaerythritol, butanetetrol, pentanetetrol, hexanetetrol, hexanepentol, and combinations thereof.
65. The compound according to item 63 or 64, wherein the C₃₋₈ cycloalkyl substituted with at least 3 hydroxyl groups is selected from the group consisting of cyclopentanetriol, cyclohexanetriol, cyclopentanetetrol, cyclohexanetetrol, and combinations thereof.
66. The compound according to item 62, wherein the organic polyol is selected from the group consisting of monosaccharides, sugar alcohols, and sugar acids.
67. The compound according to item 66, wherein the monosaccharide is selected from tetroses, pentoses, hexoses, and heptoses preferably wherein the monosaccharide is selected from aldotetroses, ketotetroses, aldopentoses, ketopentoses, aldohexosen, ketohexoses, aldoheptoses and ketoheptoses.
68. The compound according to item 66 or 67, wherein the monosaccharide is selected from the group consisting of erythrose, threose, erythrulose, ribose, arabinose, xylose, lyxose, desoxyribose, ketopentose, ribulose, xylulose, allose, altrose, glucose, mannose, gulose, idose, galactose, talose, n-acetyl-d-glucosamin, glucosamin, N-acetyl-D-galactosamin, fucose, rhamnose, chinovose, fructose, 2-desoxy-D-glucose, fluordesoxyglucose, 6-desoxyfructose, 1,6-dichlorfructose, 3,6-anhydrogalactose, 1-O-methylgalactose, 1-O-methyl-D-glucose, 1-O-methyl-D-fructose, 3-O-methyl-D-fructose, 6-O-methyl-D-galactose, sedoheptulose, mannoheptulose, L-glycero-D-manno-heptose, and combinations thereof.
69. The compound according to any one of items 66 to 68, wherein the sugar alcohol is selected from the group consisting of erythritol, threitol, arabitol, xylitol, ribitol, mannitol, sorbitol, galactitol, fucitol, iditol, inositol, volemitol, isomalt, maltitol, lactitol, and combinations thereof.
70. The compound according to any one of items 66 to 69, wherein the sugar acid is selected from the group consisting of xylonic acid, gluconic acid, ascorbic acid, neuraminic acid, ketodeoxyoctonic acid, glucuronic acid, galacturonic acid, iduronic acid, mucic acid, saccharic acid, and combinations thereof.
71. The compound according to any one of items 62 to 70, wherein the organic polyol is selected from the group consisting of glycerol, sorbitol, xylitol, mannitol, erythritol, maltitol, glucose, glucitol, ribulose, pentaerythritol, and trimethylolpropane, preferably wherein the organic polyol is erythritol.
72. The compound according to any one of items 62 to 71, wherein the compound is selected from the group consisting of

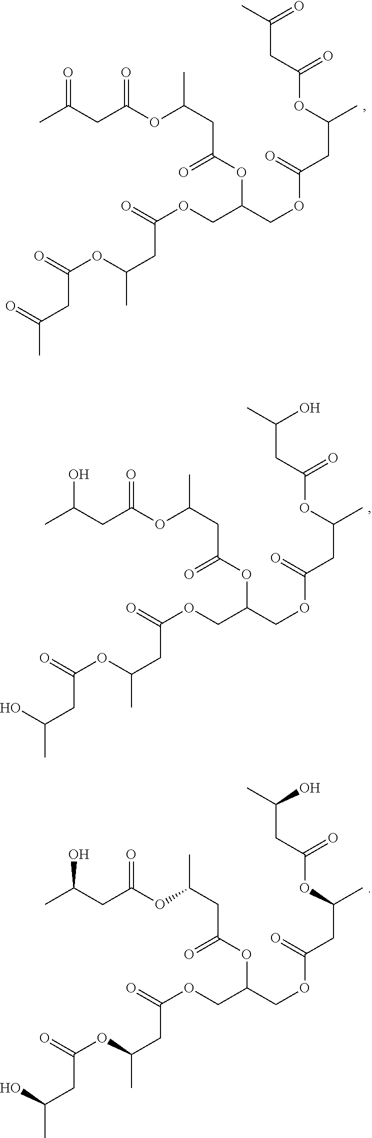 [SMILES: CC(=O)CC(=O)OC(C)CC(=O)OCC(COC(=O)CC(C)OC(=O)CC(C)=O)OC(=O)CC(C)OC(=O)CC(C)=O ; CC(O)CC(=O)OC(C)CC(=O)OCC(COC(=O)CC(C)OC(=O)CC(C)O)OC(=O)CC(C)OC(=O)CC(C)O ; C[C@H](CC(=O)OCC(COC(=O)C[C@H](C)OC(=O)C[C@@H](C)O)OC(=O)C[C@@H](C)OC(=O)C[C@@H](C)O)OC(=O)C[C@@H](C)O]

73. The compound according to any one of items 62 to 72, wherein all β-hydroxyl butyric acid ester units are either D-configured or L-configured, or all β-hydroxyl butyric acid ester units are present as a non-racemic mixture of D- and L-configurations.
74. The compound according to any one of items 62 to 73, wherein the compound contains more D-configured β-hydroxyl butyric acid ester units than L-configured β-hydroxyl butyric acid ester units, preferably wherein all β-hydroxyl butyric acid ester units are in D-configuration.
75. The compound according to any one of items 62 to 74, wherein y is from 3 to the number of hydroxyl groups of the initial polyol A, y is from 4 to the number of hydroxyl groups of the initial polyol A, or y is equal to the number of hydroxyl groups of the initial polyol A.
76. A compound of formula 9

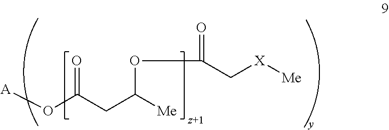

wherein
z is 0 or more,
A is derived from an organic polyol with at least 3 hydroxyl groups, provided that the organic polyol is not erythritol,
X is —C(O)—, —C(H)(OH)—, or —C(H)(OR¹)—,
R¹ is selected from linear or branched C₁₋₁₂ alkyl, C₃₋₈ cycloalkyl, linear or branched C₂₋₁₂ hydroxyalkyl, linear or branched C₁₋₁₂ carboxyalkly, linear or branched and saturated or unsaturated C₁₋₂₄ alkanoyl, phenyl, and carboxyphenyl, and
y is from 1 to the number of hydroxyl groups of the initial polyol A.
77. The compound according to item 76, wherein z is from 0-100 such as from 0-95, 0-90, 0-85, 0-80, 0-75, 0-70, 0-65, 0-60, 0-55, 0-50, 0-45, 0-40, 0-35, 0-30, 0-25, or 0-20, preferably z is from 0-20, such as 0-19, such as 0-18, such as 0-17, such as 0-16, such as 0-15, such as 0-14, such as 0-13, such as 0-12, such as 0-11, such as 0-10, such as 0-9, such as 0-8, such as 0-7, such as 0-6, such as 0-5, such as 0-4, such as 0-3, such as 0-2, more preferably wherein z is 0 or 1.
78. The compound according to item 76 or 77, wherein the organic polyol is selected from a linear or branched C₂₋₁₂ alkyl substituted with at least 3 hydroxyl groups or a C₃₋₈ cycloalkyl substituted with at least 3 hydroxyl groups.
79. The compound according to item 78, wherein the linear or branched C₂₋₁₂ alkyl substituted with at least 3 hydroxyl groups is selected from the group consisting of glycerol, trimethylolpropane, butanetriol, 2-methyl-propanetriol, pentanetriol, 3-methyl-pentanetriol, hexanetriol, pentaerythritol, butanetetrol, pentanetetrol, hexanetetrol, hexanepentol, and combinations thereof.
80. The compound according to item 78 or 79, wherein the C₃₋₈ cycloalkyl substituted with at least 3 hydroxyl groups is selected from the group consisting of cyclopentanetriol, cyclohexanetriol, cyclopentanetetrol, cyclohexanetetrol, and combinations thereof.
81. The compound according to item 76 or 77, wherein the organic polyol is selected from the group consisting of monosaccharides, sugar alcohols, and sugar acids.
82. The compound according to item 81, wherein the monosaccharide is selected from tetroses, pentoses, hexoses, and heptoses preferably wherein the monosaccharide is selected from aldotetroses, ketotetroses, aldopentoses, ketopentoses, aldohexosen, ketohexoses, aldoheptoses and ketoheptoses.
83. The compound according to item 81 or 82, wherein the monosaccharide is selected from the group consisting of erythrose, threose, erythrulose, ribose, arabinose, xylose, lyxose, desoxyribose, ketopentose, ribulose, xylulose, allose, altrose, glucose, mannose, gulose, idose, galactose, talose, n-acetyl-d-glucosamin, glucosamin, N-acetyl-D-galactosamin, fucose, rhamnose, chinovose, fructose, 2-desoxy-D-glucose, fluordesoxyglucose, 6-desoxyfructose, 1,6-dichlorfructose, 3,6-anhydrogalactose, 1-O-methylgalactose, 1-O-methyl-D-glucose, 1-O-methyl-D-fructose, 3-O-methyl-D-fructose, 6-O-methyl-D-galactose, sedoheptulose, mannoheptulose, L-glycero-D-manno-heptose, and combinations thereof.
84. The compound according to any one of items 81 to 83, wherein the sugar alcohol is selected from the group consisting of threitol, arabitol, xylitol, ribitol, mannitol, sorbitol, galactitol, fucitol, iditol, inositol, volemitol, isomalt, maltitol, lactitol, and combinations thereof.
85. The compound according to any one of items 81 to 84, wherein the sugar acid is selected from the group consisting of xylonic acid, gluconic acid, ascorbic acid, neuraminic acid, ketodeoxyoctonic acid, glucuronic acid, galacturonic acid, iduronic acid, mucic acid, saccharic acid, and combinations thereof.
86. The compound according to any one of items 76 to 85, wherein the organic polyol is selected from the group consisting of glycerol, sorbitol, xylitol, mannitol, maltitol, glucose, glucitol, ribulose, pentaerythritol, and trimethylolpropane.
87. The compound according to any one of items 76 to 86, wherein the compound is selected from the group consisting of

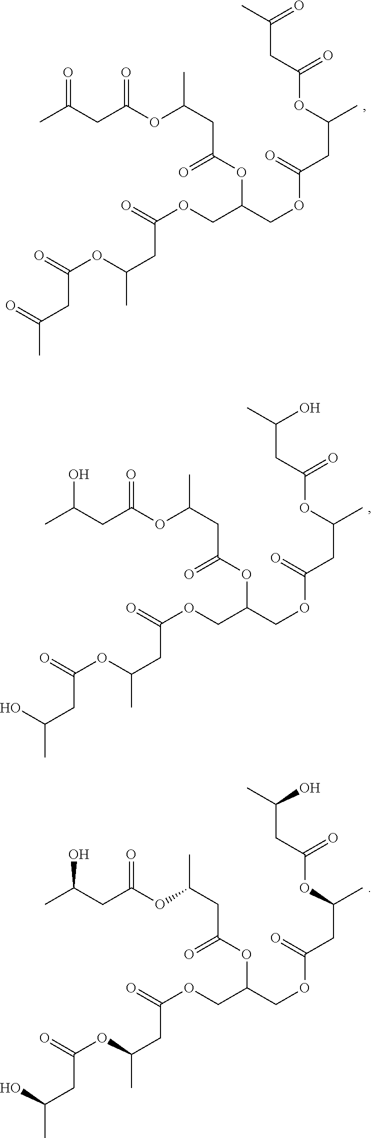 [SMILES: CC(=O)CC(=O)OC(C)CC(=O)OCC(COC(=O)CC(C)OC(=O)CC(C)=O)OC(=O)CC(C)OC(=O)CC(C)=O ; CC(O)CC(=O)OC(C)CC(=O)OCC(COC(=O)CC(C)OC(=O)CC(C)O)OC(=O)CC(C)OC(=O)CC(C)O ; C[C@H](CC(=O)OCC(COC(=O)C[C@H](C)OC(=O)C[C@@H](C)O)OC(=O)C[C@@H](C)OC(=O)C[C@@H](C)O)OC(=O)C[C@@H](C)O]

88. The compound according to any one of items 76 to 87, wherein all β-hydroxyl butyric acid ester units are either D-configured or L-configured, or all β-hydroxyl butyric acid ester units are present as a non-racemic mixture of D- and L-configurations.
89. The compound according to any one of items 76 to 88, wherein the compound contains more D-configured β-hydroxyl butyric acid ester units than L-configured β-hydroxyl butyric acid ester units, preferably wherein all β-hydroxyl butyric acid ester units are in D-configuration.
90. The compound according to any one of items 76 to 89, wherein y is from 3 to the number of hydroxyl groups of the initial polyol A, y is from 4 to the number of hydroxyl groups of the initial polyol A, or y is equal to the number of hydroxyl groups of the initial polyol A.
91. The compound or process according to any one of items 1 to 90, wherein all $-hydroxyl butyric acid ester units are in R-configuration.

It will be obvious for a person skilled in the art that these embodiments and items only depict examples of a plurality of possibilities. Hence, the embodiments shown here should not be understood to form a limitation of these features and configurations. Any possible combination and configuration of the described features can be chosen according to the scope of the invention. All embodiments and preferred embodiments described herein in connection with one particular aspect of the invention (e.g. the inventive preservative composition) shall likewise apply to all other aspects of the present inventions such as end-use formulations, uses or methods according to the present invention.

The present invention will be further illustrated by the following examples.

### EXAMPLES

### Example 1

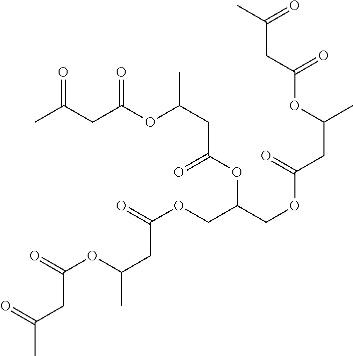 [SMILES: CC(=O)CC(=O)OC(C)CC(=O)OCC(COC(=O)CC(C)OC(=O)CC(C)=O)OC(=O)CC(C)OC(=O)CC(C)=O]

Propane-1,2,3-triyl tris(3-hydroxybutanoate) (180.0 g, 514 mmol, 1 eq.) was introduced into a stirred tank reactor. DABCO (70 mg, 0.7 mmol, 0.0013 eq.) was added and the mixture was stirred to obtain a homogenous mixture. Subsequently, diketene (129.6 g, 1.5 mol, 1 eq. per hydroxyl group) was slowly dosed to the reaction mixture while cooling the reactor jacket to maintain an internal temperature of 40-70° C. The dosing rate was adjusted in order to maintain an internal temperature of 40-70° C. After complete addition the mixture was maintained at an internal temperature of 40-70° C. for an additional 30 min. Finally the reaction mixture was cooled to room temperature and analyzed. The final product propane-1,2,3-triyl tris(3-((3-oxobutanoyl)oxy)butanoate) was obtained in quantitative yield and a purity of 64%-a/a (by HPLC at 220 nm). ¹H NMR (400 MHz, DMSO-d₆) δ ppm 1.05-1.10 (m, 9H) 1.19-1.26 (m, 9H) 2.24-2.38 (m, 6H) 2.54-2.73 (m, 6H) 3.91-4.01 (m, 3H) 4.09-4.19 (m, 2H) 4.20-4.32 (m, 2H) 4.57-4.76 (m, 3H) 5.05-5.15 (m, 3H) 5.16-5.23 (m, 1H).

### Example 2

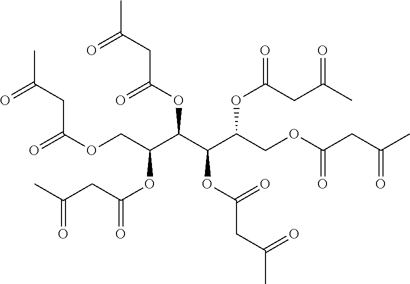 [SMILES: CC(=O)CC(=O)OC[C@H](OC(=O)CC(C)=O)[C@@H](OC(=O)CC(C)=O)[C@H](OC(=O)CC(C)=O)[C@@H](COC(=O)CC(C)=O)OC(=O)CC(C)=O]

Sorbitol (800 g, 4.39 mol, 1 eq.) was introduced into a stirred tank reactor and ethyl acetate (1.6 l, 2 relative volumes) was added. DABCO (0.64 g, 5.7 mmol, 0.0013 eq.) was added to the suspension. Subsequently, diketene (2.24 kg, 26.61 mol, 1 eq. per hydroxyl group) was slowly dosed to the reaction mixture while cooling the reactor jacket to maintain an internal temperature of 30-50° C. The dosing rate was adjusted in order to maintain an internal temperature of 30-50° C. After complete addition the mixture was maintained at an internal temperature of 50° C. for an additional 30 min before cooling to room temperature. Subsequently, Water (800 mL, 1 relative volume) and Sulfuric Acid 96%-w/w (4 g) were added and the mixture was stirred for 10 min. The aqueous phase was drained and the solvent from the organic phase was evaporated and the reaction mixture was analyzed. The final product (2R,3R,4R,5S)-hexane-1,2,3,4,5,6-hexayl hexakis(3-oxobutanoate) was obtained in quantitative yield and a purity of 72%-a/a (by HPLC at 220 nm). 1H NMR (400 MHz, DMSO-d6) δ ppm 2.11-2.28 (m, 18H) 3.45-3.82 (m, 12H) 4.10-4.35 (m, 3H) 4.35-4.50 (m, 1H) 4.98-5.15 (m, 1H) 5.26-5.35 (m, 1H) 5.35-5.44 (m, 1H) 5.44-5.55 (m, 1H).

### Example 3

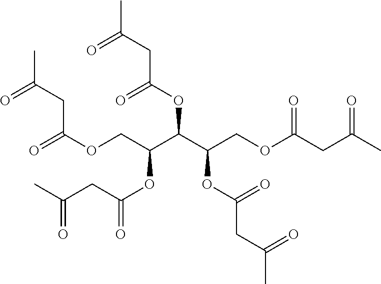 [SMILES: CC(=O)CC(=O)OC[C@H](OC(=O)CC(C)=O)[C@@H](OC(=O)CC(C)=O)[C@@H](COC(=O)CC(C)=O)OC(=O)CC(C)=O]

Xylitol (50 g, 329 mmol, 1 eq.) was introduced into a stirred tank reactor and DABCO (0.37 g, 3, 0.01 eq.) was added to the suspension. Subsequently, diketene (143.7 g, 1.7 mol, 1.04 eq. per hydroxyl group) was slowly dosed to the reaction mixture while cooling the reactor jacket to maintain an internal temperature of 50-100° C. The dosing rate was adjusted in order to maintain an internal temperature of 50-100° C. After complete addition the mixture was maintained at an internal temperature of 100° C. for an additional 30 min and the reaction mixture was cooled to room temperature and analyzed. The final product (2R,3R,4S)-pentane-1,2,3,4,5-pentayl pentakis(3-oxobutanoate) was obtained in quantitative yield. 1H NMR (400 MHz, DMSO-d6) δ ppm 2.19 (s, 15H) 3.49-3.79 (m, 10H) 3.88-4.12 (m, 2H) 4.23-4.37 (m, 2H) 5.07-5.21 (m, 1H) 5.29-5.41 (m, 1H) 5.41-5.68 (m, 1H).

### Example 4

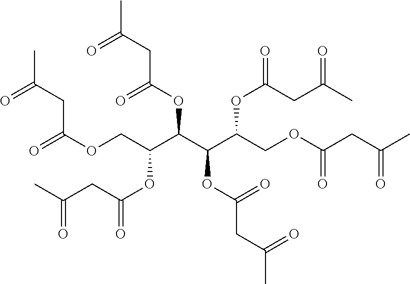 [SMILES: CC(=O)CC(=O)OC[C@@H](OC(=O)CC(C)=O)[C@@H](OC(=O)CC(C)=O)[C@H](OC(=O)CC(C)=O)[C@@H](COC(=O)CC(C)=O)OC(=O)CC(C)=O]

Mannitol (20 g, 110 mmol, 1 eq.) was introduced into a stirred tank reactor and Acetone (100 ml, 5 rel. volumes)) was added. DABCO (0.12 g, 1.1 mmol, 0.01 eq.) was added to the suspension.

Subsequently, diketene (57.2 g, 52.5 mmol, 1.03 eq. per hydroxyl group) was slowly dosed to the reaction mixture while cooling the reactor jacket to maintain reflux at 40° C. The dosing rate was adjusted in order to maintain reflux. After complete addition the mixture was maintained at reflux for an additional 30 min and the solvent was evaporated. Finally the reaction mixture was cooled to room temperature, filtered and analyzed. The final product (2R,3R,4R,5R)-hexane-1,2,3,4,5,6-hexayl hexakis(3-oxobutanoate) was isolated in a yield of 90% and a purity of 85.3%-a/a (by HPLC at 220 nm), 1H NMR (400 MHz, DMSO-d6) δ ppm 2.17-2.23 (m, 18H) 3.50-3.81 (m, 12H) 4.11-4.30 (m, 2H) 4.35-4.53 (m, 2H) 5.04-5.25 (m, 2H) 5.37-5.57 (m, 2H).

### Example 5

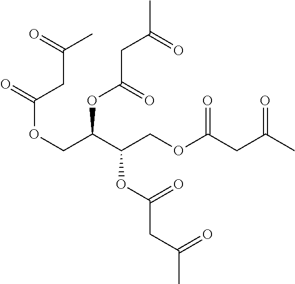 [SMILES: CC(=O)CC(=O)OC[C@H](OC(=O)CC(C)=O)[C@@H](COC(=O)CC(C)=O)OC(=O)CC(C)=O]

Erythritol (50 g, 555 mmol, 1 eq.) was introduced into a stirred tank reactor and ethyl acetate (150 ml 3 relative volumes) was added. DABCO (82 mg, 0.72 mmol, 0.0013 eq.) was added to the suspension. Subsequently, diketene (195.9 g, 2.33 mol, 1.05 eq. per hydroxyl group) was slowly dosed to the reaction mixture while cooling the reactor jacket to maintain an internal temperature of 50-65° C. The dosing rate was adjusted in order to maintain an internal temperature of 50-65° C. After complete addition the mixture was maintained at an internal temperature of 60° C. for an additional 30 min and the solvent was evaporated. Finally the reaction mixture was cooled to room temperature and analyzed. The final product (2R,3S)-butane-1,2,3,4-tetrayl tetrakis(3-oxobutanoate) was isolated in quantitative yield. LC-MS: 459.14 [M+H]⁺, 1H NMR (400 MHz, DMSO-d6) δ ppm 2.19 (br s, 12H) 3.63 (d, J=16.81 Hz, 8H) 4.24-4.31 (m, 2H) 4.31-4.56 (m, 2H) 5.17-5.38 (m, 2H).

### Example 6

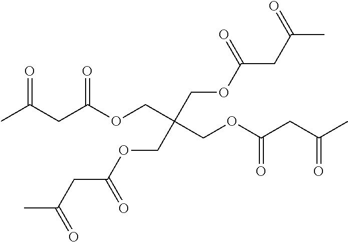 [SMILES: CC(=O)CC(=O)OCC(COC(=O)CC(C)=O)(COC(=O)CC(C)=O)COC(=O)CC(C)=O]

Pentaerythritol (40 g, 294 mmol, 1 eq.) was introduced into a stirred tank reactor and acetone (150 ml 2.5 relative volumes) was added. DABCO (333 mg, 2.9 mmol, 0.01 eq.) was added to the suspension. Subsequently, diketene (98.8 g, 1.17 mol, 1.0 eq. per hydroxyl group) was slowly dosed to the reaction mixture while cooling the reactor jacket to maintain an internal temperature of 30-40° C. The dosing rate was adjusted in order to maintain an internal temperature of 30-40° C. After complete addition the mixture was maintained at an internal temperature of 40° C. for an additional 30 min and the solvent was evaporated. Finally the reaction mixture was cooled to room temperature and analyzed. The final product 2,2-bis(((3-oxobutanoyl)oxy)methyl)propane-1,3-diyl bis(3-oxobutanoate) was isolated in quantitative yield and purity of 86.2%-a/a (by HPLC at 220 nm). 1H NMR (400 MHz, DMSO-d6) δ ppm 2.12-2.25 (m, 12H) 3.57-3.72 (m, 8H) 4.07-4.30 (m, 8H).

### Example 7

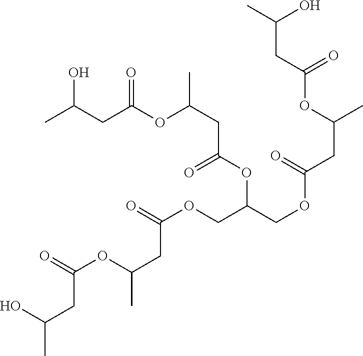 [SMILES: CC(O)CC(=O)OC(C)CC(=O)OCC(COC(=O)CC(C)OC(=O)CC(C)O)OC(=O)CC(C)OC(=O)CC(C)O]

Propane-1,2,3-triyl tris(3-hydroxybutanoate) (100 g, 175 mmol, 1 eq.) was placed in an autoclave and ethyl acetate (400 ml, 4 relative volumes) was added. Then catalyst (RuO₂, 232 mg, 1.7 mmol, 0.01 eq.) was added and the atmosphere was exchanged by pressurizing the reactor three times with nitrogen, followed by pressurizing three times with hydrogen. The hydrogen pressure was adjusted to 10-20 bar and the mixture was heated to 60° C. with 1000 rpm stirring until no further hydrogen uptake was observed (approx. 12 h). Subsequently the mixture was cooled to room temperature and the hydrogen atmosphere was exchanged with nitrogen. The reaction mixture was mixed with activated charcoal and filtered over celite. The solvent was evaporated from the filtrate and the product was analyzed. The final product propane-1,2,3-triyl tris(3-((3-hydroxybutanoyl)oxy) butanoate) was isolated in a yield of 87% and a purity of 58%-a/a (by HPLC at 220 nm). 1H NMR (400 MHz, DMSO-d6) δ ppm 1.08 (dd, J=6.27, 1.38 Hz, 9H) 1.21 (br d, J=6.27 Hz, 9H) 2.24-2.38 (m, 6H) 2.54-2.73 (m, 6H) 3.91-4.01 (m, 3H) 4.09-4.19 (m, 2H) 4.20-4.32 (m, 2H) 4.57-4.76 (m, 3H) 5.05-5.15 (m, 3H) 5.16-5.23 (m, 1H).

### Example 8

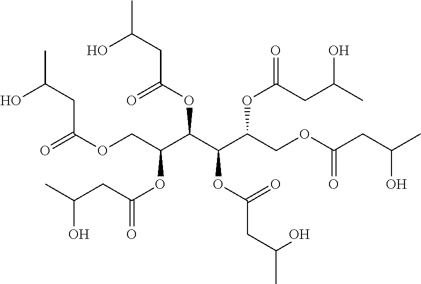 [SMILES: CC(O)CC(=O)OC[C@H](OC(=O)CC(C)O)[C@@H](OC(=O)CC(C)O)[C@H](OC(=O)CC(C)O)[C@@H](COC(=O)CC(C)O)OC(=O)CC(C)O]

(2R,3R,4R,5S)-hexane-1,2,3,4,5,6-hexayl hexakis(3-oxobutanoate) (1.2 kg, 1.75 mol, 1 eq.) was placed in an autoclave and ethyl acetate (300 ml, 0.25 relative volumes) was added. Then catalyst (RuO₂, 2.33 g, 17.5 mmol, 0.01 eq.) was added and the atmosphere was exchanged by pressurizing the reactor three times with nitrogen, followed by pressurizing three times with hydrogen. The hydrogen pressure was adjusted to 10-20 bar and the mixture was heated to 60° C. with 1000 rpm stirring until no further hydrogen uptake was observed (18 h). Subsequently the mixture was cooled to room temperature and the hydrogen atmosphere was exchanged with nitrogen. The reaction mixture was mixed with activated charcoal and filtered over celite. The solvent was evaporated from the filtrate and the product was analyzed. The final product (2R,3R,4R,5S)-hexane-1,2,3,4,5,6-hexayl hexakis(3-hydroxybutanoate) was isolated in a yield of 92% and a purity of 64%-a/a (by HPLC at 220 nm), HPLC-MS: 699.3 [M+H], 1H NMR (400 MHz, DMSO-d6) δ ppm 0.96-1.15 (m, 18H) 2.17-2.49 (m, 12H) 3.83-4.12 (m, 8H) 4.12-4.49 (m, 2H) 4.51-4.88 (m, 6H) 4.91-5.10 (m, 1H) 5.10-5.31 (m, 1H) 5.31-5.37 (m, 1H) 5.37-5.59 (m, 1H).

### Example 9

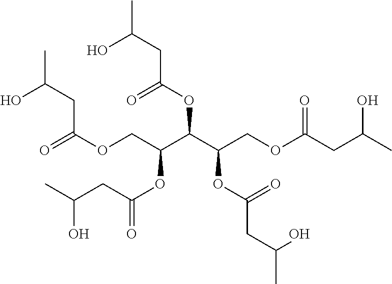 [SMILES: CC(O)CC(=O)OC[C@H](OC(=O)CC(C)O)[C@@H](OC(=O)CC(C)O)[C@@H](COC(=O)CC(C)O)OC(=O)CC(C)O]

(2R,3R,4S)-pentane-1,2,3,4,5-pentayl pentakis(3-oxobutanoate) (100 g, 175 mmol, 1 eq.) was placed in an autoclave and ethyl acetate (400 ml, 4 relative volumes) was added. Then catalyst (RuO₂, 232 mg, 1.75 mmol, 0.01 eq.) was added and the atmosphere was exchanged by pressurizing the reactor three times with nitrogen, followed by pressurizing three times with hydrogen. The hydrogen pressure was adjusted to 10-20 bar and the mixture was heated to 60° C. with 1000 rpm stirring until no further hydrogen uptake was observed (36 h). Subsequently the mixture was cooled to room temperature and the hydrogen atmosphere was exchanged with nitrogen. The reaction mixture was mixed with activated charcoal and filtered over celite. The solvent was evaporated from the filtrate and the product was analyzed. The final product (2R,3R,4S)-pentane-1,2,3,4,5-pentayl pentakis(3-hydroxybutanoate) was isolated in a yield of 87.8%. 1H NMR (400 MHz, DMSO-d6) δ ppm 0.95-1.13 (m, 15H) 2.22-2.48 (m, 10H) 3.82-4.12 (m, 7H) 4.12-4.33 (m, 2H) 4.57-4.79 (m, 5H) 5.00-5.20 (m, 1H) 5.21-5.36 (m, 1H) 5.36-5.55 (m, 1H).

### Example 10

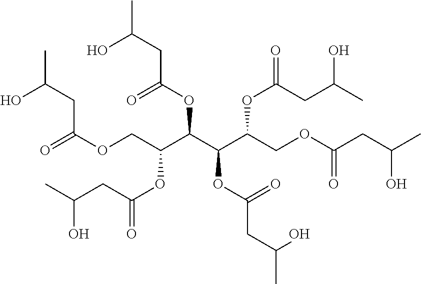 [SMILES: CC(O)CC(=O)OC[C@@H](OC(=O)CC(C)O)[C@@H](OC(=O)CC(C)O)[C@H](OC(=O)CC(C)O)[C@@H](COC(=O)CC(C)O)OC(=O)CC(C)O]

(2R,3R,4R,5R)-hexane-1,2,3,4,5,6-hexayl hexakis(3-oxobutanoate) (100 g, 146 mmol, 1 eq.) was placed in an autoclave and ethyl acetate (200 ml, 2 relative volumes) was added. Then catalyst (RuO₂, 194 mg, 1.46 mmol, 0.01 eq.) was added and the atmosphere was exchanged by pressurizing the reactor three times with nitrogen, followed by pressurizing three times with hydrogen. The hydrogen pressure was adjusted to 10-20 bar and the mixture was heated to 60° C. with 1000 rpm stirring until no further hydrogen uptake was observed (120 h). Subsequently the mixture was cooled to room temperature and the hydrogen atmosphere was exchanged with nitrogen. The reaction mixture was mixed with activated charcoal and filtered over celite. The solvent was evaporated from the filtrate and the product was analyzed. The final product (2R,3R,4R,5R)-hexane-1,2,3,4,5,6-hexayl hexakis(3-hydroxybutanoate) was isolated in a yield of 82.6%. 1H NMR (400 MHz, DMSO-d6) δ ppm 1.02-1.16 (m, 18H) 2.29-2.47 (m, 12H) 3.92-4.02 (m, 6H) 4.09-4.20 (m, 1H) 4.21-4.45 (m, 3H) 4.55-4.83 (m, 6H) 4.91-5.26 (m, 3H) 5.30-5.41 (m, 1H).

### Example 11

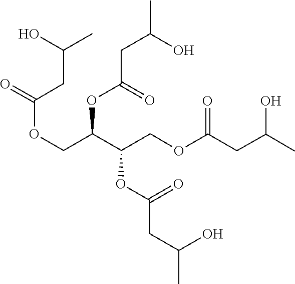 [SMILES: CC(O)CC(=O)OC[C@H](OC(=O)CC(C)O)[C@@H](COC(=O)CC(C)O)OC(=O)CC(C)O]

(2R,3S)-butane-1,2,3,4-tetrayl tetrakis(3-oxobutanoate) (200 g, 436 mmol, 1 eq.) was placed in an autoclave and ethyl acetate (300 ml, 1.5 relative volumes) was added. Then catalyst (RuO₂, 581 mg, 4.4 mmol, 0.01 eq.) was added and the atmosphere was exchanged by pressurizing the reactor three times with nitrogen, followed by pressurizing three times with hydrogen. The hydrogen pressure was adjusted to 10-20 bar and the mixture was heated to 60° C. with 1000 rpm stirring until no further hydrogen uptake was observed (18 h). Subsequently the mixture was cooled to room temperature and the hydrogen atmosphere was exchanged with nitrogen. The reaction mixture was mixed with activated charcoal and filtered over celite. The solvent was evaporated from the filtrate and the product was analyzed. The final product (2R,3S)-butane-1,2,3,4-tetrayl tetrakis(3-hydroxybutanoate) was isolated in a yield of 86.0%. LC-MS: 467.21 [M+H]⁺, 1H NMR (400 MHz, DMSO-d6) δ ppm 0.97-1.13 (m, 12H) 2.21-2.45 (m, 8H) 4.01 (s, 5H) 4.07-4.23 (m, 2H) 4.23-4.40 (m, 2H) 4.54-4.77 (m, 4H) 5.14-5.28 (m, 2H).

### Example 12

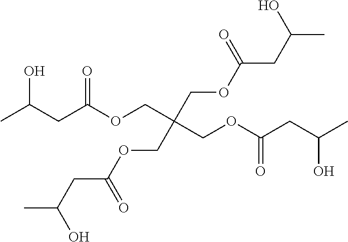 [SMILES: CC(O)CC(=O)OCC(COC(=O)CC(C)O)(COC(=O)CC(C)O)COC(=O)CC(C)O]

(2R,3S)-butane-1,2,3,4-tetrayl tetrakis(3-oxobutanoate) (50 g, 106 mmol, 1 eq.) was placed in an autoclave and ethyl acetate (300 ml, 1.5 relative volumes) was added. Then catalyst (RuO₂, 146 mg, 1.1 mmol, 0.01 eq.) was added and the atmosphere was exchanged by pressurizing the reactor three times with nitrogen, followed by pressurizing three times with hydrogen. The hydrogen pressure was adjusted to 10-20 bar and the mixture was heated to 60° C. with 1000 rpm stirring until no further hydrogen uptake was observed (36 h). Subsequently the mixture was cooled to room temperature and the hydrogen atmosphere was exchanged with nitrogen. The reaction mixture was mixed with activated charcoal and filtered over celite. The solvent was evaporated from the filtrate and the product was analyzed. The final product 2,2-bis(((3-hydroxybutanoyl)oxy)methyl)propane-1,3-diyl bis(3-hydroxybutanoate) was isolated in a yield of 81.4%. 1H NMR (400 MHz, DMSO-d6) δ ppm 0.85 (d, J=6.27 Hz, 12H) 2.07-2.17 (m, 8H) 3.66-3.79 (m, 4H) 3.79-3.95 (m, 8H) 4.32-4.63 (m, 4H).

### Example 13

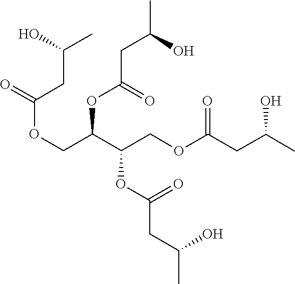 [SMILES: C[C@@H](O)CC(=O)OC[C@H](OC(=O)C[C@@H](C)O)[C@@H](COC(=O)C[C@@H](C)O)OC(=O)C[C@@H](C)O]

(2R,3S)-butane-1,2,3,4-tetrayl tetrakis(3-oxobutanoate) (25 g, 54.5 mmol, 1 eq.) was placed in an autoclave and methanol (77 ml, 35 eq.) was added. Then catalyst ([RuCl₂((R)-BINAP)]₂NEt₃, 218 mg, 0.11 mmol, 0.002 eq.) and H₂SO₄ (2N, 97 mg, 0.002 eq.) was added and the atmosphere was exchanged by pressurizing the reactor three times with nitrogen, followed by pressurizing three times with hydrogen. The hydrogen pressure was adjusted to 30 bar and the mixture was heated to 60° C. with 600 rpm stirring until no further hydrogen uptake was observed (5d). Subsequently the mixture was cooled to room temperature and the hydrogen atmosphere was exchanged with nitrogen. The reaction mixture was mixed with activated charcoal and filtered over celite an silica. The solvent was evaporated from the filtrate and the product was analyzed. The final product (2R,3S)-butane-1,2,3,4-tetrayl (3R,3′R,3″R,3′″)-tetrakis(3-hydroxybutanoate) was isolated as a brown liquid in quantitative yield (ee=95.4%). 1H NMR (400 MHz, CDCl₃) δ ppm 1.28 (m, 12H), 2.50 (m, 8H), 4.39 (m, 8H), 5.25 (m, 2H).

### Example 14

Meso-Erythritol (6 g, 0.05 mol, 1 eq.) was introduced into a stirred tank reactor and ethyl acetate (10.8 g, 2.5 eq.) was added. DABCO (7.2 mg, 0.0001 mol, 0.0013 eq.) was added to the suspension. Subsequently, diketene (4.1 g, 0.05 mol, 1 eq.) was slowly dosed to the reaction mixture over 8 h while cooling the reactor jacket to maintain an internal temperature of 40° C. The dosing rate was adjusted in order to maintain an internal temperature of 40° C. After complete addition the mixture was maintained at an internal temperature of 40° C. overnight. The solvent was removed under reduced pressure to obtain a mixture of isomers of meso-erythritol monoacetoacetate (7.2 g, 72%) as a white-yellow solid. 1H NMR (400 MHz, DMSO-d₆) δ ppm 2.18 (m, 3H), 3.39 (s, 5H), 4.35 (m, 2H), 4.48 (s, 2H).

### Example 15

Meso-Erythritol (6 g, 0.05 mol, 1 eq.) was introduced into a stirred tank reactor and ethyl acetate (10.8 g, 2.5 eq.) was added. DABCO (7.2 mg, 0.0001 mol, 0.0013 eq.) was added to the suspension.

Subsequently, diketene (8.3 g, 0.1 mol, 2 eq.) was slowly dosed to the reaction mixture over 8 h while cooling the reactor jacket to maintain an internal temperature of 40° C. The dosing rate was adjusted in order to maintain an internal temperature of 40° C. After complete addition the mixture was maintained at an internal temperature of 40° C. overnight. The solvent was removed under reduced pressure to obtain a mixture of isomers of meso-erythritol diacetoacetate (13.4 g, 94%) as an orange solid. 1H NMR (400 MHz, DMSO-d₆) δ ppm 2.18 (m, 6H), 3.38 (m, 6H), 3.54 (m, 2H), 4.24 (m, 1H), 4.43 (m, 1H), 4.48 (s, 1H), 5.52 (m, 1H).

### Example 16

Meso-Erythritol (6 g, 0.05 mol, 1 eq.) was introduced into a stirred tank reactor and ethyl acetate (10.8 g, 2.5 eq.) was added. DABCO (7.2 mg, 0.0001 mol, 0.0013 eq.) was added to the suspension. Subsequently, diketene (12.4 g, 0.15 mol, 3 eq.) was slowly dosed to the reaction mixture over 8 h while cooling the reactor jacket to maintain an internal temperature of 40° C. The dosing rate was adjusted in order to maintain an internal temperature of 40° C. After complete addition the mixture was maintained at an internal temperature of 40° C. overnight. The solvent was removed under reduced pressure to obtain a mixture of isomers of meso-erythritol triacetoacetate (18.1 g, 99%) as a yellow suspension. 1H NMR (400 MHz, DMSO-d₆) δ ppm 2.18 (m, 9H), 3.37 (m, 7H), 3.61 (m, 3H), 3.65 (m, 3H), 4.23 (m, 1H), 4.35 (m, 2H), 5.25 (m, 1H).

### Example 17

A mixture of isomers of meso-erythritol monoacetoacetate (6.9 g, 0.03 mol, 1 eq., Example 14) was placed in an autoclave with ethyl acetate (141 g, 41 eq.). RuO₂ (0.08 g, 0.6 mmol, 0.02 eq.) was added and the atmosphere was exchanged by pressurizing the reactor three times with nitrogen, followed by pressurizing three times with hydrogen. The hydrogen pressure was adjusted to 20 bar and the mixture was heated to 60° C. with 1000 rpm stirring the possible hydrogen uptake was observed (6d). Subsequently the mixture was cooled to room temperature and the hydrogen atmosphere was exchanged with nitrogen. The reaction mixture was mixed with activated charcoal and filtered over celite. The solvent was evaporated from the filtrate and the product was analyzed. The obtained mixture of isomers of meso-erythritol mono(3-hydroxybutanoate) was isolated as a yellow oil (3.42 g, 49%). 1H NMR (400 MHz, DMSO-d₆) δ ppm 1.09 (m, 3H), 2.36 (m, 2H), 3.36 (m, 3H), 3.94 (m, 3H), 4.30 (m, 3H).

### Example 18

A mixture of isomers of meso-erythritol diacetoacetate (11.9 g, 0.04 mol, 1 eq., Example 15) was placed in an autoclave with ethyl acetate (141 g, 41 eq.). Ru/C (5 wt %, 4 g, 2.0 mmol, 0.05 eq.) was added and the atmosphere was exchanged by pressurizing the reactor three times with nitrogen, followed by pressurizing three times with hydrogen. The hydrogen pressure was adjusted to 10 bar and the mixture was heated to 40° C. with 1000 rpm stirring the possible hydrogen uptake was observed (1d). Subsequently the mixture was cooled to room temperature and the hydrogen atmosphere was exchanged with nitrogen. The reaction mixture was mixed with activated charcoal and filtered over celite. The solvent was evaporated from the filtrate and the product was analyzed. The obtained mixture of isomers of meso-erythritol di(3-hydroxybutanoate) was isolated as a yellow oil (7.51 g, 64%). 1H NMR (400 MHz, DMSO-d₆) δ ppm 1.10 (m, 6H), 2.36 (m, 4H), 4.01 (m, 3H), 4.17 (m, 1H), 4.34 (m, 1H), 4.74 (m, 2H).

### Example 19

A mixture of isomers of meso-erythritol triacetoacetate (16.5 g, 0.04 mol, 1 eq., Example 16) was placed in an autoclave with ethyl acetate (140 g, 36 eq.). Ru/C (5 wt %, 5.5 g, 2.7 mmol, 0.06 eq.) was added and the atmosphere was exchanged by pressurizing the reactor three times with nitrogen, followed by pressurizing three times with hydrogen. The hydrogen pressure was adjusted to 10 bar and the mixture was heated to 40° C. with 1000 rpm stirring the possible hydrogen uptake was observed (1d). Subsequently the mixture was cooled to room temperature and the hydrogen atmosphere was exchanged with nitrogen. The reaction mixture was mixed with activated charcoal and filtered over celite. The solvent was evaporated from the filtrate and the product was analyzed. The obtained mixture of isomers of meso-erythritol tri(3-hydroxybutanoate) was isolated as a yellow oil (16.6 g, 99%). 1H NMR (400 MHz, DMSO-d₆) δ ppm 1.09 (m, 9H), 2.37 (m, 6H), 3.99 (m, 3H) 4.15 (m, 1H), 4.31 (m, 1H), 4.74 (m, 3H).

1.-44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. A process for the preparation of a compound of formula 1 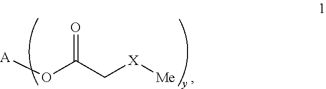
A derived from an organic polyol with at least 4 hydroxyl groups,
X being —C(H)(OH)— or —C(H)(OR¹)—,
R¹ being selected from linear or branched C₁₋₁₂ alkyl, C₃₋₈ cycloalkyl, linear or branched C₂₋₁₂ hydroxyalkyl, linear or branched C₁₋₁₂ carboxyalkly, linear or branched and saturated or unsaturated C₁₋₂₄ alkanoyl, phenyl, and carboxyphenyl, and
y being from 1 to the number of hydroxyl groups of the initial organic polyol A,
the process comprising:
reacting an organic polyol of formula 2 with diketene 3 resulting in the formation of a compound according to formula 4; 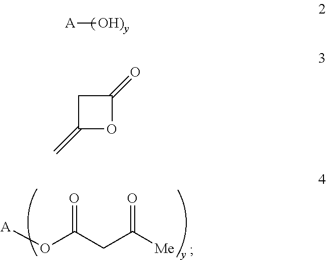
and
reacting the compound of formula 4 with hydrogen in the presence of a catalyst resulting in the formation of a compound according to formula 5,

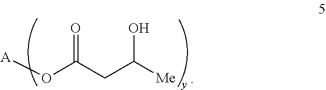

56. The process according to claim 55, further comprising reacting the compound of formula 5 with a compound LG-R¹, wherein LG is a leaving group, resulting in the formation of a compound according to formula 6

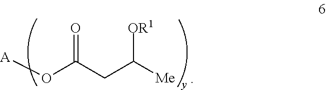

57. The process according to claim 55, wherein the organic polyol is selected from a linear or branched C₂₋₁₂ alkyl substituted with at least 4 hydroxyl groups or a C₃₋₈ cycloalkyl substituted with at least 4 hydroxyl groups.

58. The process according to claim 57, wherein one or both of:
the linear or branched C₂₋₁₂ alkyl substituted with at least 4 hydroxyl groups is selected from the group consisting of pentaerythritol, butanetetrol, pentanetetrol, hexanetetrol, and hexanepentol; and
the C₃₋₈ cycloalkyl substituted with at least 4 hydroxyl groups is selected from the group consisting of cyclopentanetetrol, cyclopentanepentol, cyclohexanetetrol, cyclohexanepentol, and cyclohexanehexol.

59. The process according to claim 55, wherein the organic polyol is selected from the group consisting of monosaccharides, sugar alcohols, and sugar acids.

60. The process according to claim 59, wherein:
the monosaccharide is selected from pentoses, hexoses, and heptoses,
the monosaccharide is selected from aldopentoses, ketopentoses, aldohexosen, ketohexoses, aldoheptoses, and ketoheptoses, or
the monosaccharide is selected from the group consisting of ribose, arabinose, xylose, lyxose, ketopentose, ribulose, xylulose, allose, altrose, glucose, mannose, gulose, idose, galactose, talose, n-acetyl-d-glucosamin, glucosamin, N-acetyl-D-galactosamin, fucose, rhamnose, chinovose, fructose, 2-desoxy-D-glucose, fluordesoxyglucose, 6-desoxyfructose, 1,6-dichlorfructose, 3,6-anhydrogalactose, 1-O-methylgalactose, 1-O-methyl-D-glucose, 1-O-methyl-D-fructose, 3-O-methyl-D-fructose, 6-O-methyl-D-galactose, sedoheptulose, mannoheptulose, L-glycero-D-manno-heptose, and combinations thereof.

61. The process according to claim 59, wherein one or both of:
the sugar alcohol is selected from the group consisting of erythritol, threitol, arabitol, xylitol, ribitol, mannitol, sorbitol, galactitol, fucitol, iditol, inositol, volemitol, isomalt, maltitol, lactitol, and combinations thereof, and
the sugar acid is selected from the group consisting of xylonic acid, gluconic acid, ascorbic acid, neuraminic acid, ketodeoxyoctonic acid, glucuronic acid, galacturonic acid, iduronic acid, mucic acid, saccharic acid, and combinations thereof.

62. The process according to claim 55, wherein the organic polyol is selected from the group consisting of sorbitol, xylitol, mannitol, erythritol, maltitol, glucose, glucitol, ribulose, and pentaerythritol.

63. The process according to claim 55, wherein y is from 3 to the number of hydroxyl groups of the initial polyol A, y is from 4 to the number of hydroxyl groups of the initial polyol A, or y is equal to the number of hydroxyl groups of the initial polyol A.

64. The process according to claim 55, wherein one of more of:
R¹ is a fatty acid residue selected from an omega fatty acid, a medium-chain fatty acid, or a combination thereof;
the omega fatty acid is an omega-3 fatty acid, an omega-6 fatty acid, an omega-3,6 fatty acid, or a combination thereof; and
the medium-chain fatty acid is selected from the group consisting of caproic acid, caprylic acid, capric acid, lauric acid and combinations thereof.

65. The process according to claim 55, wherein the compound is selected from the group consisting of

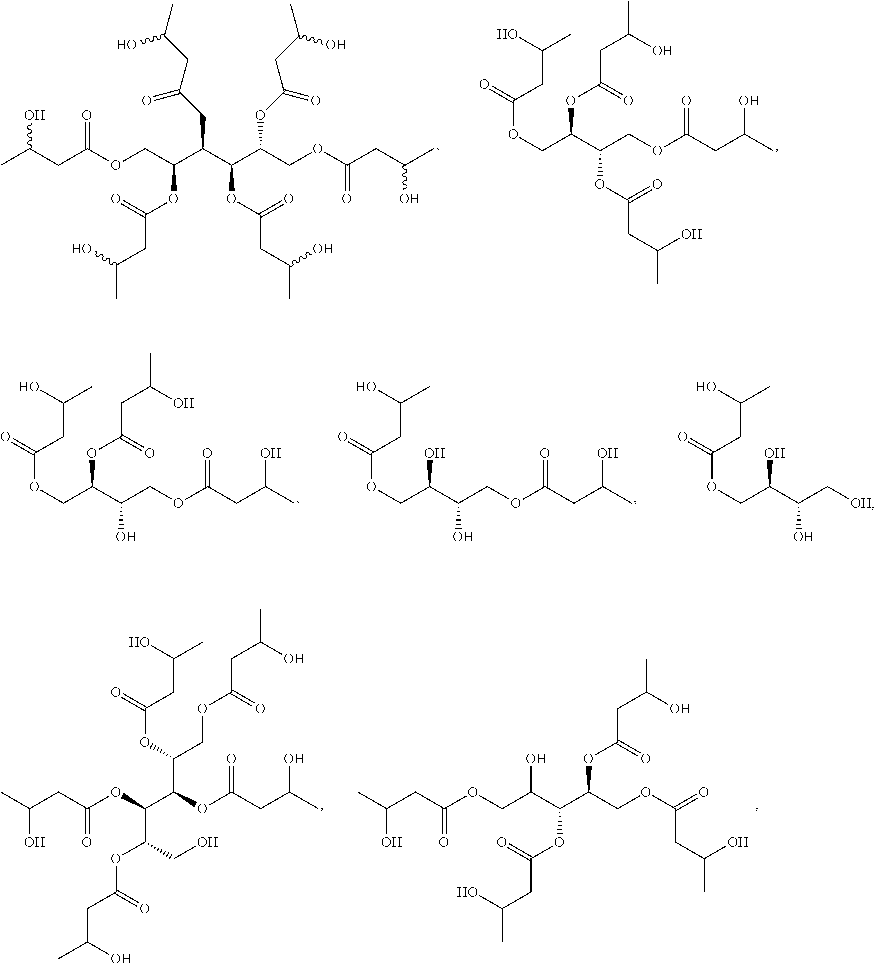

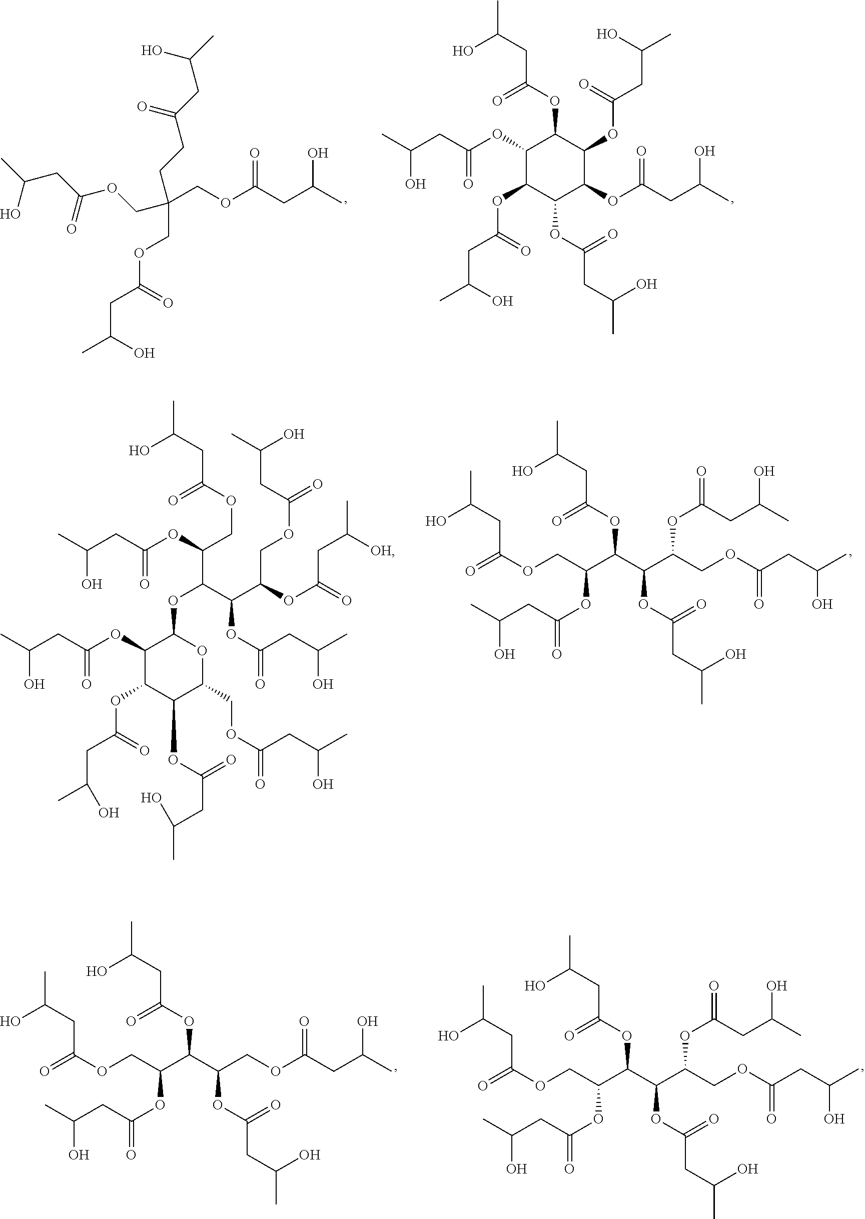

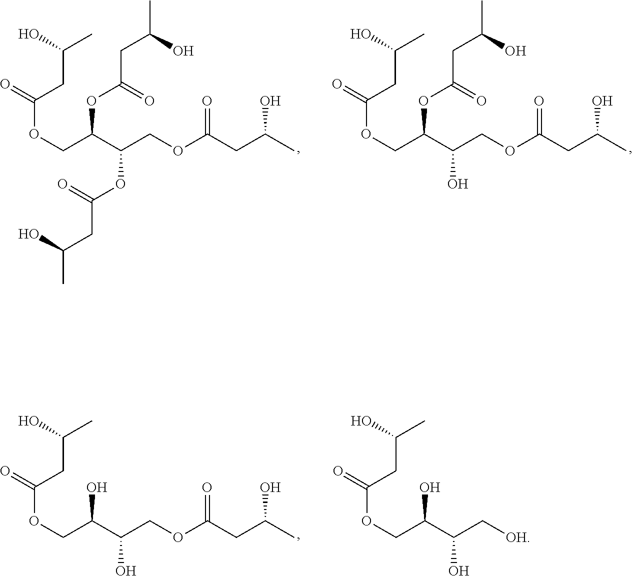 [SMILES: C[C@@H](O)CC(=O)OC[C@H](OC(=O)C[C@@H](C)O)[C@@H](COC(=O)C[C@@H](C)O)OC(=O)C[C@@H](C)O ; C[C@@H](O)CC(=O)OC[C@H](O)[C@@H](COC(=O)C[C@@H](C)O)OC(=O)C[C@@H](C)O ; C[C@@H](O)CC(=O)OC[C@@H](O)[C@@H](O)COC(=O)C[C@@H](C)O ; C[C@@H](O)CC(=O)OC[C@@H](O)[C@@H](O)CO]

66. (canceled)

67. (canceled)

68. (canceled)

69. (canceled)

70. (canceled)

71. (canceled)

72. (canceled)

73. (canceled)

74. (canceled)

75. A process for the preparation of a compound of formula 9 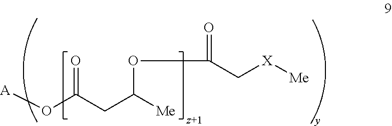
z being 0 or more,
A derived from an organic polyol with at least 3 hydroxyl groups,
X being —C(O)—, —C(H)(OH)—, or —C(H)(OR¹)—,
R¹ being selected from linear or branched C₁₋₁₂ alkyl, C₃₋₈ cycloalkyl, linear or branched C₂₋₁₂ hydroxyalkyl, linear or branched C₁₋₁₂ carboxyalkly, linear or branched and saturated or unsaturated C₁₋₂₄ alkanoyl, phenyl, and carboxyphenyl, and
y being from 1 to the number of hydroxyl groups of the initial organic polyol A,
the process comprising:
reacting a compound of formula 10 with diketene 3 resulting in the formation of a compound according to formula 11

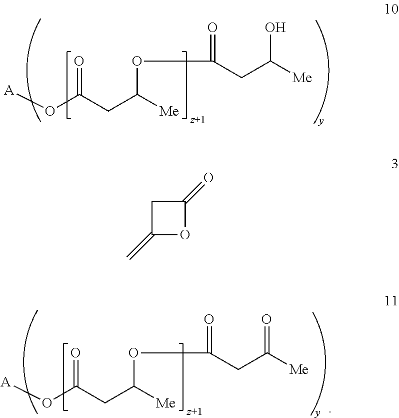

76. The process according to claim 75, further comprising reacting the compound of formula 11 with hydrogen in the presence of a catalyst resulting in the formation of a compound according to formula 12

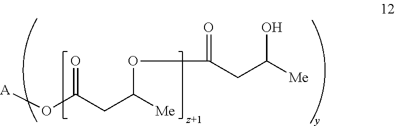

77. The process according to claim 76, further comprising reacting the compound of formula 12 with a compound LG-R¹, wherein LG is a leaving group, resulting in the formation of a compound according to formula 13

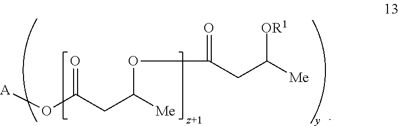

78. The process according to claim 75, wherein z is from 0-100 such as from 0-95, 0-90, 0-85, 0-80, 0-75, 0-70, 0-65, 0-60, 0-55, 0-50, 0-45, 0-40, 0-35, 0-30, 0-25, or 0-20, preferably z is from 0-20, such as 0-19, such as 0-18, such as 0-17, such as 0-16, such as 0-15, such as 0-14, such as 0-13, such as 0-12, such as 0-11, such as 0-10, such as 0-9, such as 0-8, such as 0-7, such as 0-6, such as 0-5, such as 0-4, such as 0-3, such as 0-2, more preferably wherein z is 0 or 1.

79. The process according to claim 75, wherein the organic polyol is selected from a linear or branched C₂₋₁₂ alkyl substituted with at least 3 hydroxyl groups or a C₃₋₈ cycloalkyl substituted with at least 3 hydroxyl groups.

80. The process according to claim 79, wherein one or both of:
the linear or branched C₂₋₁₂ alkyl substituted with at least 3 hydroxyl groups is selected from the group consisting of glycerol, trimethylolpropane, butanetriol, 2-methyl-propanetriol, pentanetriol, 3-methyl-pentanetriol, hexanetriol, pentaerythritol, butanetetrol, pentanetetrol, hexanetetrol, hexanepentol, and combinations thereof; and
the C₃₋₈ cycloalkyl substituted with at least 3 hydroxyl groups is selected from the group consisting of cyclopentanetriol, cyclohexanetriol, cyclopentanetetrol, cyclohexanetetrol, and combinations thereof.

81. The process according to claim 75, wherein the organic polyol is selected from the group consisting of monosaccharides, sugar alcohols, and sugar acids.

82. The process according to claim 81, wherein:
the monosaccharide is selected from tetroses, pentoses, hexoses, and heptoses;
the monosaccharide is selected from aldotetroses, ketotetroses, aldopentoses, ketopentoses, aldohexosen, ketohexoses, aldoheptoses and ketoheptoses; or
the monosaccharide is selected from the group consisting of erythrose, threose, erythrulose, ribose, arabinose, xylose, lyxose, desoxyribose, ketopentose, ribulose, xylulose, allose, altrose, glucose, mannose, gulose, idose, galactose, talose, n-acetyl-d-glucosamin, glucosamin, N-acetyl-D-galactosamin, fucose, rhamnose, chinovose, fructose, 2-desoxy-D-glucose, fluordesoxyglucose, 6-desoxyfructose, 1,6-dichlorfructose, 3,6-anhydrogalactose, 1-O-methylgalactose, 1-O-methyl-D-glucose, 1-O-methyl-D-fructose, 3-O-methyl-D-fructose, 6-O-methyl-D-galactose, sedoheptulose, mannoheptulose, L-glycero-D-manno-heptose, and combinations thereof.

83. The process according to claim 81, wherein one or both of:
the sugar alcohol is selected from the group consisting of erythritol, threitol, arabitol, xylitol, ribitol, mannitol, sorbitol, galactitol, fucitol, iditol, inositol, volemitol, isomalt, maltitol, lactitol, and combinations thereof, and
the sugar acid is selected from the group consisting of xylonic acid, gluconic acid, ascorbic acid, neuraminic acid, ketodeoxyoctonic acid, glucuronic acid, galacturonic acid, iduronic acid, mucic acid, saccharic acid, and combinations thereof.

84. The process according to claim 75, wherein the organic polyol is selected from the group consisting of glycerol, sorbitol, xylitol, mannitol, erythritol, maltitol, glucose, glucitol, ribulose, pentaerythritol, and trimethylolpropane.

85. The process according to claim 75, wherein the organic polyol is erythritol.

86. The process according to claim 75, wherein the compound is selected from the group consisting of

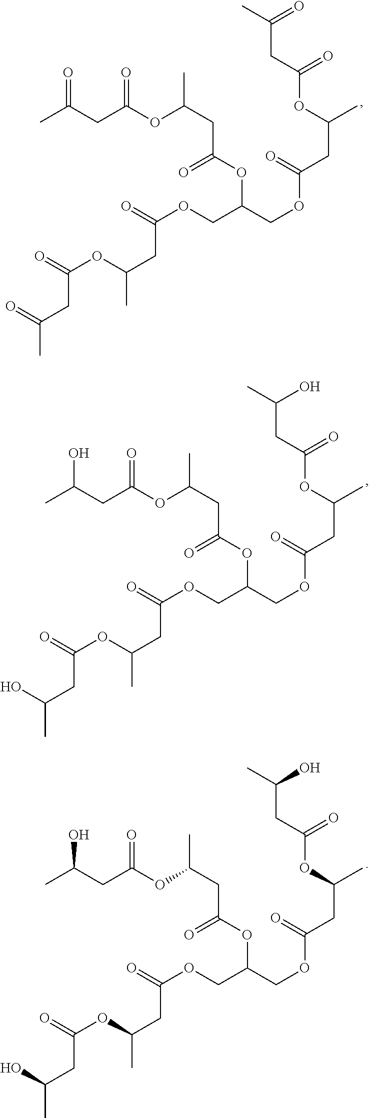 [SMILES: CC(=O)CC(=O)OC(C)CC(=O)OCC(COC(=O)CC(C)OC(=O)CC(C)=O)OC(=O)CC(C)OC(=O)CC(C)=O ; CC(O)CC(=O)OC(C)CC(=O)OCC(COC(=O)CC(C)OC(=O)CC(C)O)OC(=O)CC(C)OC(=O)CC(C)O ; C[C@H](CC(=O)OCC(COC(=O)C[C@H](C)OC(=O)C[C@@H](C)O)OC(=O)C[C@@H](C)OC(=O)C[C@@H](C)O)OC(=O)C[C@@H](C)O]

87. The process according to claim 75, wherein y is from 3 to the number of hydroxyl groups of the initial polyol A, y is from 4 to the number of hydroxyl groups of the initial polyol A, or y is equal to the number of hydroxyl groups of the initial polyol A.

88. The process according to claim 75, wherein:
R¹ is a fatty acid residue selected from an omega fatty acid, a medium-chain fatty acid, or a combination thereof;
the omega fatty acid is an omega-3 fatty acid, an omega-6 fatty acid, an omega-3,6 fatty acid, or a combination thereof; or
the medium-chain fatty acid is selected from the group consisting of caproic acid, caprylic acid, capric acid, lauric acid and combinations thereof.

89. (canceled)

90. The process according to claim 55, wherein all β-hydroxyl butyric acid ester units are in R-configuration.

91. (canceled)

92. The process according to claim 75, wherein all β-hydroxyl butyric acid ester units are in R-configuration.
